(19) United States
(12) Patent Application Publication
(10) Pub. No.: US 2023/0142913 A1
(43) Pub. Date: May 11, 2023

(54) TRICYCLIC HETEROARENES, PHARMACEUTICAL COMPOSITIONS CONTAINING THE SAME, AND METHODS OF USING THE SAME

(71) Applicant: Repare Therapeutics Inc., St-Laurent (CA)

(72) Inventors: Janek SZYCHOWSKI, Montreal (CA); Evelyne DIETRICH, Laval (CA); Frédéric VALLÉE, Montreal (CA)

(21) Appl. No.: 17/961,103

(22) Filed: Oct. 6, 2022

Related U.S. Application Data
(60) Provisional application No. 63/253,004, filed on Oct. 6, 2021.

(51) Int. Cl. C07D 471/14; C07D 471/04; C07D 221/16; C07D 487/04; C07D 487/14

(52) U.S. Cl. CPC C07D 471/14; C07D 471/04; C07D 221/16; C07D 487/04; C07D 487/14

(57) ABSTRACT

Disclosed are compounds and pharmaceutically acceptable salts thereof that may be used in the treatment of subjects in need thereof. The compounds disclosed herein may be inhibitors of tyrosine and threonine-specific cdc2-inhibitory kinase (Myt1). Also disclosed are pharmaceutical compositions containing the compounds or pharmaceutically acceptable salts thereof and methods of their preparation and use.

### FIELD OF THE INVENTION

The invention relates to compounds and pharmaceutical compositions, their preparation and their use in the treatment of a disease or condition, e.g., cancer, and, in particular, those diseases or conditions (e.g., cancers that harbor CCNE1 amplification/overexpression or FBXW7-mutated cancers) which depend on the activity of membrane-associated tyrosine and threonine-specific cdc2-inhibitory kinase (Myt1).

### BACKGROUND

DNA is continuously subjected to both endogenous insults (e.g., stalled replication forks, reactive oxygen species) and exogenous insults (UV, ionizing radiation, chemical) that can lead to DNA damage. As a result, cells have established sophisticated mechanisms to counteract these deleterious events that would otherwise compromise genomic integrity and lead to genomic instability diseases such as cancer. These mechanisms are collectively referred to as the DNA damage response (DDR). One component of the overall DDR is the activation of various checkpoint pathways that modulate specific DNA-repair mechanisms throughout the various phases of the cell cycle, which includes the G1, S, G2 and Mitosis checkpoints. A majority of cancer cells have lost their G1 checkpoint owing to p53 mutations and as such, rely on the G2 checkpoint to make the necessary DNA damage corrections prior to committing to enter mitosis and divide into 2 daughter cells.

There is a need for new anti-cancer therapeutic approaches, e.g., those utilizing small-molecules, especially therapies allowing for targeted cancer treatment.

### SUMMARY OF THE INVENTION

In an aspect, the invention provides a compound of formula (IA):

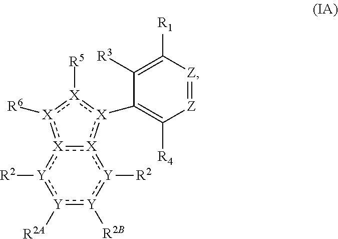

or a pharmaceutically acceptable salt thereof,
wherein

each [symbol] is a single or double bond;

one, two, or three X groups are N, and the remaining X groups are C;

each Y is independently N or C;

each Z is independently N or CH;

R¹ is OH, and R³ is hydrogen, optionally substituted C₁₋₆ alkyl, halogen, or optionally substituted C₃₋₈ cycloalkyl; or R¹ and R³ combine to form —CR⁹═N—NH—;

each R² is independently absent, hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, cyano, —N(R⁷)₂, —OR⁷, —C(O)N(R⁸)₂, —SO₂N(R⁸)₂, —SO₂R^{7A}, or -Q-R^{7B}; and R^{2A} and R^{2B}, together with the atoms to which they are attached, combine to form ring A; or R² and R^{2A}, together with the atoms to which they are attached, combine to form ring A, and R^{2B} is absent or hydrogen;

R⁴ is hydrogen, optionally substituted C₁₋₆ alkyl, halogen, or optionally substituted C₃₋₈ cycloalkyl;

R⁵ is hydrogen, halogen, or —N(R⁷)₂;

R⁶ is —C(O)NH(R⁸), —C(O)R^{7A}, or —SO₂R^{7A};

each R⁷ is independently hydrogen, optionally substituted C₁₋₆ alkyl, optionally substituted C₆₋₁₀ aryl C₁₋₆ alkyl, optionally substituted C₃₋₈ cycloalkyl, optionally substituted C₆₋₁₀ aryl, optionally substituted C₂₋₉ heterocyclyl, optionally substituted C₁₋₉ heteroaryl, optionally substituted C₁₋₉ heteroaryl C₁₋₆ alkyl, or —SO₂R^{7A}; or two R⁷ groups, together with the atom to which both are attached, combine to form an optionally substituted C₂₋₉ heterocyclyl;

each R^{7A} is independently optionally substituted C₁₋₆ alkyl, optionally substituted C₃₋₈ cycloalkyl, or optionally substituted C₆₋₁₀ aryl;

each R^{7B} is independently hydroxyl, optionally substituted C₁₋₆ alkyl, optionally substituted C₆₋₁₀ aryl, optionally substituted C₂₋₉ heterocyclyl, optionally substituted C₁₋₉ heteroaryl, —N(R⁷)₂, —C(O)N(R⁸)₂, —SO₂N(R⁸)₂, —SO₂R^{7A}, or optionally substituted alkoxy;

each R⁸ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or two R⁸, together with the atom to which they are attached, combine to form an optionally substituted C₂₋₉ heterocyclyl;

R⁹ is hydrogen or halogen;

ring A is a 5- or 6-membered carbocyclic ring or a 5- or 6-membered heterocyclic ring, wherein A is optionally substituted with 1, 2, 3, or 4 non-hydrogen R² groups; and

Q is optionally substituted C₁₋₆ alkylene, optionally substituted C₂₋₆ alkenylene, optionally substituted C₂₋₆ alkynylene, optionally substituted C₃₋₈ cycloalkylene, optionally substituted C₃₋₈ cycloalkenylene optionally substituted C₆₋₁₀ arylene, optionally substituted C₂₋₉ heterocyclylene, or optionally substituted C₁₋₉ heteroarylene;

wherein each R² is absent, if attached to Y that is N.

In some embodiments, the compound is a compound of formula (I):

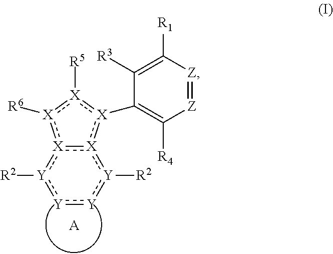

or a pharmaceutically acceptable salt thereof, where

each [symbol] is a single or double bond;

A is a 5- or 6-membered carbocyclic ring or a 5- or 6-membered heterocyclic ring, where A is optionally substituted with 1, 2, 3, or 4 non-hydrogen R² groups;

one, two, or three X groups are N, and the remaining X groups are C;

each Y is independently N or C;

each Z is independently N or CH;

R¹ is OH, and R³ is hydrogen, optionally substituted C₁₋₆ alkyl, halogen, or optionally substituted C₃₋₈ cycloalkyl; or R¹ and R³ combine to form —CR⁹═N—NH—;

each R² is independently absent, hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, cyano, —N(R⁷)₂, —OR⁷, —C(O)N(R⁸)₂, —SO₂N(R⁸)₂, —SO₂R^{7A}, or -Q-R^{7B};

R⁴ is hydrogen, optionally substituted C₁₋₆ alkyl, halogen, or optionally substituted C₃₋₈ cycloalkyl;

R⁵ is hydrogen, halogen, or —N(R⁷)₂;

R⁶ is —C(O)NH(R⁸), —C(O)R^{7A}, or —SO₂R^{7A};

each R⁷ is independently hydrogen, optionally substituted C₁₋₆ alkyl, optionally substituted C₆₋₁₀ aryl C₁₋₆ alkyl, optionally substituted C₃₋₈ cycloalkyl, optionally substituted C₆₋₁₀ aryl, optionally substituted C₂₋₉ heterocyclyl, optionally substituted C₁₋₉ heteroaryl, optionally substituted C₁₋₉ heteroaryl C₁₋₆ alkyl, or —SO₂R^{7A}; or two R⁷ groups, together with the atom to which both are attached, combine to form an optionally substituted C₂₋₉ heterocyclyl;

each R^{7A} is independently optionally substituted C₁₋₆ alkyl, optionally substituted C₃₋₈ cycloalkyl, or optionally substituted C₆₋₁₀ aryl;

each R^{7B} is independently hydroxyl, optionally substituted C₁₋₆ alkyl, optionally substituted C₆₋₁₀ aryl, optionally substituted C₂₋₉ heterocyclyl, optionally substituted C₁₋₉ heteroaryl, —N(R⁷)₂, —C(O)N(R⁸)₂, —SO₂N(R⁸)₂, —SO₂R^{7A}, or optionally substituted alkoxy;

each R⁸ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or two R⁸, together with the atom to which they are attached, combine to form an optionally substituted C₂₋₉ heterocyclyl;

R⁹ is hydrogen or halogen; and

Q is optionally substituted C₁₋₆ alkylene, optionally substituted C₂₋₆ alkenylene, optionally substituted C₂₋₆ alkynylene, optionally substituted C₃₋₈ cycloalkylene, optionally substituted C₃₋₈ cycloalkenylene optionally substituted C₆₋₁₀ arylene, optionally substituted C₂₋₉ heterocyclylene, or optionally substituted C₁₋₉ heteroarylene;

where each R² is absent, if attached to Y that is N.

In some embodiments, one X group is N, and the remaining X groups are C. In some embodiments, all Y groups are C. In some embodiments, one Y group is N, and the remaining Y groups are C. In some embodiments, two Y group are N, and the remaining Y groups are C.

In some embodiments, the compound is of formula (II):

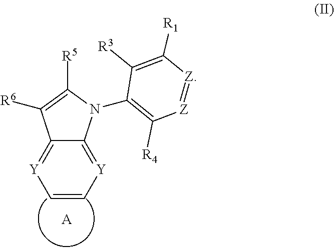

In some embodiments, the compound is enriched for the atropisomer of formula (II-i):

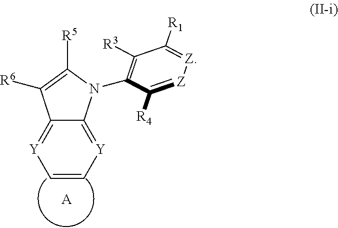

In some embodiments, the compound is of formula (IIA):

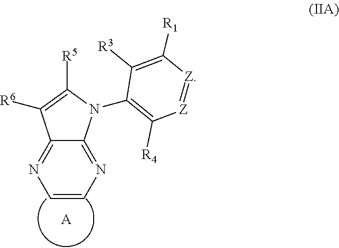

In some embodiments, the compound is enriched for the atropisomer of formula (IIA-i):

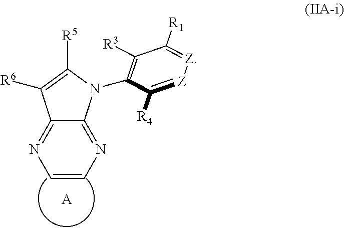

In some embodiments, the compound is of formula (IIA-ii):

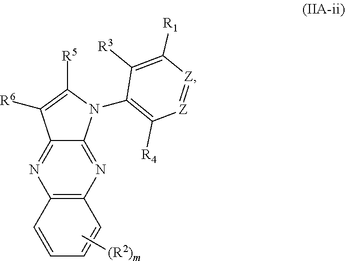

where m is 0, 1, 2, 3, or 4.

In some embodiments, the compound is enriched for the atropisomer of formula (IIA-iii):

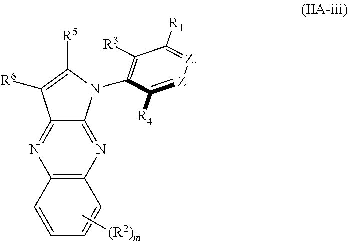

In some embodiments, the compound is of formula (IIA-iv):

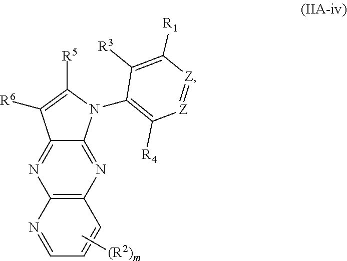

where m is 0, 1, 2, or 3.

In some embodiments, the compound is enriched for the atropisomer of formula (IIA-v):

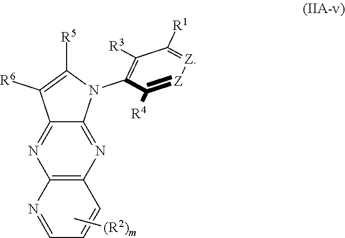

In some embodiments, the compound is of formula (IIA-vi):

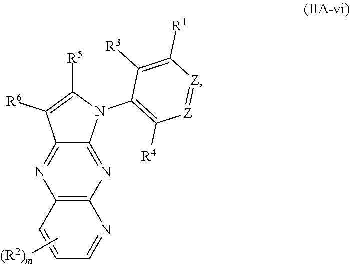

where m is 0, 1, 2, or 3.

In some embodiments, the compound is enriched for the atropisomer of formula (IIA-vii):

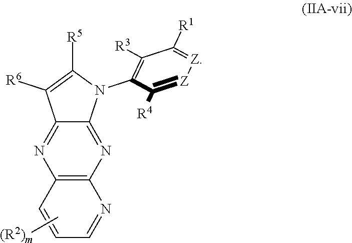

In some embodiments, the compound is of formula (IIB):

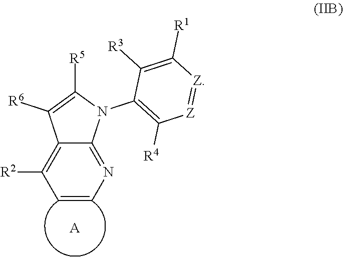

In some embodiments, the compound is enriched for the atropisomer of formula (IIB-i):

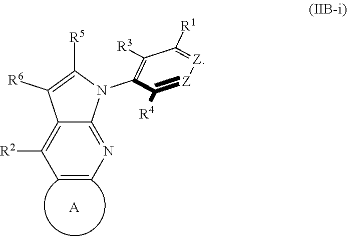

In some embodiments, the compound is of formula (IIB-ii):

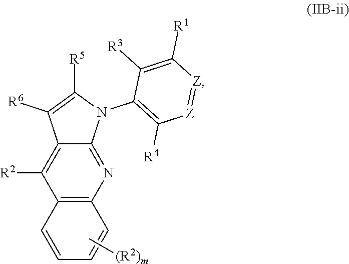

where m is 0, 1, 2, 3, or 4.

In some embodiments, the compound is enriched for the atropisomer of formula (IIB-iii):

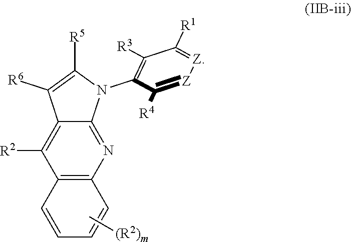

In some embodiments, the compound is of formula (IIB-iv):

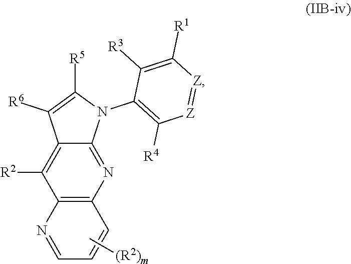

where m is 0, 1, 2, or 3.

In some embodiments, the compound is enriched for the atropisomer of formula (IIB-v):

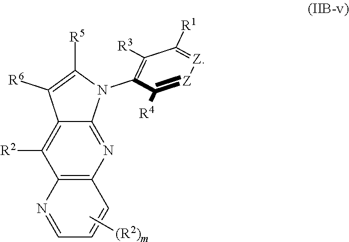

In some embodiments, the compound is of formula (IIB-vi):

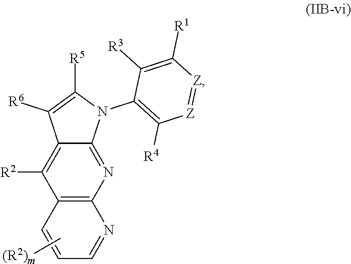

where m is 0, 1, 2, or 3.

In some embodiments, the compound is enriched for the atropisomer of formula (IIB-vii):

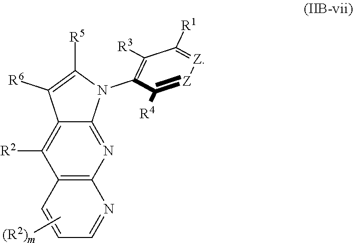

In some embodiments, the compound is of formula (IIC):

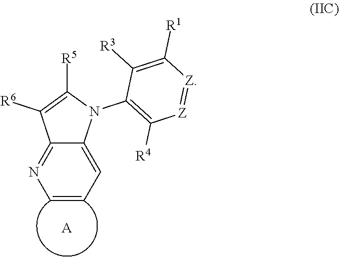

In some embodiments, the compound is enriched for the atropisomer of formula (IIC-i):

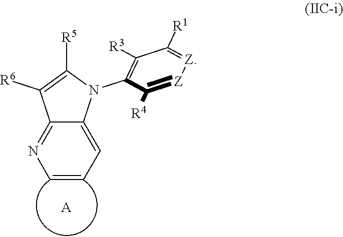

In some embodiments, the compound is of formula (IIC-ii):

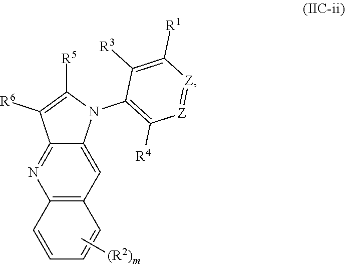

where m is 0, 1, 2, 3, or 4.

In some embodiments, the compound is enriched for the atropisomer of formula (IIC-iii):

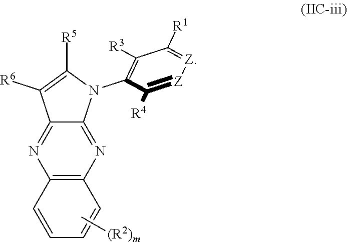

In some embodiments, the compound is of formula (IIC-iv):

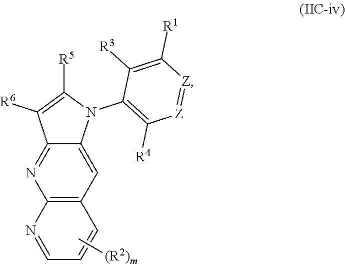

where m is 0, 1, 2, or 3.

In some embodiments, the compound is enriched for the atropisomer of formula (IIC-v):

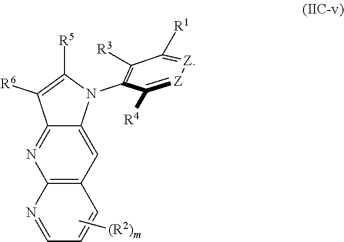

In some embodiments, the compound is of formula (IIC-vi):

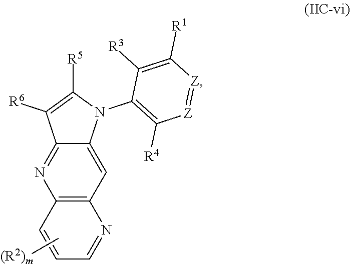

where m is 0, 1, 2, or 3.

In some embodiments, the compound is enriched for the atropisomer of formula (IIC-vii):

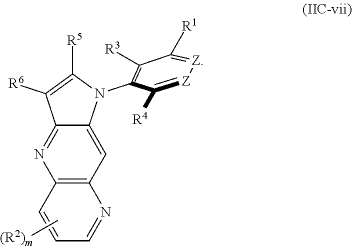

In some embodiments, the compound is of formula (IIC-viii):

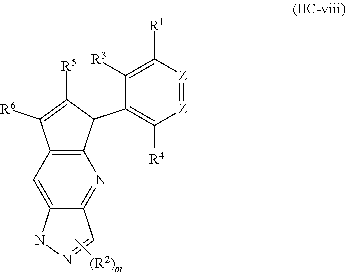

In some embodiments, R² and R^{2A}, together with the atoms to which they are attached, combine to form ring A, and R^{2B} is absent or hydrogen.

In some embodiments, the compound is of formula (III):

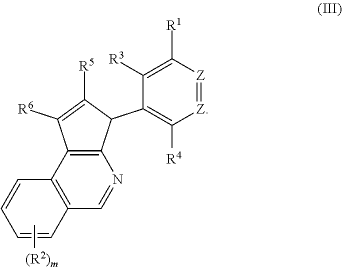

In some embodiments, ring A is a 5- or 6-membered heteroaryl ring that is optionally substituted with 1, 2, 3, or 4 non-hydrogen R² groups. In some embodiments, ring A is a non-aromatic 5- or 6-membered carbocyclic ring that is optionally substituted with 1, 2, 3, or 4 non-hydrogen R² groups.

In some embodiments, Z proximal to R¹ is N. In some embodiments, Z proximal to R¹ is CH. In some embodiments, Z proximal to R⁴ is N. In some embodiments, Z proximal to R⁴ is CH.

In some embodiments, R³ is optionally substituted C₁₋₆ alkyl. In some embodiments, R³ is halogen. In some embodiments, R¹ is OH. In some embodiments, R¹ and R³ combine to form —CR⁹═N—NH—. In some embodiments, R⁹ is hydrogen. In some embodiments, R⁹ is halogen.

In some embodiments, R⁴ is optionally substituted C₁₋₆ alkyl. In some embodiments, R⁴ is halogen.

In some embodiments, one or two R² groups are independently halogen. In some embodiments, one or two R² is optionally substituted C₁₋₆ alkyl. In some embodiments, one or two R² are independently methyl, 3-hydroxypropyl, 2-hydroxyprop-2-yl, methoxycarbonyl, hydroxycarbonyl, aminocarbonyl, N,N-dimethylaminocarbonyl, or N-ethylaminocarbonyl. In some embodiments, one or two R² is optionally substituted C₂₋₆ alkenyl. In some embodiments, one or two R² is 2-propenyl. In some embodiments, one R² is optionally substituted heteroaryl. In some embodiments, one R² is 1-methylpyrazolyl, pyrazolyl, or pyridyl. In some embodiments, one R² is optionally substituted C₃₋₈ cycloalkenyl. In some embodiments, R² is cyclopentenyl. In some embodiments, one or two R² are halogen. In some embodiments, each R² is independently bromine, chlorine, or fluorine. In some embodiments, one or two R² are independently -Q-R^{7B}. In some embodiments, one or two Q are independently optionally substituted C₂₋₆ alkynylene. In some embodiments, one or two R² are independently cyclopropylethynyl, pyrazinylethynyl, 3-(N-morpholinyl)propynyl, or 3-hydroxypropynyl. In some embodiments, Q is optionally substituted C₆₋₁₀ arylene. In some embodiments, Q is phenylene. In some embodiments, R^{7B} is hydrogen, cyano, or N-(2-hydroxyethyl)-N′-piperazinyl. In some embodiments, one R² is optionally substituted C₆₋₁₀ aryl. In some embodiments, one R² is phenyl, cyanophenyl, or [N-(2-hydroxyethyl)-N′-piperazinyl]phenyl. In some embodiments, one or two R² are —C(O)N(R⁸)₂. In some embodiments, each R⁸ is independently hydrogen, methyl, ethyl, N-morpholinylcarbonyl, 3-hydroxypropylaminocarbonyl, or N-methylpiperazinylcabronyl. In some embodiments, one R² is cyano. In some embodiments, one R² is optionally substituted C₂₋₉ heterocyclyl. In some embodiments, one R² is dihydropyranyl.

In some embodiments, m is 4. In some embodiments, m is 3. In some embodiments, m is 2. In some embodiments, m is 1. In some embodiments, m is 0.

In some embodiments, R⁵ is hydrogen. In some embodiments, R⁵ is —N(R⁷)₂. In some embodiments, R⁵ is —NH₂. In some embodiments, R⁵ is halogen. In some embodiments, R⁵ is chlorine.

In some embodiments, R⁶ is —C(O)NH(R⁸). In some embodiments, R⁶ is —C(O)NH₂.

In some embodiments, the compound is not compound 84.

In some embodiments, the compound is selected from the group consisting of compounds 1-89 and pharmaceutically acceptable salts thereof. In some embodiments, the compound is selected from the group consisting of compounds 1-85 and pharmaceutically acceptable salts thereof. In some embodiments, the compound is selected from the group consisting of compounds 1-83, 85, and pharmaceutically acceptable salts thereof.

In another aspect, the invention provides a pharmaceutical composition including the compound disclosed herein, or a pharmaceutically acceptable salt thereof, and a pharmaceutically acceptable excipient. In some embodiments, the composition is isotopically enriched in deuterium.

In yet another aspect, the invention provides a method of inhibiting Myt1 in a cell expressing Myt1, the method including contacting the cell with the compound disclosed herein.

In some embodiments, the cell is overexpressing CCNE1. In some embodiments, the cell is in a subject.

In still another aspect, the invention provides a method of treating a subject in need thereof including administering to the subject the compound disclosed herein, or a pharmaceutically acceptable salt thereof, or the pharmaceutical composition disclosed herein.

In some embodiments, the subject is suffering from, and is in need of a treatment for, a disease or condition having the symptom of cell hyperproliferation. In some embodiments, the disease or condition is a cancer. In some embodiments, the cancer is a cancer overexpressing CCNE1.

In still another aspect, the invention provides a method of treating a cancer in a subject, the method including administering to the subject in need thereof a therapeutically effective amount of a Myt1 inhibitor, where the cancer has been previously identified as a cancer overexpressing CCNE1.

In another aspect, the invention provides a method of treating a cancer in a subject, the method including administering to the subject in need thereof a therapeutically effective amount of a Myt1 inhibitor, where the cancer is a cancer overexpressing CCNE1.

In yet another aspect, the invention provides a method of inducing cell death in a cancer cell overexpressing CCNE1, the method including contacting the cell with an effective amount of a Myt1 inhibitor.

In some embodiments, the cell is in a subject. In some embodiments, the Myt1 inhibitor is the compound disclosed herein or a pharmaceutically acceptable salt thereof. In some embodiments, the cancer overexpressing CCNE1 is uterine cancer, ovarian cancer, breast cancer, stomach cancer, esophageal cancer, lung cancer, or endometrial cancer.

In still another aspect, the invention provides a method of treating a cancer in a subject, the method including administering to the subject in need thereof a therapeutically effective amount of a Myt1 inhibitor, where the cancer has been previously identified as a cancer having an inactivating mutation in the FBXW7 gene.

In another aspect, the invention provides a method of treating a cancer in a subject, the method including administering to the subject in need thereof a therapeutically effective amount of a Myt1 inhibitor, where the cancer has an inactivating mutation in the FBXW7 gene.

In yet another aspect, the invention provides a method of inducing cell death in an FBXW7-mutated cancer cell, the method including contacting the cell with an effective amount of a Myt1 inhibitor.

In some embodiments, the cell is in a subject. the cancer is uterine cancer, colorectal cancer, breast cancer, lung cancer, or esophageal cancer. In some embodiments, the Myt1 inhibitor is the compound disclosed herein, or a pharmaceutically acceptable salt thereof.

### Abbreviations

Abbreviations and terms that are commonly used in the fields of organic chemistry, medicinal chemistry, pharmacology, and medicine and are well known to practitioners in these fields are used herein. Representative abbreviations and definitions are provided below:
Ac is acetyl [CH₃C(O)—];
ACN is acetonitrile;
Ac₂O is acetic anhydride;
AcOH is acetic acid;
APC is antigen-presenting cell;
Ar is aryl;
aq. is aqueous;
9-BBN is 9-borabicyclo[3.3.1]nonane;
BINAP is (2,2′-bis(diphenylphosphino)-1,1′-binaphthyl);
Bn is benzyl;
Boc is tert Butyloxycarbonyl;
n-BuLi is n-butyl lithium;
CDI is carbonyldiimidazole;
cmpd is compound;
conc. is concentrated;
DCM is dichloromethane;
DIAD is diisopropylazodicarboxylate;
DIBAL is diisobutylaluminum hydride;
DIPEA is diisoproplyethyl amine;
DMA is dimethylacetamide;
DMAP is 4-dimethylaminopyridine;
DME is dimethoxyethane;
DMF is N,N-dimethylformamide;
DMSO is dimethyl sulfoxide;
dppf is 1,1′-bis(diphenylphosphino)ferrocene;
EDAC (or EDC) is 1-ethyl-3-[3-(dimethylamino)propyl]-carbodiimide HCl;
ESI is electrospray ionization mass spectrometry;
Et₂O is diethyl ether;
Et₃N is triethylamine;
Et is ethyl;
EtOAc is ethyl acetate;
EtOH is ethanol;
3-F-Ph is 3-fluorophenyl,
h is hour;
HATU is (1-[bis(dimethylamino)methylene]-1H-1,2,3-triazolo[4,5-b]pyridinium 3-oxide hexafluorophosphate;
HCl is hydrochloric acid;
Hex is hexanes;
HOBt is 1-hydroxybenzotriazole;
HPLC is high performance liquid chromatography;
IPA is isopropanol;
LCMS is HPLC with mass spectral detection;
LiHMDS is lithium bis(trimethylsilyl)amide;
LG is leaving group;
M is molar;
mCPBA is metachloroperbenzoic acid;
mmol is millimole;
Me is methyl;
MeCN is acetonitrile;
MeMgBr is methylmagnesium bromide;
MeMgCI is methylmagnesium chloride;
MeOH is methanol;
min is minute;
MOM is methoxymethyl;
Ms is methanesulfonyl;
MS is mass spectrometry;
MW is microwave;
N is normal;
NaHMDS is sodium bis(trimethylsilyl)amide;
NaOAc is sodium acetate;
NaOtBu is sodium tert-butoxide;
NBS is N-bromosuccinimide;
NCS is N-chlorosuccinimide;
NIS is N-iodosuccinimide;
NMO is N-methylmorpholine N-oxide;
NMP is N-methyl pyrrolidinone;
NMR is nuclear magnetic resonance spectroscopy;
PdCl₂(dppf) is [1,1′-Bis(diphenylphosphino)ferrocene]dichloropalladium(II);
PdCl₂(dppf.CH₂Cl₂ is [1,1′-Bis(diphenylphosphino)ferrocene]dichloropalladium(II), complex with dichloromethane;
Pd₂(dba)₃ is tris(dibenzylideneacetone)dipalladium;
PdCl₂(PPh₃)₂ is dichlorobis-(triphenylphosphene) palladium;
Pd-PEPPSI™-SIPr is (1,3-Bis(2,6-diisopropylphenyl)imidazolidene) (3-chloropyridyl) palladium(II) dichloride;
PG Denotes a protecting group;
Ph is phenyl;
PhMe is toluene;
PIV-Cl is pivaloyl chloride, Trimethylacetyl chloride;
PPh₃ is triphenylphosphine;
PMB is para-methoxybenzyl;
rt or RT is room temperature;
RBF is round-bottom flask;
RuPhos Pd G1 is chloro-(2-Dicyclohexylphosphino-2′,6′-diisopropoxy-1,1′-biphenyl)[2-(2-aminoethyl)phenyl]palladium(II);
sat. is saturated;
SEM is [2-(trimethylsilyl)ethoxy]methyl;
SFC is supercritical fluid chromatography;
S_{N}Ar is nucleophilic aromatic substitution;
TBAB is tetrabutyl ammonium bromide;
TBAF is tetrabutyl ammonium fluoride;
TBS is tert-butyldimethylsilyl;
tBu is tert-butyl;
Tf is trifluoromethanesulfonyl;
TFA is trifluoroacetic acid;
THE is tetrahydrofuran;
THP is tetrahydropyran;
TLC is thin layer chromatography;
TMAD is tetramethylazodicarboxamide;
TMS is trimethylsilyl;
TPAP is tetrapropylammonium perruthenate;
Ts is p-toluenesulfonyl;
UPLC is ultra-performance liquid chromatography;
Xantphos is 4,5-Bis(diphenylphosphino)-9,9-dimethylxanthene.

### Definitions

The term “aberrant,” as used herein, refers to different from normal. When used to describe activity, aberrant refers to activity that is greater or less than a normal control or the average of normal non-diseased control samples. Aberrant activity may refer to an amount of activity that results in a disease, where returning the aberrant activity to a normal or non-disease-associated amount (e.g. by administering a compound or using a method as described herein), results in reduction of the disease or one or more disease symptoms.

The term “acyl,” as used herein, represents a group —C(═O)—R, where R is alkyl, alkenyl, alkynyl, cycloalkyl, cycloalkenyl, cycloalkynyl, aryl, heteroaryl, or heterocyclyl. Acyl may be optionally substituted as described herein for each respective R group.

The term “adenocarcinoma,” as used herein, represents a malignancy of the arising from the glandular cells that line organs within an organism. Non-limiting examples of adenocarcinomas include non-small cell lung cancer, prostate cancer, pancreatic cancer, esophageal cancer, and colorectal cancer.

The term “alkanoyl,” as used herein, represents a hydrogen or an alkyl group that is attached to the parent molecular group through a carbonyl group and is exemplified by formyl (i.e., a carboxyaldehyde group), acetyl, propionyl, butyryl, and iso-butyryl. Unsubstituted alkanoyl groups contain from 1 to 7 carbons. The alkanoyl group may be unsubstituted of substituted (e.g., optionally substituted C₁₋₇ alkanoyl) as described herein for alkyl group. The ending “-oyl” may be added to another group defined herein, e.g., aryl, cycloalkyl, and heterocyclyl, to define “aryloyl,” “cycloalkanoyl,” and “(heterocyclyl)oyl.” These groups represent a carbonyl group substituted by aryl, cycloalkyl, or heterocyclyl, respectively. Each of “aryloyl,” “cycloalkanoyl,” and “(heterocyclyl)oyl” may be optionally substituted as defined for “aryl,” “cycloalkyl,” or “heterocyclyl,” respectively.

The term “alkenyl,” as used herein, represents acyclic monovalent straight or branched chain hydrocarbon groups of containing one, two, or three carbon-carbon double bonds. Non-limiting examples of the alkenyl groups include ethenyl, prop-1-enyl, prop-2-enyl, 1-methylethenyl, but-1-enyl, but-2-enyl, but-3-enyl, 1-methylprop-1-enyl, 2-methylprop-1-enyl, and 1-methylprop-2-enyl. Alkenyl groups may be optionally substituted as defined herein for alkyl.

The term “alkenylene,” as used herein, refers to a divalent alkenyl group. An optionally substituted alkenylene is an alkenylene that is optionally substituted as described herein for alkenyl.

The term “alkoxy,” as used herein, represents a chemical substituent of formula —OR, where R is a C₁₋₆ alkyl group, unless otherwise specified. In some embodiments, the alkyl group can be further substituted as defined herein. The term “alkoxy” can be combined with other terms defined herein, e.g., aryl, cycloalkyl, or heterocyclyl, to define an “aryl alkoxy,” “cycloalkyl alkoxy,” and “(heterocyclyl)alkoxy” groups. These groups represent an alkoxy that is substituted by aryl, cycloalkyl, or heterocyclyl, respectively. Each of “aryl alkoxy,” “cycloalkyl alkoxy,” and “(heterocyclyl)alkoxy” may optionally substituted as defined herein for each individual portion.

The term “alkoxyalkyl,” as used herein, represents a chemical substituent of formula -L-O—R, where L is C₁₋₆ alkylene, and R is C₁₋₆ alkyl. An optionally substituted alkoxyalkyl is an alkoxyalkyl that is optionally substituted as described herein for alkyl.

The term “alkyl,” as used herein, refers to an acyclic straight or branched chain saturated hydrocarbon group, which, when unsubstituted, has from 1 to 12 carbons, unless otherwise specified. In certain preferred embodiments, unsubstituted alkyl has from 1 to 6 carbons. Alkyl groups are exemplified by methyl; ethyl; n- and iso-propyl; n-, sec-, iso- and tert-butyl; neopentyl, and the like, and may be optionally substituted, valency permitting, with one, two, three, or, in the case of alkyl groups of two carbons or more, four or more substituents independently selected from the group consisting of: amino; alkoxy; aryl; aryloxy; azido; cycloalkyl; cycloalkoxy; cycloalkenyl; cycloalkynyl; halo; heterocyclyl; (heterocyclyl)oxy; heteroaryl; hydroxy; nitro; thiol; silyl; cyano; alkylsulfonyl; alkylsulfinyl; alkylsulfenyl; ═O; ═S; —C(O)R or —SO₂R, where R is amino; and ═NR′, where R′ is H, alkyl, aryl, or heterocyclyl. Each of the substituents may itself be unsubstituted or, valency permitting, substituted with unsubstituted substituent(s) defined herein for each respective group.

The term “alkylene,” as used herein, refers to a divalent alkyl group. An optionally substituted alkylene is an alkylene that is optionally substituted as described herein for alkyl.

The term “alkylamino,” as used herein, refers to a group having the formula —N(R^{N1})₂ or —NHR^{N1} in which R^{N1} is alkyl, as defined herein. The alkyl portion of alkylamino can be optionally substituted as defined for alkyl. Each optional substituent on the substituted alkylamino may itself be unsubstituted or, valency permitting, substituted with unsubstituted substituent(s) defined herein for each respective group.

The term “alkylsulfenyl,” as used herein, represents a group of formula —S-(alkyl). Alkylsulfenyl may be optionally substituted as defined for alkyl.

The term “alkylsulfinyl,” as used herein, represents a group of formula —S(O)-(alkyl). Alkylsulfinyl may be optionally substituted as defined for alkyl.

The term “alkylsulfonyl,” as used herein, represents a group of formula —S(O)₂-(alkyl).

Alkylsulfonyl may be optionally substituted as defined for alkyl.

The term “alkynyl,” as used herein, represents monovalent straight or branched chain hydrocarbon groups of from two to six carbon atoms containing at least one carbon-carbon triple bond and is exemplified by ethynyl, 1-propynyl, and the like. The alkynyl groups may be unsubstituted or substituted (e.g., optionally substituted alkynyl) as defined for alkyl.

The term “alkynylene,” as used herein, refers to a divalent alkynyl group. An optionally substituted alkynylene is an alkynylene that is optionally substituted as described herein for alkynyl.

The term “amino,” as used herein, represents —N(R^{N1})₂, where, if amino is unsubstituted, both R^{N1} are H; or, if amino is substituted, each R^{N1} is independently H, —OH, —NO₂, —N(R^{N2})₂, —SO₂OR^{N2}, —SO₂R^{N2}, —SOR^{N2}, —C(O)OR^{N2}, an N-protecting group, alkyl, alkenyl, alkynyl, alkoxy, aryl, arylalkyl, aryloxy, cycloalkyl, cycloalkenyl, heteroalkyl, or heterocyclyl, provided that at least one R^{N1} is not H, and where each R^{N2} is independently H, alkyl, or aryl. Each of the substituents may itself be unsubstituted or substituted with unsubstituted substituent(s) defined herein for each respective group. In some embodiments, amino is unsubstituted amino (i.e., —NH₂) or substituted amino (e.g., —NHR^{N1}), where R^{N1} is independently —OH, SO₂OR^{N2}, —SO₂R^{N2}, —SOR^{N2}, —COOR^{N2}, optionally substituted alkyl, or optionally substituted aryl, and each R^{N2} can be optionally substituted alkyl or optionally substituted aryl. In some embodiments, substituted amino may be alkylamino, in which the alkyl groups are optionally substituted as described herein for alkyl. In some embodiments, an amino group is —NHR^{N1}, in which R^{N1} is optionally substituted alkyl.

The term “aryl,” as used herein, represents a mono-, bicyclic, or multicyclic carbocyclic ring system having one or two aromatic rings. Aryl group may include from 6 to 10 carbon atoms. All atoms within an unsubstituted carbocyclic aryl group are carbon atoms. Non-limiting examples of carbocyclic aryl groups include phenyl, naphthyl, 1,2-dihydronaphthyl, 1,2,3,4-tetrahydronaphthyl, fluorenyl, indanyl, indenyl, etc. The aryl group may be unsubstituted or substituted with one, two, three, four, or five substituents independently selected from the group consisting of: alkyl; alkenyl; alkynyl; alkoxy; alkylsulfinyl; alkylsulfenyl; alkylsulfonyl; amino; aryl; aryloxy; azido; cycloalkyl; cycloalkoxy; cycloalkenyl; cycloalkynyl; halo; heteroalkyl; heterocyclyl; (heterocyclyl)oxy; hydroxy; nitro; thiol; silyl; —(CH₂)ₙ—C(O)OR^{A}; —C(O)R; and —SO₂R, where R is amino or alkyl, R^{A} is H or alkyl, and n is 0 or 1. Each of the substituents may itself be unsubstituted or substituted with unsubstituted substituent(s) defined herein for each respective group.

The term “aryl alkyl,” as used herein, represents an alkyl group substituted with an aryl group. The aryl and alkyl portions may be optionally substituted as the individual groups as described herein.

The term “arylene,” as used herein, refers to a divalent aryl group. An optionally substituted arylene is an arylene that is optionally substituted as described herein for aryl.

The term “aryloxy,” as used herein, represents a chemical substituent of formula —OR, where R is an aryl group, unless otherwise specified. In optionally substituted aryloxy, the aryl group is optionally substituted as described herein for aryl.

The term “azido,” as used herein, represents an —N₃ group.

The term “cancer,” as used herein, refers to all types of cancer, neoplasm or malignant tumors found in mammals (e.g., humans).

The term “carbocyclic,” as used herein, represents an optionally substituted C₃₋₁₆ monocyclic, bicyclic, or tricyclic structure in which the rings, which may be aromatic or non-aromatic, are formed by carbon atoms. Carbocyclic structures include cycloalkyl, cycloalkenyl, cycloalkynyl, and certain aryl groups.

The term “carbonyl,” as used herein, represents a —C(O)— group.

The term “carcinoma,” as used herein, refers to a malignant new growth made up of epithelial cells tending to infiltrate the surrounding tissues and give rise to metastases.

The term “cyano,” as used herein, represents —CN group.

The terms “CCNE1” and “cyclin E1,” as used interchangeably herein, refer to G1/S specific cyclin E1 (Gene name: CCNE1). A cell overexpressing CCNE1 is one that exhibits a higher activity of CCNE1 than a cell normally expressing CCNE1. For example, a CCNE1-overexpressing cell is a cell that exhibits a copy number of at least 3 compared to a diploid normal cell with 2 copies. Thus, a cell exhibiting a copy number greater than 3 of CCNE1 is a cell overexpressing CCNE1. The CCNE1 overexpression may be measured by identifying the expression level of the gene product in a cell (e.g., CCNE1 mRNA transcript count or CCNE1 protein level).

The term “cycloalkenyl,” as used herein, refers to a non-aromatic carbocyclic group having at least one double bond in the ring and from three to ten carbons (e.g., a C₃₋₁₀ cycloalkenyl), unless otherwise specified. Non-limiting examples of cycloalkenyl include cycloprop-1-enyl, cycloprop-2-enyl, cyclobut-1-enyl, cyclobut-1-enyl, cyclobut-2-enyl, cyclopent-1-enyl, cyclopent-2-enyl, cyclopent-3-enyl, norbornen-1-yl, norbornen-2-yl, norbornen-5-yl, and norbornen-7-yl. The cycloalkenyl group may be unsubstituted or substituted (e.g., optionally substituted cycloalkenyl) as described for cycloalkyl.

The term “cycloalkenyl alkyl,” as used herein, represents an alkyl group substituted with a cycloalkenyl group, each as defined herein. The cycloalkenyl and alkyl portions may be substituted as the individual groups defined herein.

The term “cycloalkenylene,” as used herein, represents a divalent cycloalkenyl group. An optionally substituted cycloalkenylene is a cycloalkenylene that is optionally substituted as described herein for cycloalkyl.

The term “cycloalkoxy,” as used herein, represents a chemical substituent of formula —OR, where R is cycloalkyl group, unless otherwise specified. In some embodiments, the cycloalkyl group can be further substituted as defined herein.

The term “cycloalkyl,” as used herein, refers to a cyclic alkyl group having from three to ten carbons (e.g., a C₃c1₀ cycloalkyl), unless otherwise specified. Cycloalkyl groups may be monocyclic or bicyclic. Bicyclic cycloalkyl groups may be of bicyclo[p.q.0]alkyl type, in which each of p and q is, independently, 1, 2, 3, 4, 5, 6, or 7, provided that the sum of p and q is 2, 3, 4, 5, 6, 7, or 8. Alternatively, bicyclic cycloalkyl groups may include bridged cycloalkyl structures, e.g., bicyclo[p.q.r]alkyl, in which r is 1, 2, or 3, each of p and q is, independently, 1, 2, 3, 4, 5, or 6, provided that the sum of p, q, and r is 3, 4, 5, 6, 7, or 8. The cycloalkyl group may be a spirocyclic group, e.g., spiro[p.q]alkyl, in which each of p and q is, independently, 2, 3, 4, 5, 6, or 7, provided that the sum of p and q is 4, 5, 6, 7, 8, or 9. Non-limiting examples of cycloalkyl include cyclopropyl, cyclobutyl, cyclopentyl, cyclohexyl, cycloheptyl, 1-bicyclo[2.2.1.]heptyl, 2-bicyclo[2.2.1.]heptyl, 5-bicyclo[2.2.1.]heptyl, 7-bicyclo[2.2.1.]heptyl, and decalinyl. The cycloalkyl group may be unsubstituted or substituted (e.g., optionally substituted cycloalkyl) with one, two, three, four, or five substituents independently selected from the group consisting of: alkyl; alkenyl; alkynyl; alkoxy; alkylsulfinyl; alkylsulfenyl; alkylsulfonyl; amino; aryl; aryloxy; azido; cycloalkyl; cycloalkoxy; cycloalkenyl; cycloalkynyl; halo; heteroalkyl; heterocyclyl; (heterocyclyl)oxy; heteroaryl; hydroxy; nitro; thiol; silyl; cyano; ═O; ═S; —SO₂R, where R is optionally substituted amino; ═NR′, where R¹ is H, alkyl, aryl, or heterocyclyl; and —CON(R^{A})₂, where each R^{A} is independently H or alkyl, or both R^{A}, together with the atom to which they are attached, combine to form heterocyclyl. Each of the substituents may itself be unsubstituted or substituted with unsubstituted substituent(s) defined herein for each respective group.

The term “cycloalkyl alkyl,” as used herein, represents an alkyl group substituted with a cycloalkyl group, each as defined herein. The cycloalkyl and alkyl portions may be optionally substituted as the individual groups described herein.

The term “cycloalkylene,” as used herein, represents a divalent cycloalkyl group. An optionally substituted cycloalkylene is a cycloalkylene that is optionally substituted as described herein for cycloalkyl.

The term “cycloalkynyl,” as used herein, refers to a monovalent carbocyclic group having one or two carbon-carbon triple bonds and having from eight to twelve carbons, unless otherwise specified. Cycloalkynyl may include one transannular bond or bridge. Non-limiting examples of cycloalkynyl include cyclooctynyl, cyclononynyl, cyclodecynyl, and cyclodecadiynyl. The cycloalkynyl group may be unsubstituted or substituted (e.g., optionally substituted cycloalkynyl) as defined for cycloalkyl.

“Disease” or “condition” refer to a state of being or health status of a patient or subject capable of being treated with the compounds or methods provided herein.

The term “FBXW7,” as used herein, refers to F-box/WD Repeat-Containing Protein 7 gene, transcript, or protein. An FBXW7-mutated gene, also described herein as an FBXW7 gene having an inactivating mutation, is one that fails to produce a functional FBXW7 protein or produces reduced quantities of FBXW7 protein in a cell.

The term “halo,” as used herein, represents a halogen selected from bromine, chlorine, iodine, and fluorine.

The term “heteroalkyl,” as used herein refers to an alkyl, alkenyl, or alkynyl group interrupted once by one or two heteroatoms; twice, each time, independently, by one or two heteroatoms; three times, each time, independently, by one or two heteroatoms; or four times, each time, independently, by one or two heteroatoms. Each heteroatom is, independently, O, N, or S. In some embodiments, the heteroatom is O or N. None of the heteroalkyl groups includes two contiguous oxygen or sulfur atoms.

The heteroalkyl group may be unsubstituted or substituted (e.g., optionally substituted heteroalkyl).

When heteroalkyl is substituted and the substituent is bonded to the heteroatom, the substituent is selected according to the nature and valency of the heteratom. Thus, the substituent bonded to the heteroatom, valency permitting, is selected from the group consisting of ═O, —N(R^{N2})₂, —SO₂OR^{N3}, —SO₂R^{N2}, —SOR^{N3}, —COOR^{N3}, an N protecting group, alkyl, alkenyl, alkynyl, aryl, cycloalkyl, cycloalkenyl, cycloalkynyl, heterocyclyl, or cyano, where each R^{N2} is independently H, alkyl, cycloalkyl, cycloalkenyl, cycloalkynyl, aryl, or heterocyclyl, and each R^{N3} is independently alkyl, cycloalkyl, cycloalkenyl, cycloalkynyl, aryl, or heterocyclyl. Each of these substituents may itself be unsubstituted or substituted with unsubstituted substituent(s) defined herein for each respective group. When heteroalkyl is substituted and the substituent is bonded to carbon, the substituent is selected from those described for alkyl, provided that the substituent on the carbon atom bonded to the heteroatom is not Cl, Br, or I. It is understood that carbon atoms are found at the termini of a heteroalkyl group.

The term “heteroaryl alkyl,” as used herein, represents an alkyl group substituted with a heteroaryl group, each as defined herein. The heteroaryl and alkyl portions may be optionally substituted as the individual groups described herein.

The term “heteroarylene,” as used herein, represents a divalent heteroaryl. An optionally substituted heteroarylene is a heteroarylene that is optionally substituted as described herein for heteroaryl.

The term “heteroaryloxy,” as used herein, refers to a structure —OR, in which R is heteroaryl. Heteroaryloxy can be optionally substituted as defined for heterocyclyl.

The term “heterocyclyl,” as used herein, represents a monocyclic, bicyclic, tricyclic, or tetracyclic ring system having fused, bridging, and/or spiro 3-, 4-, 5-, 6-, 7-, or 8-membered rings, unless otherwise specified, containing one, two, three, or four heteroatoms independently selected from the group consisting of nitrogen, oxygen, and sulfur. In some embodiments, “heterocyclyl” is a monocyclic, bicyclic, tricyclic, or tetracyclic ring system having fused or bridging 5-, 6-, 7-, or 8-membered rings, unless otherwise specified, containing one, two, three, or four heteroatoms independently selected from the group consisting of nitrogen, oxygen, and sulfur. Heterocyclyl can be aromatic or non-aromatic. Non-aromatic 5-membered heterocyclyl has zero or one double bonds, non-aromatic 6- and 7-membered heterocyclyl groups have zero to two double bonds, and non-aromatic 8-membered heterocyclyl groups have zero to two double bonds and/or zero or one carbon-carbon triple bond. Heterocyclyl groups include from 1 to 16 carbon atoms unless otherwise specified. Certain heterocyclyl groups may include up to 9 carbon atoms. Non-aromatic heterocyclyl groups include pyrrolinyl, pyrrolidinyl, pyrazolinyl, pyrazolidinyl, imidazolinyl, imidazolidinyl, piperidinyl, homopiperidinyl, piperazinyl, pyridazinyl, oxazolidinyl, isoxazolidiniyl, morpholinyl, thiomorpholinyl, thiazolidinyl, isothiazolidinyl, thiazolidinyl, tetrahydrofuranyl, dihydrofuranyl, tetrahydrothienyl, dihydrothienyl, dihydroindolyl, tetrahydroquinolyl, tetrahydroisoquinolyl, pyranyl, dihydropyranyl, dithiazolyl, etc. If the heterocyclic ring system has at least one aromatic resonance structure or at least one aromatic tautomer, such structure is an aromatic heterocyclyl (i.e., heteroaryl). Non-limiting examples of heteroaryl groups include benzimidazolyl, benzofuryl, benzothiazolyl, benzothienyl, benzoxazolyl, furyl, imidazolyl, indolyl, isoindazolyl, isoquinolinyl, isothiazolyl, isothiazolyl, isoxazolyl, oxadiazolyl, oxazolyl, purinyl, pyrrolyl, pyridinyl, pyrazinyl, pyrimidinyl, qunazolinyl, quinolinyl, thiadiazolyl (e.g., 1,3,4-thiadiazole), thiazolyl, thienyl, triazolyl, tetrazolyl, etc. The term “heterocyclyl” also represents a heterocyclic compound having a bridged multicyclic structure in which one or more carbons and/or heteroatoms bridges two non-adjacent members of a monocyclic ring, e.g., quinuclidine, tropanes, or diaza-bicyclo[2.2.2]octane. The term “heterocyclyl” includes bicyclic, tricyclic, and tetracyclic groups in which any of the above heterocyclic rings is fused to one, two, or three carbocyclic rings, e.g., an aryl ring, a cyclohexane ring, a cyclohexene ring, a cyclopentane ring, a cyclopentene ring, or another monocyclic heterocyclic ring. Examples of fused heterocyclyls include 1,2,3,5,8,8a-hexahydroindolizine; 2,3-dihydrobenzofuran; 2,3-dihydroindole; and 2,3-dihydrobenzothiophene. The heterocyclyl group may be unsubstituted or substituted with one, two, three, four or five substituents independently selected from the group consisting of: alkyl; alkenyl; alkynyl; alkoxy; alkylsulfinyl; alkylsulfenyl; alkylsulfonyl; amino; aryl; aryloxy; azido; cycloalkyl; cycloalkoxy; cycloalkenyl; cycloalkynyl; halo; heteroalkyl; heterocyclyl; (heterocyclyl)oxy; hydroxy; nitro; thiol; silyl; cyano; —C(O)R or —SO₂R, where R is amino or alkyl; ═O; ═S; ═NR′, where R′ is H, alkyl, aryl, or heterocyclyl. Each of the substituents may itself be unsubstituted or substituted with unsubstituted substituent(s) defined herein for each respective group.

The term “heterocyclyl alkyl,” as used herein, represents an alkyl group substituted with a heterocyclyl group, each as defined herein. The heterocyclyl and alkyl portions may be optionally substituted as the individual groups described herein.

The term “heterocyclylene,” as used herein, represents a divalent heterocyclyl. An optionally substituted heterocyclylene is a heterocyclylene that is optionally substituted as described herein for heterocyclyl.

The term “(heterocyclyl)oxy,” as used herein, represents a chemical substituent of formula —OR, where R is a heterocyclyl group, unless otherwise specified. (Heterocyclyl)oxy can be optionally substituted in a manner described for heterocyclyl.

The terms “hydroxyl” and “hydroxy,” as used interchangeably herein, represent an —OH group.

The term “isotopically enriched,” as used herein, refers to the pharmaceutically active agent with the isotopic content for one isotope at a predetermined position within a molecule that is at least 100 times greater than the natural abundance of this isotope. For example, a composition that is isotopically enriched for deuterium includes an active agent with at least one hydrogen atom position having at least 100 times greater abundance of deuterium than the natural abundance of deuterium. Preferably, an isotopic enrichment for deuterium is at least 1000 times greater than the natural abundance of deuterium. More preferably, an isotopic enrichment for deuterium is at least 4000 times greater (e.g., at least 4750 times greater, e.g., up to 5000 times greater) than the natural abundance of deuterium.

The term “leukemia,” as used herein, refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic).

The term “lymphoma,” as used herein, refers to a cancer arising from cells of immune origin.

The term “melanoma,” as used herein, is taken to mean a tumor arising from the melanocytic system of the skin and other organs.

The term “Myt1,” as used herein, refers to membrane-associated tyrosine and threonine-specific cdc2-inhibitory kinase (Myt1) (Gene name PKMYT1).

The term “Myt1 inhibitor,” as used herein, represents a compound that upon contacting the enzyme Myt1, whether in vitro, in cell culture, or in an animal, reduces the activity of Myt1, such that the measured Myt1 IC₅₀ is 10 μM or less (e.g., 5 μM or less or 1 μM or less). For certain Myt1 inhibitors, the Myt1 IC₅₀ may be 100 nM or less (e.g., 10 nM or less, or 3 nM or less) and could be as low as 100 μM or 10 μM. Preferably, the Myt1 IC₅₀ is 1 nM to 1 μM (e.g., 1 nM to 750 nM, 1 nM to 500 nM, or 1 nM to 250 nM). Even more preferably, the Myt1 IC₅₀ is less than 20 nm (e.g., 1 nM to 20 nM).

The term “nitro,” as used herein, represents an —NO₂ group.

The term “oxo,” as used herein, represents a divalent oxygen atom (e.g., the structure of oxo may be shown as ═O).

The term “Ph,” as used herein, represents phenyl.

The term “pharmaceutical composition,” as used herein, represents a composition containing a compound described herein, formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

The term “pharmaceutically acceptable excipient” or “pharmaceutically acceptable carrier,” as used interchangeably herein, refers to any ingredient other than the compounds described herein (e.g., a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable salt,” as use herein, represents those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The term “pre-malignant” or “pre-cancerous,” as used herein, refers to a condition that is not malignant but is poised to become malignant.

The term “protecting group,” as used herein, represents a group intended to protect a hydroxy, an amino, or a carbonyl from participating in one or more undesirable reactions during chemical synthesis.

The term “O-protecting group,” as used herein, represents a group intended to protect a hydroxy or carbonyl group from participating in one or more undesirable reactions during chemical synthesis. The term “N-protecting group,” as used herein, represents a group intended to protect a nitrogen containing (e.g., an amino, amido, heterocyclic N—H, or hydrazine) group from participating in one or more undesirable reactions during chemical synthesis. Commonly used O- and N-protecting groups are disclosed in Greene, “Protective Groups in Organic Synthesis,” 3rd Edition (John Wiley & Sons, New York, 1999), which is incorporated herein by reference. Exemplary O- and N-protecting groups include alkanoyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, t-butyldimethylsilyl, tri-iso-propylsilyloxymethyl, 4,4′-dimethoxytrityl, isobutyryl, phenoxyacetyl, 4-isopropylpehenoxyacetyl, dimethylformamidino, and 4-nitrobenzoyl.

Exemplary O-protecting groups for protecting carbonyl containing groups include, but are not limited to: acetals, acylals, 1,3-dithianes, 1,3-dioxanes, 1,3-dioxolanes, and 1,3-dithiolanes.

Other O-protecting groups include, but are not limited to: substituted alkyl, aryl, and aryl-alkyl ethers (e.g., trityl; methylthiomethyl; methoxymethyl; benzyloxymethyl; siloxymethyl; 2,2,2,-trichloroethoxymethyl; tetrahydropyranyl; tetrahydrofuranyl; ethoxyethyl; 1-[2-(trimethylsilyl)ethoxy]ethyl; 2-trimethylsilylethyl; t-butyl ether; p-chlorophenyl, p-methoxyphenyl, p-nitrophenyl, benzyl, p-methoxybenzyl, and nitrobenzyl); silyl ethers (e.g., trimethylsilyl; triethylsilyl; triisopropylsilyl; dimethylisopropylsilyl; t-butyldimethylsilyl; t-butyldiphenylsilyl; tribenzylsilyl; triphenylsilyl; and diphenymethylsilyl); carbonates (e.g., methyl, methoxymethyl, 9-fluorenylmethyl; ethyl; 2,2,2-trichloroethyl; 2-(trimethylsilyl)ethyl; vinyl, allyl, nitrophenyl; benzyl; methoxybenzyl; 3,4-dimethoxybenzyl; and nitrobenzyl).

Other N-protecting groups include, but are not limited to, chiral auxiliaries such as protected or unprotected D, L or D, L-amino acids such as alanine, leucine, phenylalanine, and the like; sulfonyl-containing groups such as benzenesulfonyl, p-toluenesulfonyl, and the like; carbamate forming groups such as benzyloxycarbonyl, p-chlorobenzyloxycarbonyl, p methoxybenzyloxycarbonyl, p-nitrobenzyloxycarbonyl, 2-nitrobenzyloxycarbonyl, p bromobenzyloxycarbonyl, 3,4-dimethoxybenzyloxycarbonyl, 3,5 dimethoxybenzyl oxycarbonyl, 2,4-dimethoxybenzyloxycarbonyl, 4 methoxybenzyloxycarbonyl, 2-nitro-4,5-dimethoxybenzyloxycarbonyl, 3,4,5 trimethoxybenzyloxycarbonyl, 1-(p-biphenylyl)-1-methylethoxycarbonyl, α,α-dimethyl-3,5 dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, aryl-alkyl groups such as benzyl, p-methoxybenzyl, 2,4-dimethoxybenzyl, triphenylmethyl, benzyloxymethyl, and the like, silylalkylacetal groups such as [2-(trimethylsilyl)ethoxy]methyl and silyl groups such as trimethylsilyl, and the like. Useful N-protecting groups are formyl, acetyl, benzoyl, pivaloyl, t-butylacetyl, alanyl, phenylsulfonyl, benzyl, dimethoxybenzyl, [2-(trimethylsilyl)ethoxy]methyl (SEM), tetrahydropyranyl (THP), t-butyloxycarbonyl (Boc), and benzyloxycarbonyl (Cbz).

The term “tautomer” refers to structural isomers that readily interconvert, often by relocation of a proton. Tautomers are distinct chemical species that can be identified by differing spectroscopic characteristics, but generally cannot be isolated individually. Non-limiting examples of tautomers include ketone—enol, enamine—imine, amide—imidic acid, nitroso—oxime, ketene—ynol, and amino acid—ammonium carboxylate.

The term “sarcoma” generally refers to a tumor which is made up of a substance like the embryonic connective tissue and is generally composed of closely packed cells embedded in a fibrillar or homogeneous substance.

The term “subject,” as used herein, represents a human or non-human animal (e.g., a mammal) that is suffering from, or is at risk of, disease or condition, as determined by a qualified professional (e.g., a doctor or a nurse practitioner) with or without known in the art laboratory test(s) of sample(s) from the subject. Preferably, the subject is a human. Non-limiting examples of diseases and conditions include diseases having the symptom of cell hyperproliferation, e.g., a cancer.

“Treatment” and “treating,” as used herein, refer to the medical management of a subject with the intent to improve, ameliorate, stabilize, prevent or cure a disease or condition. This term includes active treatment (treatment directed to improve the disease or condition); causal treatment (treatment directed to the cause of the associated disease or condition); palliative treatment (treatment designed for the relief of symptoms of the disease or condition); preventative treatment (treatment directed to minimizing or partially or completely inhibiting the development of the associated disease or condition); and supportive treatment (treatment employed to supplement another therapy).

### BRIEF DESCRIPTION OF THE DRAWINGS

FIG. 1A is a bar graph showing the CCNE1 amplification/overexpression across tumors sequenced from TCGA PanCancer Atlas.

FIG. 1B is a scatter plot showing the CCNE1 gene expression data from TCGA PanCancer Atlas.

FIG. 2A is a bar graph showing the FBXW7 mutations across tumors sequenced from TCGA PanCancer Atlas.

FIG. 2B is a lollipop graph showing the frequency of FBXW7 mutations across the gene. This graph highlights three common arginine hotspot mutations (R465, R479, and R505) within the third and fourth WD40 repeats that disrupt recognition of the Cyclin E1 substrate and are classified as deleterious.

FIG. 3A is a bar graph showing the results of a proliferation assay using RPE1-hTERT Cas9 TP53−/− and CCNE1-overexpressing clones treated with different doses of compound A.

FIG. 3B is a series of images depicting the results of a clonogenic survival assay using RPE1-hTERT Cas9 TP53−/− and CCNE1-overexpressing clones transduced with PKMYT1 sgRNAs. Infected cells were plated at low density to measure their ability to form colonies of >50 cells. After 10 days of growth, the colonies were stained, imaged, and quantified. The results are normalized to the survival of RPE1-hTERT Cas9 TP53−/− parental and CCNE1-overexpressing clones transduced with a non-targeting LacZ control sgRNA.

FIG. 3C is a line graph showing the results of a proliferation assay using RPE1-hTERT Cas9 TP54−/− and CCNE1-overexpressing clones treated with different doses of compound A.

FIG. 4A is a bar graph showing the results of a clonogenic survival assay using FT282-hTERT TP53^{R175H} and CCNE1-overexpressing cells transduced with PKMYT1 sgRNAs. Infected cells were plated at low density to measure their ability to form colonies of >50 cells. After 10 days of growth, the colonies were stained, imaged, and quantified. The results are normalized to the survival of FT282-hTERT TP53^{R175H} and CCNE1-overexpressing cells transduced with an AAVS1 control sgRNA.

FIG. 4B is a series of images showing of stained colonies described in FIG. 4A.

FIG. 4C is a line graph showing the results of a proliferation assay using FT282-hTERT TP53^{R175H} and CCNE1-overexpressing clones treated with different doses of compound A.

FIGS. 5A, 5B, and 5C show the results of clonogenic survival assays for stable RPE1-hTERT Cas9 TP53−/− parental and CCNE1-overexpressing clones expressing either a wild type or catalytic-dead FLAG-tagged PKMYT1 sgRNA-resistant ORF. These stable cell lines were transduced with either a LacZ non-targeting sgRNA or PKMYT1 sgRNA #4 and plated at low density to measure their ability to form colonies of >50 cells. After 10 days of growth, the colonies were stained, imaged, and quantified. The results are normalized to the survival of RPE1-hTERT Cas9 TP53−/− CCNE1-overexpressing clones transduced with a non-targeting LacZ control sgRNA and represented as a bar graph in FIG. 5C. Both clones 2 and 21 behave similarly in this study.

FIG. 6 is a chart showing the results of proliferation assays for a panel of CCNE1 wild type and CCNE1-amplified/overexpressing cancer cell lines treated with different doses of compound B. The IC₅₀ values are plotted for each cell line and demonstrate that CCNE1-overexpressing cell lines show enhanced sensitivity to a Myt1 inhibitor compared to CCNE1 WT cell lines.

FIG. 7 is a chart showing the results of proliferation assays for a panel of FBXW7 wild type and FBXW7-mutated cancer cell lines treated with different doses of compound C. The IC50 values are plotted for each cell line and demonstrate that FBXW7-mutated cell lines show enhanced sensitivity to a Myt1 inhibitor compared to FBXW7 WT cell lines.

### DETAILED DESCRIPTION

In general, the invention provides compounds, pharmaceutical compositions containing the same, methods of preparing the compounds, and methods of use. Compounds of the invention may be Myt1 inhibitors. These compounds may be used to inhibit Myt1 in a cell, e.g., a cell in a subject (e.g., a cell overexpressing CCNE1 or having an inactivating mutation in the FBXW7 gene). The subject may be in need of a treatment for a disease or condition, e.g., a disease or condition having a symptom of cell hyperproliferation, e.g., a cancer. The Myt1 inhibitory activity of the compounds disclosed herein is useful for treating a subject in need of a treatment for cancer.

Myt1 is a cell cycle regulating kinase localized predominantly in the endoplasmic reticulum and golgi complex. It is part of the Wee family of kinases that includes Wee1 and Wee1b. It is involved in the negative regulation of the CDK1-Cyclin B complex which promotes the progression of cells from G2-phase into the mitotic phase (M-phase) of the cell cycle. During DNA damage, Myt1 drives the phosphorylation on CDK1 (both Tyr15 and Thr14 of CDK1) which maintains the kinase complex in an inactive state in G2 as part of the G2 checkpoint response along with Wee1 (which mediates only Tyr15 phosphorylation) and prevents entry into mitosis until the damage has been repaired. Additionally, it has been proposed that Myt1 directly interacts with CDK1 complexes in the cytoplasm and prevents their nuclear translocation thus inhibiting cell cycle progression.

Myt1 has been implicated as a potentially important cancer target as it is essential in many cancer cells. Overexpression of Myt1 has been observed in various cancers including hepatocellular carcinoma as well as clear-cell renal-cell carcinoma. Myt1 downregulation has a minor role in unperturbed cells but has a more prominent role in cells exposed to DNA damage. Additionally, cells that exhibit high levels of replication stress in addition to defective G1 checkpoint regulation may be particularly sensitive to loss of Myt1 function, as these cells will be prone to entering mitosis prematurely with compromised genomic material leading to mitotic catastrophe.

Inhibitors of Myt1, a regulator of G2-M transition, may be particularly useful in the treatment of tumors harboring CCNE1-amplification or FBXW7 loss-of-function mutations using a synthetic lethal therapeutic strategy.

Cyclin E1 (encoded by the CCNE1 gene) is involved in the G1 to S phase cell cycle transition. In late G1 phase of the cell cycle, it complexes with cyclin-dependent kinase 2 (CDK2) to promote E2F transcription factor activation and entry into S-phase. Cyclin E1 levels are tightly regulated during normal cell cycles, accumulating at the G1/S transition and being completely degraded by the end of S phase. The cell cycle-dependent proteasomal degradation of Cyclin E1 is mediated by the SCF^{FBW7} ubiquitin ligase complex. Once activated in late G1, the Cyclin E1/CDK2 complex promotes the transition into S phase through phosphorylation and inactivation of RB1 and subsequent release of E2F transcription factors. S phase is promoted by E2F-mediated transcription of numerous genes involved in DNA replication including the pre-replication complex subunits ORC1, CDC6, CDT1, and the MCM helicase factors.

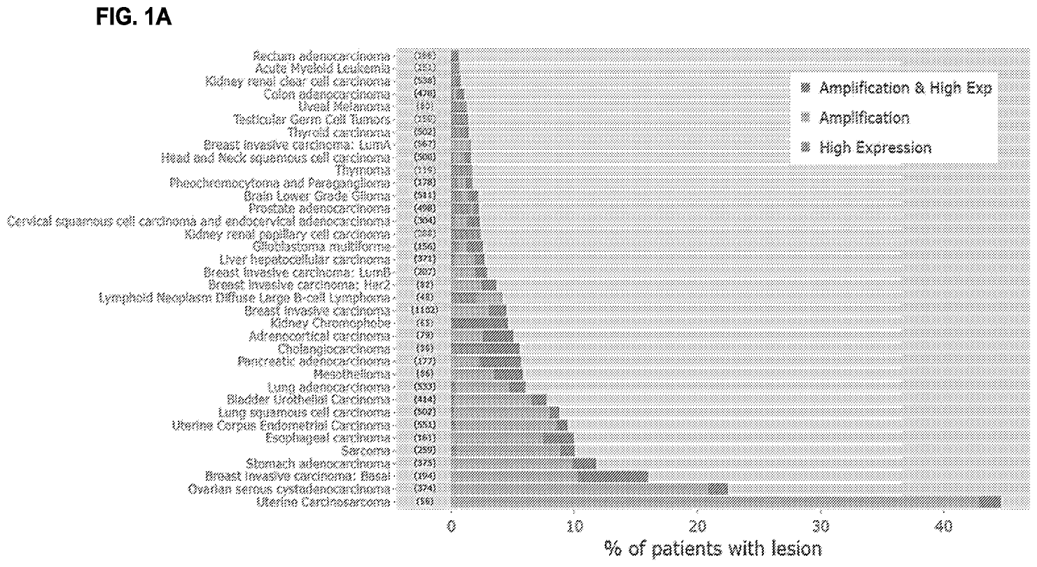

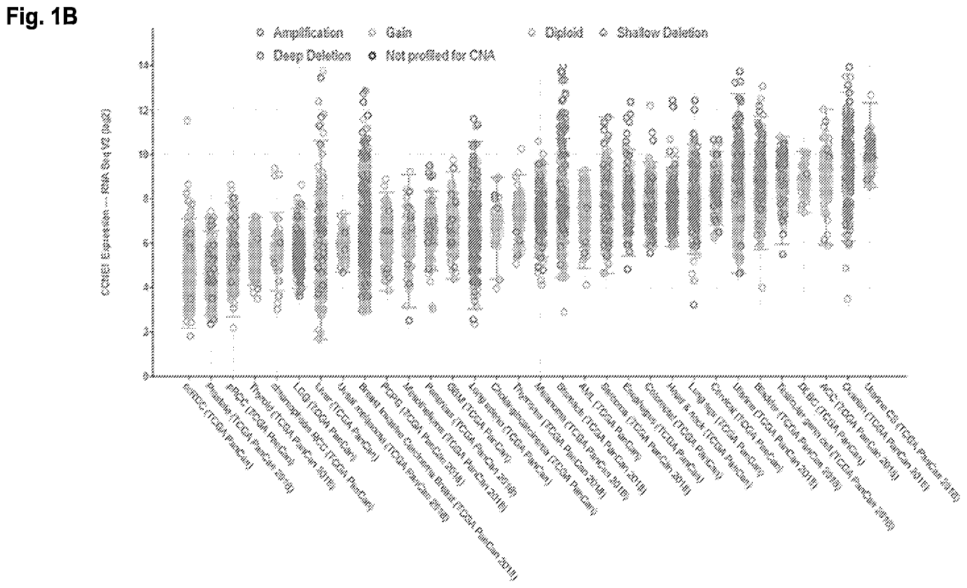

CCNE1 is frequently amplified and/or over-expressed in human cancers (FIG. 1). CCNE1 amplification has been reported in several cancer types including endometrial, ovarian, breast and gastric, ranging in frequency from 5-40%. Importantly, numerous studies have confirmed Cyclin E1 as a driver of tumorigenesis in these indications and CCNE1 amplification is observed in the more aggressive subtypes including uterine carcinosarcoma (UCS; ˜40%), uterine serous carcinoma (USC; ˜25%), high-grade serous ovarian carcinoma (HGSOC; ˜25%), and triple-negative breast cancer (TNBC; ˜8%). Patients with evidence of Cyclin E1 over-expression in tumor biopsies by immunohistochemistry and/or genomic copy number analysis have a lower overall survival compared to patients with normal Cyclin E1 levels. HGSOC patients with Cyclin E1 over-expression have a lower response rate to cisplatin, the current standard of care.

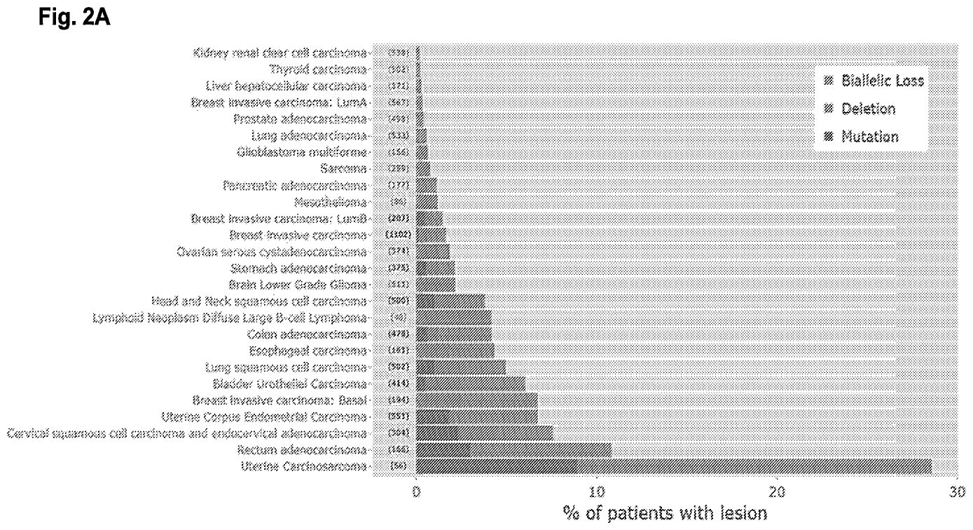

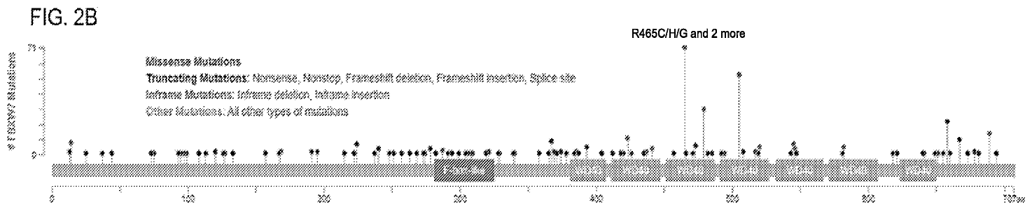

Defective cell cycle-regulated proteolysis of Cyclin E1 by the SCF^{FBW7} ubiquitin ligase complex is another mechanism of CCNE1 over-expression observed in tumors. The F-box protein gene, FBXW7, is frequently mutated in several cancer types including endometrial, colorectal, and gastric, ranging in frequency from 5-35% (FIG. 2). Like CCNE1, FBXW7 driver mutations are observed in the more aggressive subtypes of endometrial cancer including UCS (˜35%) and USC (˜25%). FBXW7 has a diverse spectrum of loss-of-function mutations in cancer including truncating mutations peppered across the gene and missense mutations within the Cyclin E1 recognizing WD40 repeats. FBW7 functions as a homodimer within the SCF complex and many deleterious missense mutations within the WD40 repeats are mostly heterozygous and dominant negative. Remarkably, several recurring hotspot missense mutations are found in the WD40 repeats including R465, R479, and R505—all of which disrupt Cyclin E1 binding and ubiquitylation.

Cyclin E1 over-expression and/or FBXW7 loss-of-function is thought to drive tumorigenesis by inducing genome instability (e.g., increased origin firing, defective nucleotide pools, transcription-replication conflicts, and/or fork instability). Over-expression of Cyclin E1 has been shown to induce replication stress characterized by slowed or stalled replication forks and loss-of-heterozygosity at fragile sites. The primary mechanism by which Cyclin E1 over-expression causes replication stress is increased origin firing in early S-phase followed by depletion of replication factors including nucleotide pools. The decrease in overall replication proteins and nucleotides decreases fork progression and causes stalling and subsequent collapse or reversal.

The compound of the invention may be, e.g., a compound of formula (IA):

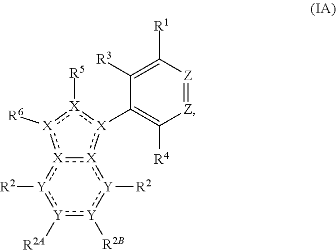

or a pharmaceutically acceptable salt thereof,
wherein

each [symbol] is a single or double bond;

one, two, or three X groups are N, and the remaining X groups are C;

each Y is independently N or C;

each Z is independently N or CH;

R¹ is OH, and R³ is hydrogen, optionally substituted C₁₋₆ alkyl, halogen, or optionally substituted C₃₋₈ cycloalkyl; or R¹ and R³ combine to form —CR⁹═N—NH—;

each R² is independently absent, hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, cyano, —N(R⁷)₂, —OR⁷, —C(O)N(R⁸)₂, —SO₂N(R⁸)₂, —SO₂R^{7A}, or -Q-R^{1B}; and R^{2A} and R^{2B}, together with the atoms to which they are attached, combine to form ring A; or R² and R^{2A}, together with the atoms to which they are attached, combine to form ring A, and R^{2B} is absent or hydrogen;

R⁴ is hydrogen, optionally substituted C₁₋₆ alkyl, halogen, or optionally substituted C₃₋₈ cycloalkyl;

R⁵ is hydrogen, halogen, or —N(R⁷)₂;

R⁶ is —C(O)NH(R⁸), —C(O)R^{7A}, or —SO₂R^{7A};

each R⁷ is independently hydrogen, optionally substituted C₁₋₆ alkyl, optionally substituted C₆₋₁₀ aryl C₁₋₆ alkyl, optionally substituted C₃₋₈ cycloalkyl, optionally substituted C₆₋₁₀ aryl, optionally substituted C₂₋₉ heterocyclyl, optionally substituted C₁₋₉ heteroaryl, optionally substituted C₁₋₉ heteroaryl C₁₋₆ alkyl, or —SO₂R^{7A}; or two R⁷ groups, together with the atom to which both are attached, combine to form an optionally substituted C₂₋₉ heterocyclyl;

each R^{7A} is independently optionally substituted C₁₋₆ alkyl, optionally substituted C₃₋₈ cycloalkyl, or optionally substituted C₆₋₁₀ aryl;

each R^{7B} is independently hydroxyl, optionally substituted C₁₋₆ alkyl, optionally substituted C₆₋₁₀ aryl, optionally substituted C₂₋₉ heterocyclyl, optionally substituted C₁₋₉ heteroaryl, —N(R⁷)₂, —C(O)N(R⁸)₂, —SO₂N(R⁸)₂, —SO₂R^{7A}, or optionally substituted alkoxy;

each R⁸ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or two R⁸, together with the atom to which they are attached, combine to form an optionally substituted C₂₋₉ heterocyclyl;

R⁹ is hydrogen or halogen;

ring A is a 5- or 6-membered carbocyclic ring or a 5- or 6-membered heterocyclic ring, wherein A is optionally substituted with 1, 2, 3, or 4 non-hydrogen R² groups; and

Q is optionally substituted C₁₋₆ alkylene, optionally substituted C₂₋₆ alkenylene, optionally substituted C₂₋₆ alkynylene, optionally substituted C₃₋₈ cycloalkylene, optionally substituted C₃₋₈ cycloalkenylene optionally substituted C₆₋₁₀ arylene, optionally substituted C₂₋₉ heterocyclylene, or optionally substituted C₁₋₉ heteroarylene;

wherein each R² is absent, if attached to Y that is N.

In some embodiments, the compound is a compound of formula (I):

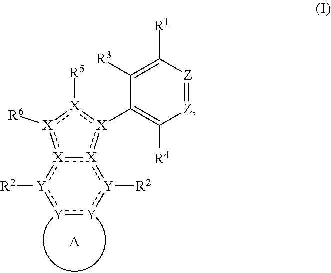

or a pharmaceutically acceptable salt thereof,
where

each [symbol] is a single or double bond;

A is a 5- or 6-membered carbocyclic ring or a 5- or 6-membered heterocyclic ring, where A is optionally substituted with 1, 2, 3, or 4 non-hydrogen R² groups;

one, two, or three X groups are N, and the remaining X groups are C;

each Y is independently N or C;

each Z is independently N or CH;

R¹ is OH, and R³ is hydrogen, optionally substituted C₁₋₆ alkyl, halogen, or optionally substituted C₃₋₈ cycloalkyl; or R¹ and R³ combine to form —CR⁹═N—NH—;

each R² is independently absent, hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, cyano, —N(R⁷)₂, —OR⁷, —C(O)N(R⁸)₂, —SO₂N(R⁸)₂, —SO₂R^{7A}, or -Q-R^{7B};

R⁴ is hydrogen, optionally substituted C₁₋₆ alkyl, halogen, or optionally substituted C₃₋₈ cycloalkyl;

R⁵ is hydrogen, halogen, or —N(R⁷)₂;

R⁶ is —C(O)NH(R⁸), —C(O)R^{7A}, or —SO₂R^{7A};

each R⁷ is independently hydrogen, optionally substituted C₁₋₆ alkyl, optionally substituted C₆₋₁₀ aryl C₁₋₆ alkyl, optionally substituted C₃₋₈ cycloalkyl, optionally substituted C₆₋₁₀ aryl, optionally substituted C₂₋₉ heterocyclyl, optionally substituted C₁₋₉ heteroaryl, optionally substituted C₁₋₉ heteroaryl C₁₋₆ alkyl, or —SO₂R^{7A}; or two R⁷ groups, together with the atom to which both are attached, combine to form an optionally substituted C₂₋₉ heterocyclyl;

each R^{7A} is independently optionally substituted C₁₋₆ alkyl, optionally substituted C₃₋₈ cycloalkyl, or optionally substituted C₆₋₁₀ aryl;

each R^{7B} is independently hydroxyl, optionally substituted C₁₋₆ alkyl, optionally substituted C₆₋₁₀ aryl, optionally substituted C₂₋₉ heterocyclyl, optionally substituted C₁₋₉ heteroaryl, —N(R⁷)₂, —C(O)N(R⁸)₂, —SO₂N(R⁸)₂, —SO₂R^{7A}, or optionally substituted alkoxy;

each R⁸ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or two R⁸, together with the atom to which they are attached, combine to form an optionally substituted C₂₋₉ heterocyclyl;

R⁹ is hydrogen or halogen; and

Q is optionally substituted C₁₋₆ alkylene, optionally substituted C₂₋₆ alkenylene, optionally substituted C₂₋₆ alkynylene, optionally substituted C₃₋₈ cycloalkylene, optionally substituted C₃₋₈ cycloalkenylene optionally substituted C₆₋₁₀ arylene, optionally substituted C₂₋₉ heterocyclylene, or optionally substituted C₁₋₉ heteroarylene;

where each R² is absent, if attached to Y that is N.

The compound of the invention may be, e.g., a compound listed in Table 1 below or a pharmaceutically acceptable salt thereof.

TABLE 1

| | |
|---|---|
| 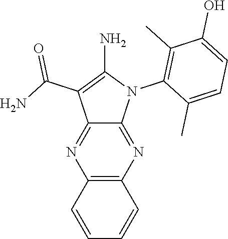 [SMILES: Cc1ccc(O)c(C)c1-n1c(N)c(C(N)=O)c2nc3ccccc3nc21] | 1 |
| 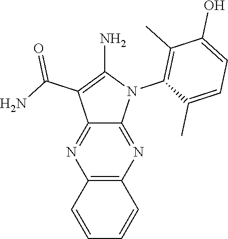 | 2 |
| 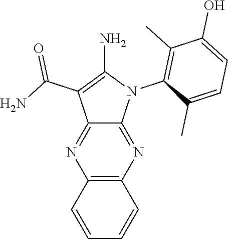 | 3 |
| 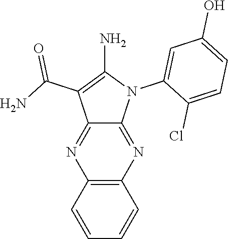 [SMILES: NC(=O)c1c(N)n(-c2cc(O)ccc2Cl)c2nc3ccccc3nc12] | 4 |
| 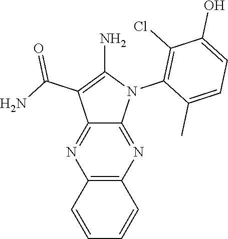 [SMILES: Cc1ccc(O)c(Cl)c1-n1c(N)c(C(N)=O)c2nc3ccccc3nc21] | 5 |
| 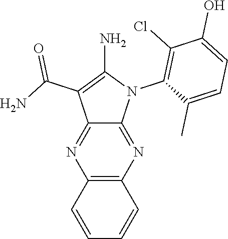 | 6 |
| 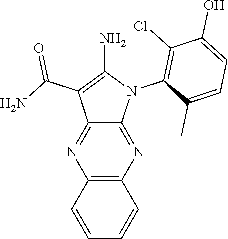 | 7 |
| 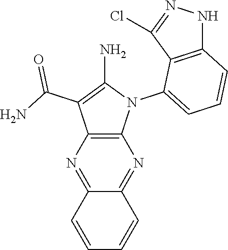 [SMILES: NC(=O)c1c(N)n(-c2cccc3[nH]nc(Cl)c23)c2nc3ccccc3nc12] | 8 |
| 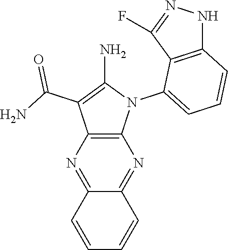 [SMILES: NC(=O)c1c(N)n(-c2cccc3[nH]nc(F)c23)c2nc3ccccc3nc12] | 9 |
| 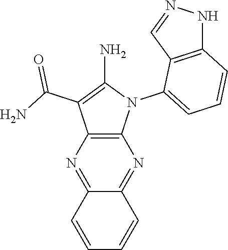 [SMILES: NC(=O)c1c(N)n(-c2cccc3[nH]ncc23)c2nc3ccccc3nc12] | 10 |
| 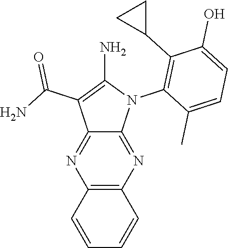 [SMILES: Cc1ccc(O)c(C2CC2)c1-n1c(N)c(C(N)=O)c2nc3ccccc3nc21] | 11 |
| 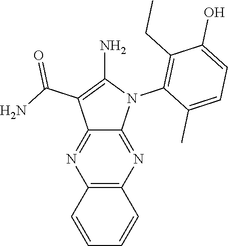 [SMILES: CCc1c(O)ccc(C)c1-n1c(N)c(C(N)=O)c2nc3ccccc3nc21] | 12 |
| 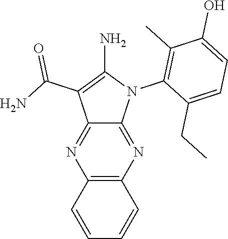 [SMILES: CCc1ccc(O)c(C)c1-n1c(N)c(C(N)=O)c2nc3ccccc3nc21] | 13 |
| 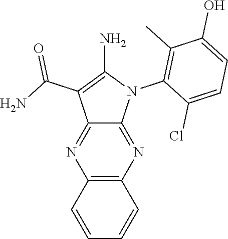 [SMILES: Cc1c(O)ccc(Cl)c1-n1c(N)c(C(N)=O)c2nc3ccccc3nc21] | 14 |
| 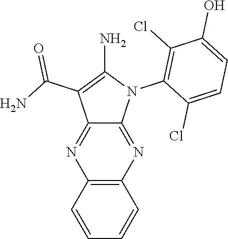 [SMILES: NC(=O)c1c(N)n(-c2c(Cl)ccc(O)c2Cl)c2nc3ccccc3nc12] | 15 |
| 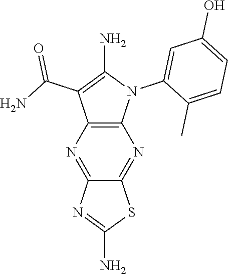 [SMILES: Cc1ccc(O)cc1-n1c(N)c(C(N)=O)c2nc3nc(N)sc3nc21] | 16 |
| 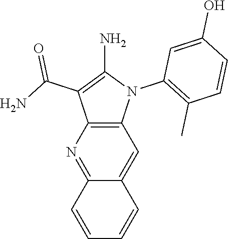 [SMILES: Cc1ccc(O)cc1-n1c(N)c(C(N)=O)c2nc3ccccc3cc21] | 17 |
| 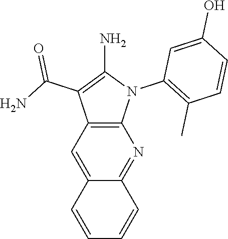 [SMILES: Cc1ccc(O)cc1-n1c(N)c(C(N)=O)c2cc3ccccc3nc21] | 18 |
| 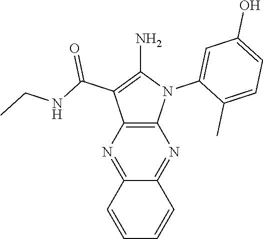 [SMILES: CCNC(=O)c1c(N)n(-c2cc(O)ccc2C)c2nc3ccccc3nc12] | 19 |
| 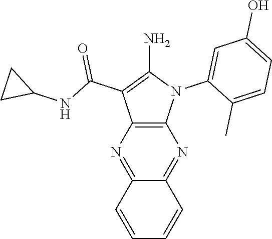 [SMILES: Cc1ccc(O)cc1-n1c(N)c(C(=O)NC2CC2)c2nc3ccccc3nc21] | 20 |
| 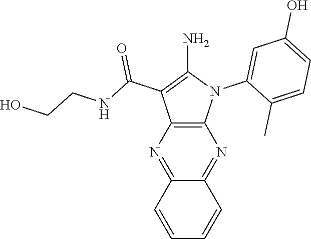 [SMILES: Cc1ccc(O)cc1-n1c(N)c(C(=O)NCCO)c2nc3ccccc3nc21] | 21 |
| 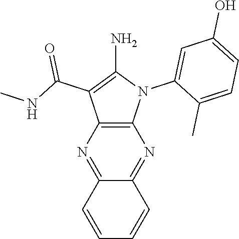 [SMILES: CNC(=O)c1c(N)n(-c2cc(O)ccc2C)c2nc3ccccc3nc12] | 22 |
| 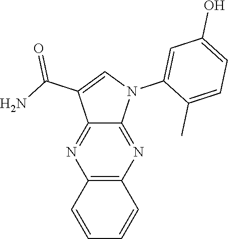 [SMILES: Cc1ccc(O)cc1-n1cc(C(N)=O)c2nc3ccccc3nc21] | 23 |
| 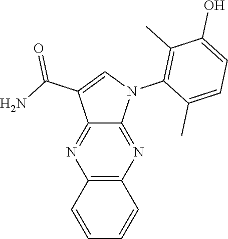 [SMILES: Cc1ccc(O)c(C)c1-n1cc(C(N)=O)c2nc3ccccc3nc21] | 24 |
| 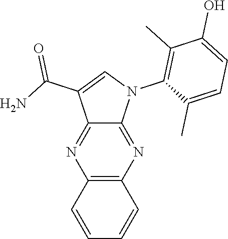 | 25 |
| 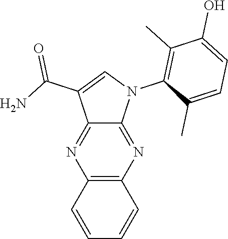 | 26 |
| 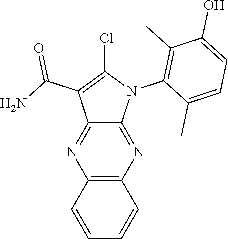 [SMILES: Cc1ccc(O)c(C)c1-n1c(Cl)c(C(N)=O)c2nc3ccccc3nc21] | 27 |
| 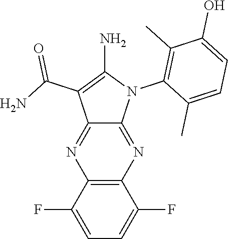 [SMILES: Cc1ccc(O)c(C)c1-n1c(N)c(C(N)=O)c2nc3c(F)ccc(F)c3nc21] | 28 |
| 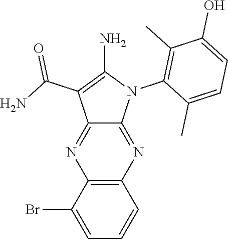 [SMILES: Cc1ccc(O)c(C)c1-n1c(N)c(C(N)=O)c2nc3c(Br)cccc3nc21] | 29 |
| 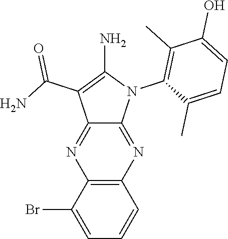 | 30 |
| 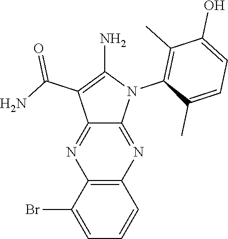 | 31 |
| 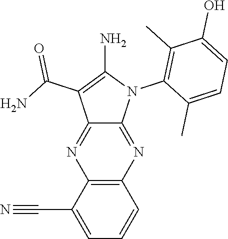 [SMILES: Cc1ccc(O)c(C)c1-n1c(N)c(C(N)=O)c2nc3c(C#N)cccc3nc21] | 32 |
| 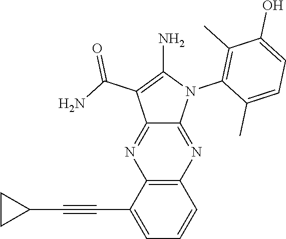 [SMILES: Cc1ccc(O)c(C)c1-n1c(N)c(C(N)=O)c2nc3c(C#CC4CC4)cccc3nc21] | 33 |
| 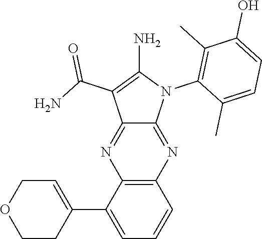 [SMILES: Cc1ccc(O)c(C)c1-n1c(N)c(C(N)=O)c2nc3c(C4=CCOCC4)cccc3nc21] | 34 |
| 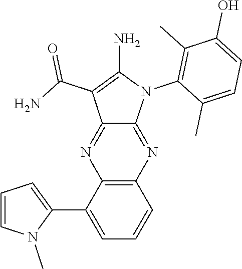 [SMILES: Cc1ccc(O)c(C)c1-n1c(N)c(C(N)=O)c2nc3c(-c4cccn4C)cccc3nc21] | 35 |
| 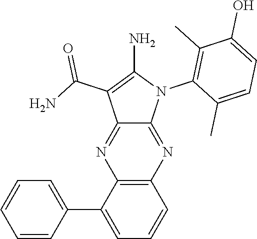 [SMILES: Cc1ccc(O)c(C)c1-n1c(N)c(C(N)=O)c2nc3c(-c4ccccc4)cccc3nc21] | 36 |
| 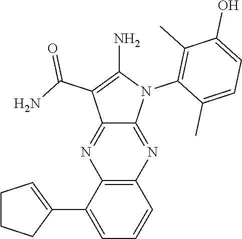 [SMILES: Cc1ccc(O)c(C)c1-n1c(N)c(C(N)=O)c2nc3c(C4=CCCC4)cccc3nc21] | 37 |
| 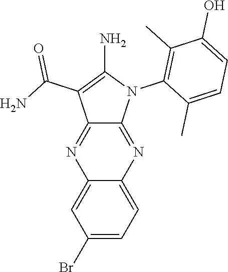 [SMILES: Cc1ccc(O)c(C)c1-n1c(N)c(C(N)=O)c2nc3cc(Br)ccc3nc21] | 38 |
| 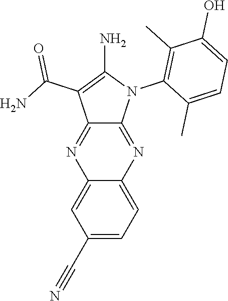 [SMILES: Cc1ccc(O)c(C)c1-n1c(N)c(C(N)=O)c2nc3cc(C#N)ccc3nc21] | 39 |
| 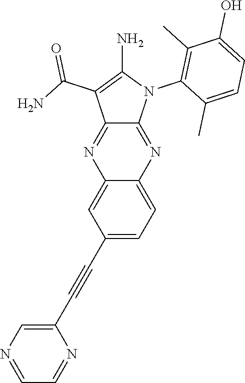 [SMILES: Cc1ccc(O)c(C)c1-n1c(N)c(C(N)=O)c2nc3cc(C#Cc4cnccn4)ccc3nc21] | 40 |
| 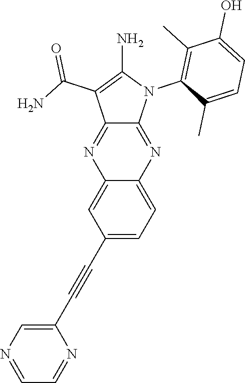 | 41 |
| 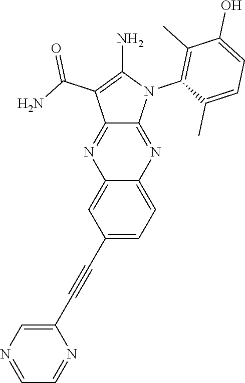 | 42 |
| 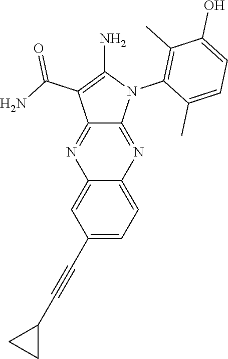 [SMILES: Cc1ccc(O)c(C)c1-n1c(N)c(C(N)=O)c2nc3cc(C#CC4CC4)ccc3nc21] | 43 |
| 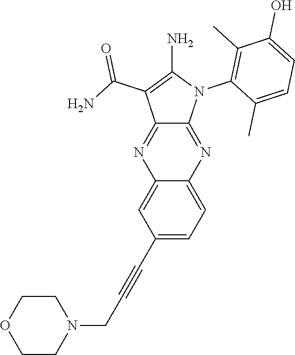 [SMILES: Cc1ccc(O)c(C)c1-n1c(N)c(C(N)=O)c2nc3cc(C#CCN4CCOCC4)ccc3nc21] | 44 |
| 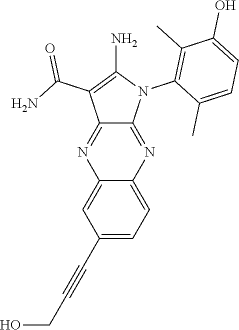 [SMILES: Cc1ccc(O)c(C)c1-n1c(N)c(C(N)=O)c2nc3cc(C#CCO)ccc3nc21] | 45 |
| 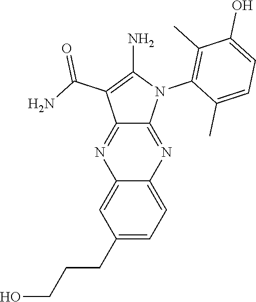 [SMILES: Cc1ccc(O)c(C)c1-n1c(N)c(C(N)=O)c2nc3cc(CCCO)ccc3nc21] | 46 |
| 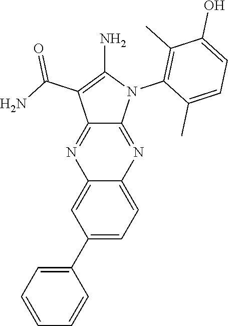 [SMILES: Cc1ccc(O)c(C)c1-n1c(N)c(C(N)=O)c2nc3cc(-c4ccccc4)ccc3nc21] | 47 |
| 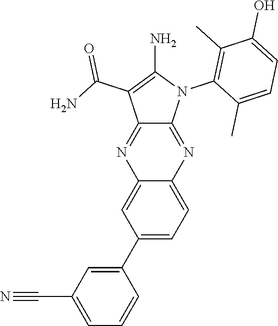 [SMILES: Cc1ccc(O)c(C)c1-n1c(N)c(C(N)=O)c2nc3cc(-c4cccc(C#N)c4)ccc3nc21] | 48 |
| 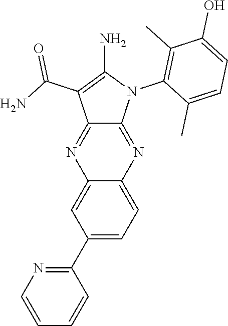 [SMILES: Cc1ccc(O)c(C)c1-n1c(N)c(C(N)=O)c2nc3cc(-c4ccccn4)ccc3nc21] | 49 |
| 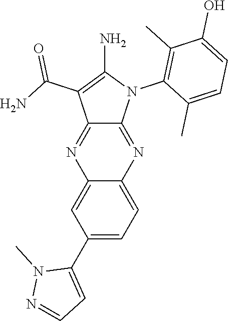 [SMILES: Cc1ccc(O)c(C)c1-n1c(N)c(C(N)=O)c2nc3cc(-c4ccnn4C)ccc3nc21] | 50 |
| 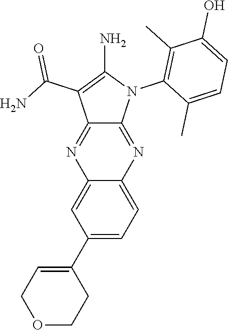 [SMILES: Cc1ccc(O)c(C)c1-n1c(N)c(C(N)=O)c2nc3cc(C4=CCOCC4)ccc3nc21] | 51 |
| 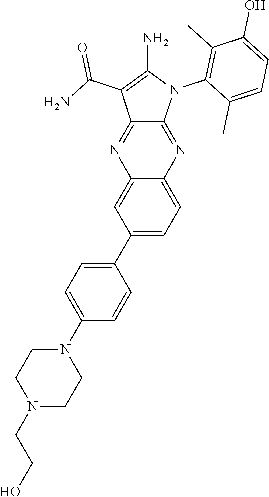 [SMILES: Cc1ccc(O)c(C)c1-n1c(N)c(C(N)=O)c2nc3cc(-c4ccc(N5CCN(CCO)CC5)cc4)ccc3nc21] | 52 |
| 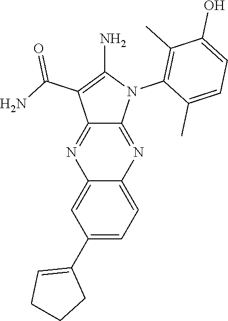 [SMILES: Cc1ccc(O)c(C)c1-n1c(N)c(C(N)=O)c2nc3cc(C4=CCCC4)ccc3nc21] | 53 |
| 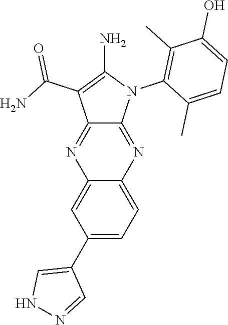 [SMILES: Cc1ccc(O)c(C)c1-n1c(N)c(C(N)=O)c2nc3cc(-c4cn[nH]c4)ccc3nc21] | 54 |
| 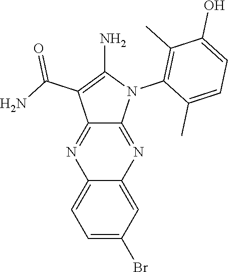 [SMILES: Cc1ccc(O)c(C)c1-n1c(N)c(C(N)=O)c2nc3ccc(Br)cc3nc21] | 55 |
| 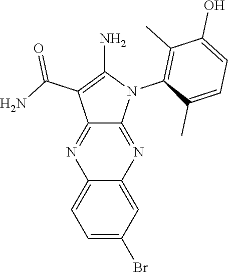 | 56 |
| 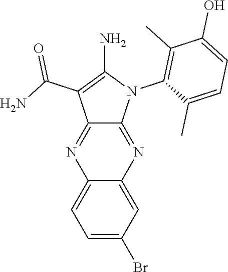 | 57 |
| 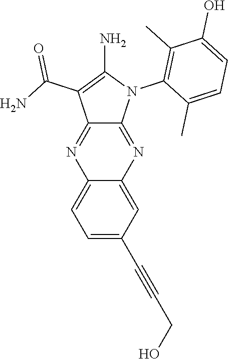 [SMILES: Cc1ccc(O)c(C)c1-n1c(N)c(C(N)=O)c2nc3ccc(C#CCO)cc3nc21] | 58 |
| 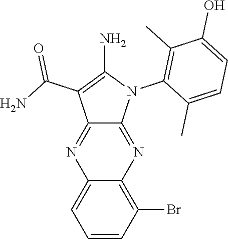 [SMILES: Cc1ccc(O)c(C)c1-n1c(N)c(C(N)=O)c2nc3cccc(Br)c3nc21] | 59 |
| 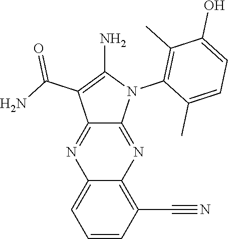 [SMILES: Cc1ccc(O)c(C)c1-n1c(N)c(C(N)=O)c2nc3cccc(C#N)c3nc21] | 60 |
| 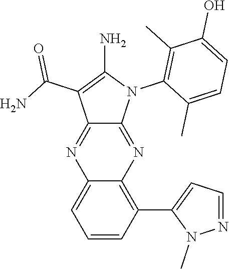 [SMILES: Cc1ccc(O)c(C)c1-n1c(N)c(C(N)=O)c2nc3cccc(-c4ccnn4C)c3nc21] | 61 |
| 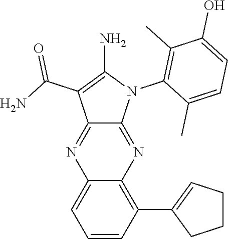 [SMILES: Cc1ccc(O)c(C)c1-n1c(N)c(C(N)=O)c2nc3cccc(C4=CCCC4)c3nc21] | 62 |
| (one of the structures below) 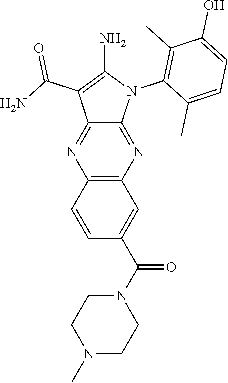 [SMILES: Cc1ccc(O)c(C)c1-n1c(N)c(C(N)=O)c2nc3ccc(C(=O)N4CCN(C)CC4)cc3nc21] | 63 |
| 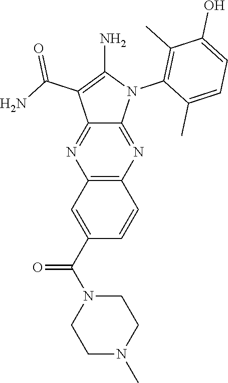 [SMILES: Cc1ccc(O)c(C)c1-n1c(N)c(C(N)=O)c2nc3cc(C(=O)N4CCN(C)CC4)ccc3nc21] | |
| (one of the structures below) 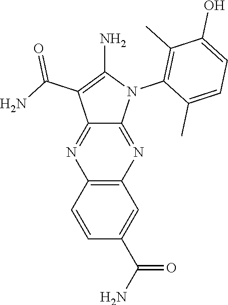 [SMILES: Cc1ccc(O)c(C)c1-n1c(N)c(C(N)=O)c2nc3ccc(C(N)=O)cc3nc21] | 64 |
| 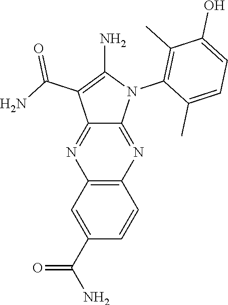 [SMILES: Cc1ccc(O)c(C)c1-n1c(N)c(C(N)=O)c2nc3cc(C(N)=O)ccc3nc21] | |
| (one of the structures below) 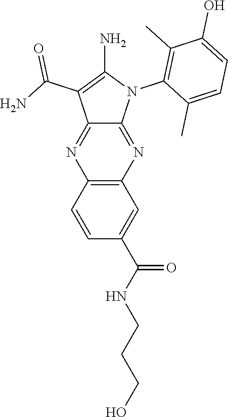 [SMILES: Cc1ccc(O)c(C)c1-n1c(N)c(C(N)=O)c2nc3ccc(C(=O)NCCCO)cc3nc21] | 65 |
| 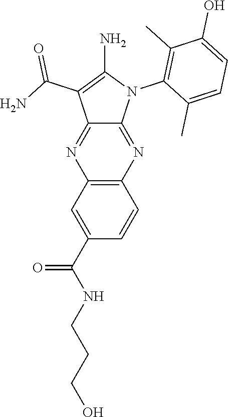 [SMILES: Cc1ccc(O)c(C)c1-n1c(N)c(C(N)=O)c2nc3cc(C(=O)NCCCO)ccc3nc21] | |
| (one of the structures below) 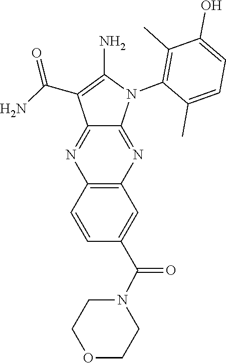 [SMILES: Cc1ccc(O)c(C)c1-n1c(N)c(C(N)=O)c2nc3ccc(C(=O)N4CCOCC4)cc3nc21] | 66 |
| 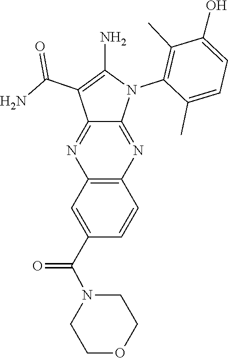 [SMILES: Cc1ccc(O)c(C)c1-n1c(N)c(C(N)=O)c2nc3cc(C(=O)N4CCOCC4)ccc3nc21] | |
| (one of the structures below) 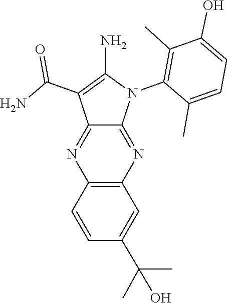 [SMILES: Cc1ccc(O)c(C)c1-n1c(N)c(C(N)=O)c2nc3ccc(C(C)(C)O)cc3nc21] | 67 |
| 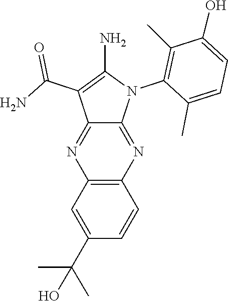 [SMILES: Cc1ccc(O)c(C)c1-n1c(N)c(C(N)=O)c2nc3cc(C(C)(C)O)ccc3nc21] | |
| (one of the structures below) 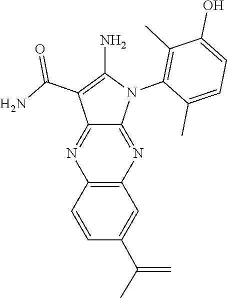 [SMILES: C=C(C)c1ccc2nc3c(C(N)=O)c(N)n(-c4c(C)ccc(O)c4C)c3nc2c1] | 68 |
| 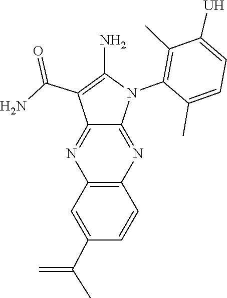 | |
| (one of the structures below) 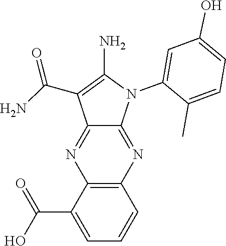 [SMILES: Cc1ccc(O)cc1-n1c(N)c(C(N)=O)c2nc3c(C(=O)O)cccc3nc21] | 69 |
| 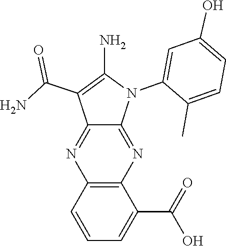 [SMILES: Cc1ccc(O)cc1-n1c(N)c(C(N)=O)c2nc3cccc(C(=O)O)c3nc21] | |
| (one of the structures below) 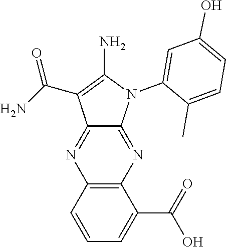 [SMILES: Cc1ccc(O)cc1-n1c(N)c(C(N)=O)c2nc3cccc(C(=O)O)c3nc21] | 70 |
| 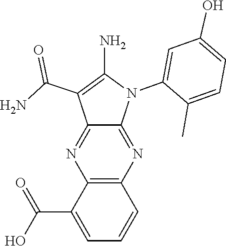 [SMILES: Cc1ccc(O)cc1-n1c(N)c(C(N)=O)c2nc3c(C(=O)O)cccc3nc21] | |
| (one of the structures below) 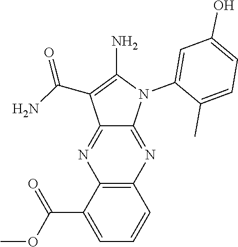 [SMILES: COC(=O)c1cccc2nc3c(nc12)c(C(N)=O)c(N)n3-c1cc(O)ccc1C] | 71 |
| 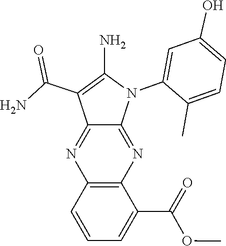 [SMILES: COC(=O)c1cccc2nc3c(C(N)=O)c(N)n(-c4cc(O)ccc4C)c3nc12] | |
| (one of the structures below) 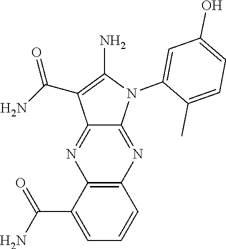 [SMILES: Cc1ccc(O)cc1-n1c(N)c(C(N)=O)c2nc3c(C(N)=O)cccc3nc21] | 72 |
| 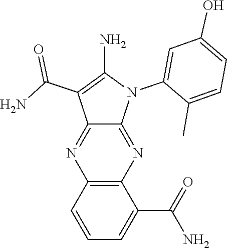 [SMILES: Cc1ccc(O)cc1-n1c(N)c(C(N)=O)c2nc3cccc(C(N)=O)c3nc21] | |
| (one of the structures below) 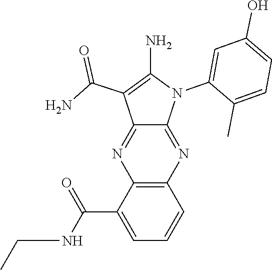 [SMILES: CCNC(=O)c1cccc2nc3c(nc12)c(C(N)=O)c(N)n3-c1cc(O)ccc1C] | 73 |
| 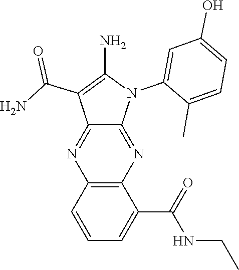 [SMILES: CCNC(=O)c1cccc2nc3c(C(N)=O)c(N)n(-c4cc(O)ccc4C)c3nc12] | |
| (one of the structures below) 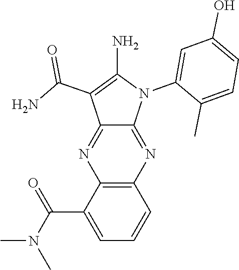 [SMILES: Cc1ccc(O)cc1-n1c(N)c(C(N)=O)c2nc3c(C(=O)N(C)C)cccc3nc21] | 74 |
| 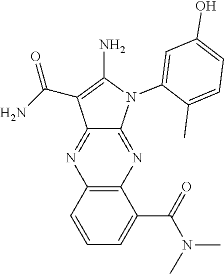 [SMILES: Cc1ccc(O)cc1-n1c(N)c(C(N)=O)c2nc3cccc(C(=O)N(C)C)c3nc21] | |
| (one of the structures below) 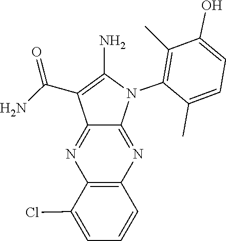 [SMILES: Cc1ccc(O)c(C)c1-n1c(N)c(C(N)=O)c2nc3c(Cl)cccc3nc21] | 75 |
| 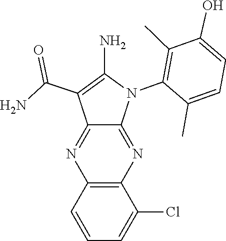 [SMILES: Cc1ccc(O)c(C)c1-n1c(N)c(C(N)=O)c2nc3cccc(Cl)c3nc21] | |
| (one of the structures below) 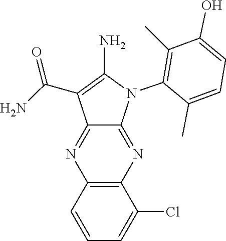 [SMILES: Cc1ccc(O)c(C)c1-n1c(N)c(C(N)=O)c2nc3cccc(Cl)c3nc21] | 76 |
| 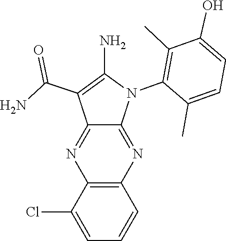 [SMILES: Cc1ccc(O)c(C)c1-n1c(N)c(C(N)=O)c2nc3c(Cl)cccc3nc21] | |
| (one of the structures below) 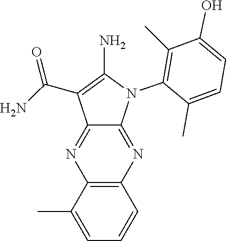 [SMILES: Cc1ccc(O)c(C)c1-n1c(N)c(C(N)=O)c2nc3c(C)cccc3nc21] | 77 |
| 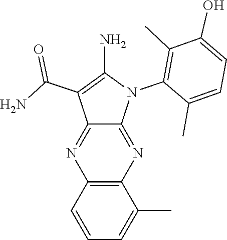 [SMILES: Cc1ccc(O)c(C)c1-n1c(N)c(C(N)=O)c2nc3cccc(C)c3nc21] | |
| (one of the structures below) 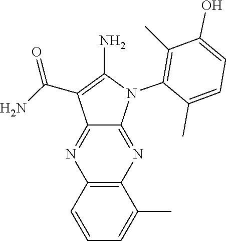 [SMILES: Cc1ccc(O)c(C)c1-n1c(N)c(C(N)=O)c2nc3cccc(C)c3nc21] | 78 |
| 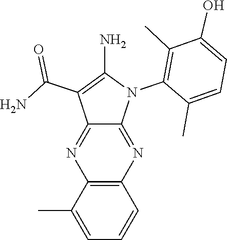 [SMILES: Cc1ccc(O)c(C)c1-n1c(N)c(C(N)=O)c2nc3c(C)cccc3nc21] | |
| (one of the structures below) 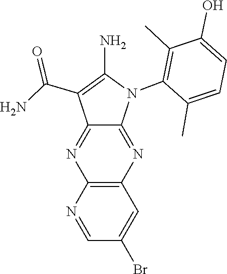 [SMILES: Cc1ccc(O)c(C)c1-n1c(N)c(C(N)=O)c2nc3ncc(Br)cc3nc21] | 79 |
| 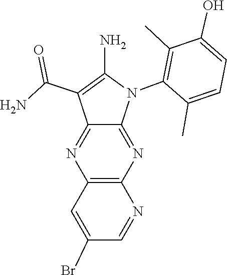 [SMILES: Cc1ccc(O)c(C)c1-n1c(N)c(C(N)=O)c2nc3cc(Br)cnc3nc21] | |
| (one of the structures below) 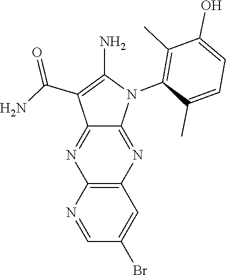 | 80 |
| 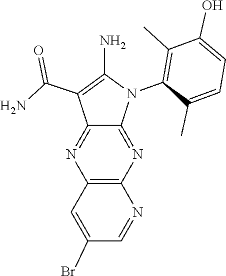 | |
| (one of the structures below) 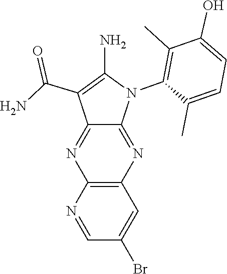 | 81 |
| 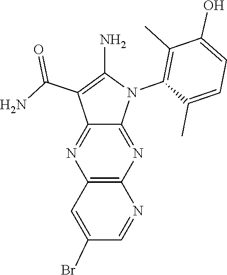 | |
| (one of the structures below) 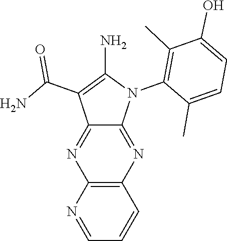 [SMILES: Cc1ccc(O)c(C)c1-n1c(N)c(C(N)=O)c2nc3ncccc3nc21] | 82 |
| 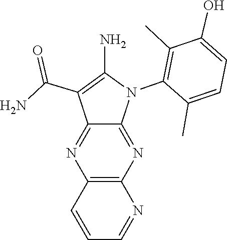 [SMILES: Cc1ccc(O)c(C)c1-n1c(N)c(C(N)=O)c2nc3cccnc3nc21] | |
| (one of the structures below) 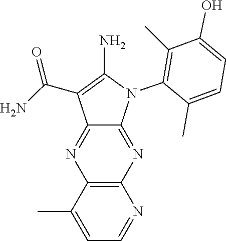 [SMILES: Cc1ccc(O)c(C)c1-n1c(N)c(C(N)=O)c2nc3c(C)ccnc3nc21] | 83 |
| 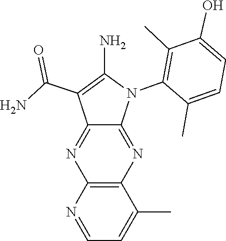 [SMILES: Cc1ccc(O)c(C)c1-n1c(N)c(C(N)=O)c2nc3nccc(C)c3nc21] | |
| 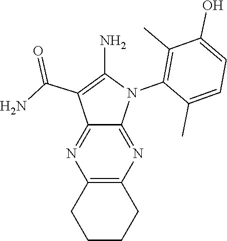 [SMILES: Cc1ccc(O)c(C)c1-n1c(N)c(C(N)=O)c2nc3c(nc21)CCCC3] | 84 |
| 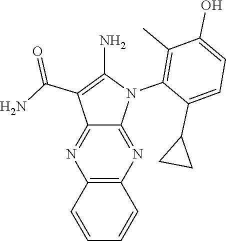 [SMILES: Cc1c(O)ccc(C2CC2)c1-n1c(N)c(C(N)=O)c2nc3ccccc3nc21] | 85 |
| 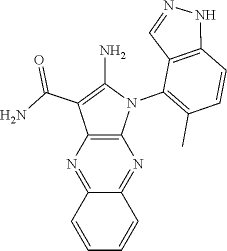 [SMILES: Cc1ccc2[nH]ncc2c1-n1c(N)c(C(N)=O)c2nc3ccccc3nc21] | 86 |
| 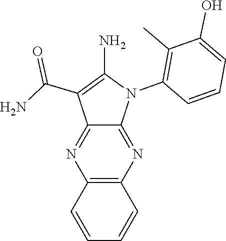 [SMILES: Cc1c(O)cccc1-n1c(N)c(C(N)=O)c2nc3ccccc3nc21] | 87 |
| 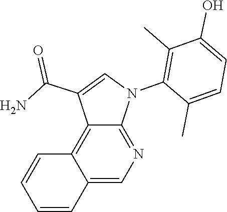 [SMILES: Cc1ccc(O)c(C)c1-n1cc(C(N)=O)c2c3ccccc3cnc21] | 88 |
| 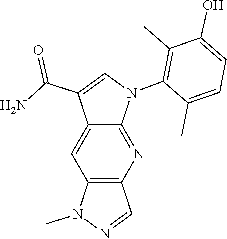 [SMILES: Cc1ccc(O)c(C)c1-n1cc(C(N)=O)c2cc3c(cnn3C)nc21] | 89 |

The invention includes (where possible) individual diastereomers, enantiomers, epimers, and atropisomers of the compounds disclosed herein, and mixtures of diastereomers and/or enantiomers thereof including racemic mixtures. Although the specific stereochemistries disclosed herein are preferred, other stereoisomers, including diastereomers, enantiomers, epimers, atropisomers, and mixtures of these may also have utility in treating Myt1-mediated diseases. Inactive or less active diastereoisomers and enantiomers may be useful, e.g., for scientific studies relating to the receptor and the mechanism of activation.

It is understood that certain molecules can exist in multiple tautomeric forms. This invention includes all tautomers even though only one tautomer may be indicated in the examples.

The invention also includes pharmaceutically acceptable salts of the compounds, and pharmaceutical compositions comprising the compounds and a pharmaceutically acceptable carrier. The compounds are especially useful, e.g., in certain kinds of cancer and for slowing the progression of cancer once it has developed in a patient.

The compounds disclosed herein may be used in pharmaceutical compositions comprising (a) the compound(s) or pharmaceutically acceptable salts thereof, and (b) a pharmaceutically acceptable carrier. The compounds may be used in pharmaceutical compositions that include one or more other active pharmaceutical ingredients. The compounds may also be used in pharmaceutical compositions in which the compound disclosed herein or a pharmaceutically acceptable salt thereof is the only active ingredient.

Optical Isomers—Diastereomers—Geometric Isomers—Tautomers

Compounds disclosed herein may contain, e.g., one or more stereogenic centers and can occur as racemates, racemic mixtures, single enantiomers, individual diastereomers, and mixtures of diastereomers and/or enantiomers. The invention includes all such isomeric forms of the compounds disclosed herein. It is intended that all possible stereoisomers (e.g., enantiomers and/or diastereomers) in mixtures and as pure or partially purified compounds are included within the scope of this invention (i.e., all possible combinations of the stereogenic centers as pure compounds or in mixtures).

Some of the compounds described herein may contain bonds with hindered rotation such that two separate rotomers, or atropisomers, may be separated and found to have different biological activity which may be advantageous. It is intended that all of the possible atropisomers are included within the scope of this invention.

Some of the compounds described herein may contain olefinic double bonds, and unless specified otherwise, are meant to include both E and Z geometric isomers.

Some of the compounds described herein may exist with different points of attachment of hydrogen, referred to as tautomers. An example is a ketone and its enol form, known as keto-enol tautomers. The individual tautomers as well as mixtures thereof are encompassed by the invention.

Compounds disclosed herein having one or more asymmetric centers may be separated into diastereoisomers, enantiomers, and the like by methods well known in the art.

Alternatively, enantiomers and other compounds with chiral centers may be synthesized by stereospecific synthesis using optically pure starting materials and/or reagents of known configuration.

Metabolites—Prodrugs

The invention includes therapeutically active metabolites, where the metabolites themselves fall within the scope of the claims. The invention also includes prodrugs, which are compounds that are converted to the claimed compounds as they are being administered to a patient or after they have been administered to a patient. The claimed chemical structures of this application in some cases may themselves be prodrugs.

Isotopically Enriched Derivatives

The invention includes molecules which have been isotopically enriched at one or more position within the molecule. Thus, compounds enriched for deuterium fall within the scope of the claims.

### Methods of Preparing a Compound of the Invention

Compounds of the present invention may be prepared using reactions and techniques known in the art and those described herein. One of skill in the art will appreciate that methods of preparing compounds of the invention described herein are non-limiting and that steps within the methods may be interchangeable without affecting the structure of the end product.

### Method A

Compounds of the present invention may be prepared as shown in Scheme A and described herein. One chloro of a 2,3-dichloropyrazine can be substituted by malononitrile under S_{N}Ar or palladium-mediated conditions. The remaining chloro can be substituted with an aromatic amine under S_{N}Ar or palladium-mediated conditions to afford an aminopyrrole. Depending on the nature of the arylamine, a protecting group may be required to be in place prior to this reaction. Hydrolysis of the nitrile can be done under acidic or basic conditions to give compounds of the present invention. In the case where the arylamine group bears a protecting group, a deprotection step(s) may be required using acid, base and/or fluoride to give compounds of the present invention. Fused pyrrolopyrazine core may bear substituent(s) which can be substituted and/or derivatized at any step in the synthesis including at the end. In some cases, a single or major regioisomer is obtained. In other cases, a mixture of regioisomers is separated. In some instances, the regiochemistry is known, in some other instances, a single regioisomer is depicted with two possible configurations. Depending on the nature of the arylamine, an atropisomeric mixture may be obtained. In such cases it may be necessary to isolate the atropisomer of interest to give compounds of the present invention. Alternatively, an atropisomerically enriched (e.g., pure) intermediate can be isolated and may be derivatized further to give compounds of the present invention.

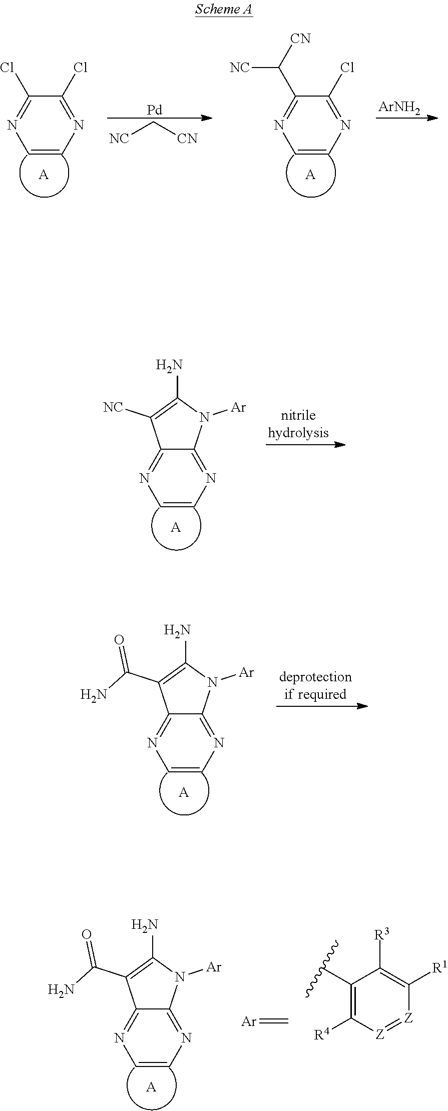

### Method B

Compounds of the present invention may be prepared as shown in Scheme B and described herein. One chloro of a 2,3-dichloropyrazine can be substituted by an aromatic amine under S_{N}Ar or palladium-mediated conditions. The remaining chloro can be substituted with malononitrile under S_{N}Ar or palladium-mediated conditions to afford an aminopyrrole. Depending on the nature of the arylamine, a protecting group may be required to be in place prior to this reaction. Hydrolysis of the nitrile can be done under acidic or basic conditions to give compounds of the present invention. In the case where the arylamine group bears a protecting group, a deprotection step(s) may be required using acid, base and/or fluoride to give compounds of the present invention. Fused pyrrolopyrazine core may bear substituent(s) which can be substituted and/or derivatized at any step in the synthesis including at the end. In some cases, a single or major regioisomer is obtained. In other cases, a mixture of regioisomers is separated. In some instances, the regiochemistry is known, in some other instances, a single regioisomer is depicted with two possible configurations. Depending on the nature of the arylamine, an atropisomeric mixture may be obtained. In such cases it may be necessary to isolate the atropisomer of interest to give compounds of the present invention. Alternatively, an atropisomerically enriched (e.g., pure) intermediate can be isolated and may be derivatized further to give compounds of the present invention.

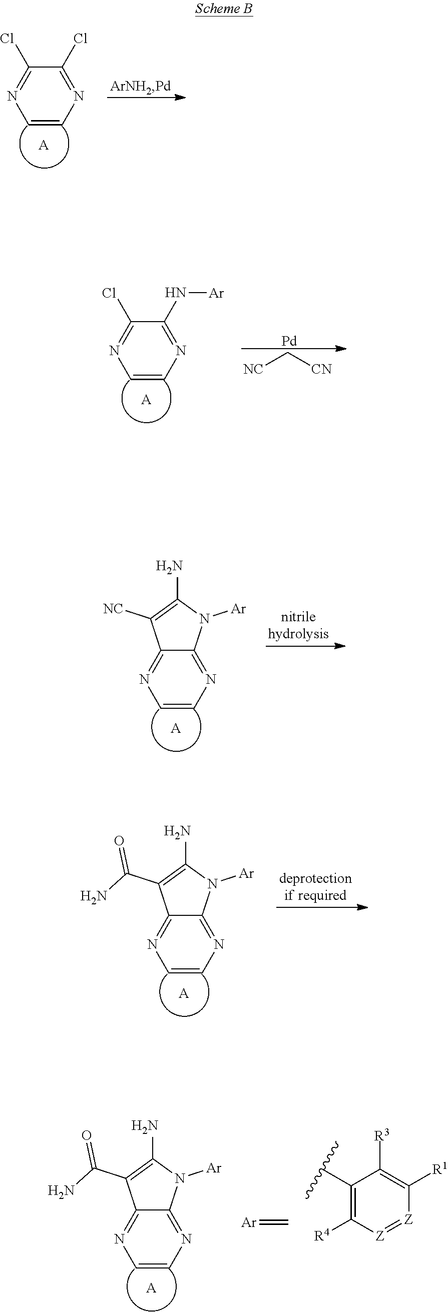

### Method C

Compounds of the present invention may be prepared as shown in Scheme C and described herein. Commercially available 3,5-dibromo-6-chloro-pyrazin-2-amine may be condensed with a substituted isothiocyanate to provide a fused thiazolopyrazine. The bromo can be substituted with the anion of malononitrile under palladium catalyzed conditions and the chloro can be substituted with an arylamine to afford the fused pyrrolothiazolopyrazine described herein. Hydrolysis of the nitrile can be done under acidic or basic conditions to give compounds of the present invention. In the case where the arylamine group bears a protecting group, a deprotection step(s) may be required using acid, base and/or fluoride to give compounds of the present invention. The R substituent may be removed or modified at any step in the synthesis including at the end. Depending on the nature of the arylamine, an atropisomeric mixture may be obtained. In such cases it may be necessary to isolate the atropisomer of interest to give compounds of the present invention. Alternatively, an atropisomerically enriched (e.g., pure) intermediate can be isolated and may be derivatized further to give compounds of the present invention.

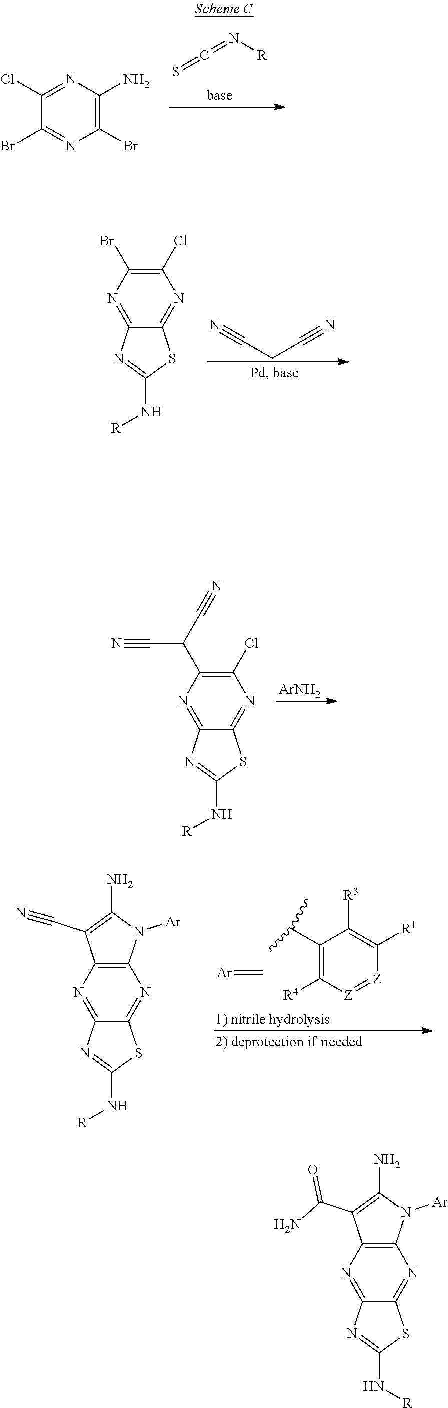

### Method D

Compounds of the present invention may be prepared as shown in Scheme D and described herein. The Bromo of commercially available 3-bromo-2-chloropyridyl ring system can be selectively substituted with an arylamine under palladium catalyzed conditions. The remaining chloro can be substituted with the anion of malononitrile under S_{N}Ar conditions, which cyclizes to a substituted pyrrolopyridyl tricyclic ring system. In the case where the arylamine group bears a protecting group, a deprotection step(s) may be required using acid, base, and/or fluoride to give compounds of the present invention. This deprotection step may be realized before hydrolysis of the nitrile which can be done under acidic or basic conditions to give compounds of the present invention. Fused pyrrolopyridyl tricyclic core may bear substituent(s) which can be substituted and/or derivatized at any step in the synthesis including at the end. Depending on the nature of the arylamine, an atropisomeric mixture may be obtained. In such cases it may be necessary to isolate the atropisomer of interest to give compounds of the present invention. Alternatively, an atropisomerically enriched (e.g., pure) intermediate can be isolated and may be derivatized further to give compounds of the present invention.

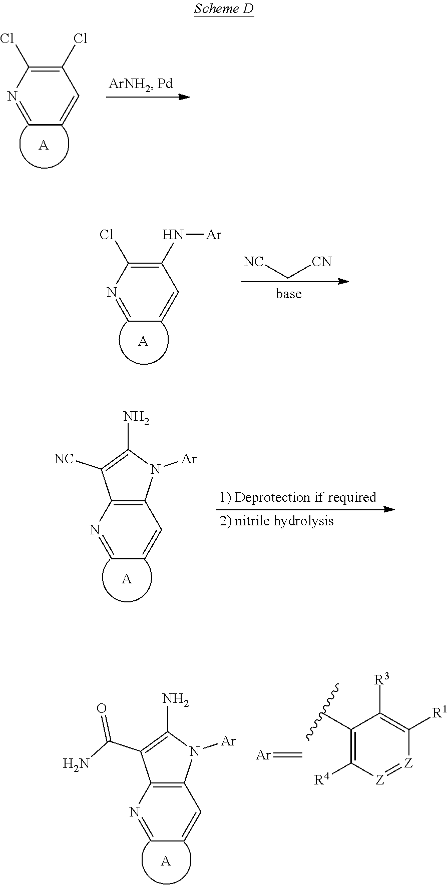

### Method E

Compounds of the present invention may be prepared as shown in Scheme E and described herein. The 2-halogen atom of 2,3-dihalogenopyrido bicycle can be selectively substituted with an arylamine under SNAr or palladium catalyzed conditions. The remaining halogen can be substituted with malononitrile under palladium-catalyzed conditions to provide the aminopyrrole. In the case where the arylamine group bears a protecting group, a deprotection step(s) may be required using acid, base and/or fluoride to give compounds of the present invention. This deprotection step may be realized before hydrolysis of the nitrile which can be done under acidic or basic conditions to give compounds of the present invention. Fused pyrrolopyridyl tricyclic core may bear substituent(s) which can be substituted and/or derivatized at any step in the synthesis including at the end. Depending on the nature of the arylamine, an atropisomeric mixture may be obtained. In such cases it may be necessary to isolate the atropisomer of interest to give compounds of the present invention. Alternatively, an atropisomerically enriched (e.g., pure) intermediate can be isolated and may be derivatized further to give compounds of the present invention.

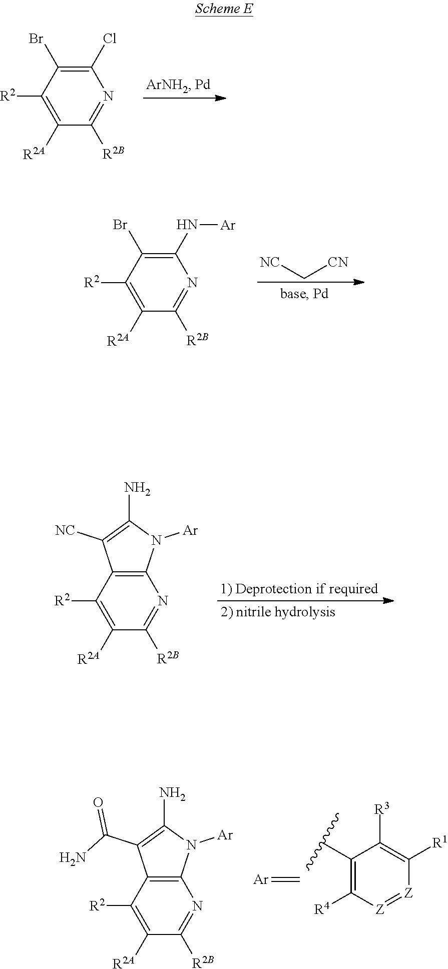

### Method F

Compounds of the present invention may be prepared as shown in Scheme F and described herein. Using a procedure similar to method B, one chloro of a fused 2,3-dichloropyrazine can be substituted by an aromatic amine under S_{N}Ar or palladium-mediated conditions. The remaining chloro can be substituted with a 2-cyanoacetamide under S_{N}Ar or palladium-mediated conditions to afford an aminopyrrole. Depending on the nature of the arylamine, a protecting group may be required to be in place prior to this reaction. In the case where the arylamine group bears a protecting group, a deprotection step(s) may be required using acid, base and/or fluoride to give compounds of the present invention. Fused pyrrolopyrazine core may bear substituent(s) which can be substituted and/or derivatized at any step in the synthesis including at the end. Depending on the nature of the arylamine, an atropisomeric mixture may be obtained. In such cases it may be necessary to isolate the atropisomer of interest to give compounds of the present invention. Alternatively, an atropisomerically enriched (e.g., pure) intermediate can be isolated and may be derivatized further to give compounds of the present invention.

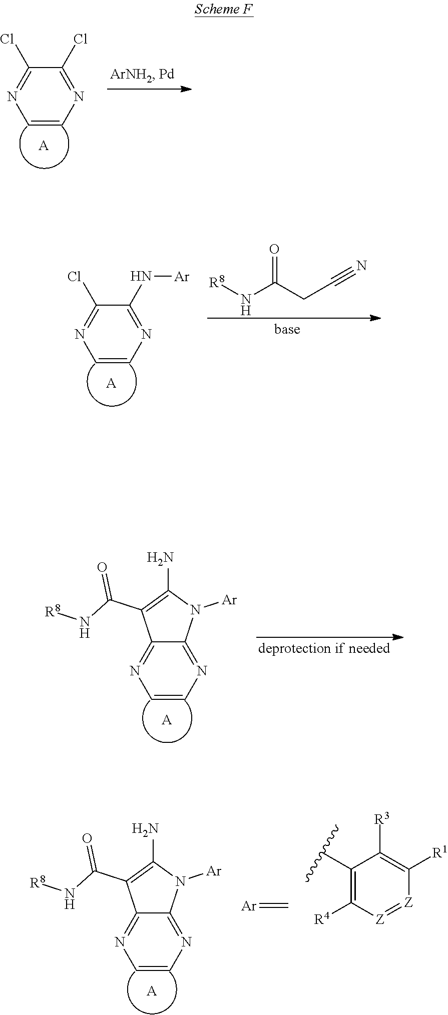

### Method G

Compounds of the present invention may be prepared as shown in Scheme G and described herein. The amino of an aminopyrrole described herein can be substituted by a proton or a chlorine under diazotization conditions. The nitrile can be hydrolyzed to the carboxamide under acidic or basic conditions to give compound of the present invention. Alternatively, the nitrile may be hydrolyzed prior to substitution of the amino group. In the case where the arylamine group bears a protecting group, a deprotection step(s) may be required using acid, base and/or fluoride to give compounds of the present invention. A similar protocol may be applied to the other fused bicycles from the methods to substitute the amino group by a proton or chlorine. Depending on the nature of the N-aryl group, an atropisomeric mixture may be obtained. In such cases it may be necessary to isolate the atropisomer of interest to give compounds of the present invention. Alternatively, an atropisomerically enriched (e.g., pure) intermediate can be isolated and may be derivatized further to give compounds of the present invention.

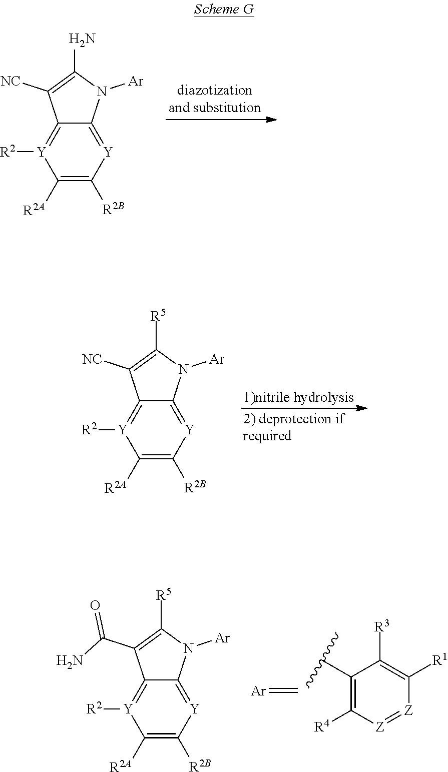

### Method H

Compounds of the present invention may be prepared as shown in Scheme H and described herein. The halogen or triflate of a fused tricyclic system constructed via one of the methods described herein and bearing a halogen or triflate substituent at any of the 4 positions on the fused aryl ring may be substituted with a cyano group. In the case where the arylamine group bears a protecting group, a deprotection step(s) may be required using acid, base and/or fluoride to give compounds of the present invention. Depending on the nature of the arylamine, an atropisomeric mixture may be obtained. In such cases it may be necessary to isolate the atropisomer of interest to give compounds of the present invention. Alternatively, an atropisomerically enriched (e.g., pure) intermediate can be isolated and may be derivatized further to give compounds of the present invention.

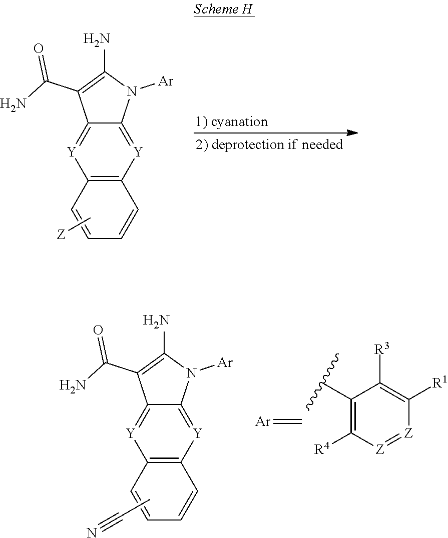

### Method I

Compounds of the present invention may be prepared as shown in Scheme I and described herein. The halogen or triflate of a fused tricyclic system constructed via one of the methods described herein and bearing a halogen or triflate at any of the 4 positions on the fused aryl ring may be substituted with an alkynyl group by a Sonogashira-type metal mediated coupling reaction with a substituted alkyne. In the case where the arylamine group bears a protecting group, a deprotection step(s) may be required using acid, base and/or fluoride to give compounds of the present invention. The alkyne may optionally be reduced to produce the corresponding substituted alkyl. In other cases, the alkyne substituent may bear a protecting group which may be removed concomitantly or in a separate step. Depending on the nature of the arylamine, an atropisomeric mixture may be obtained. In such cases it may be necessary to isolate the atropisomer of interest to give compounds of the present invention. Alternatively, an atropisomerically enriched (e.g., pure) intermediate can be isolated and may be derivatized further to give compounds of the present invention.

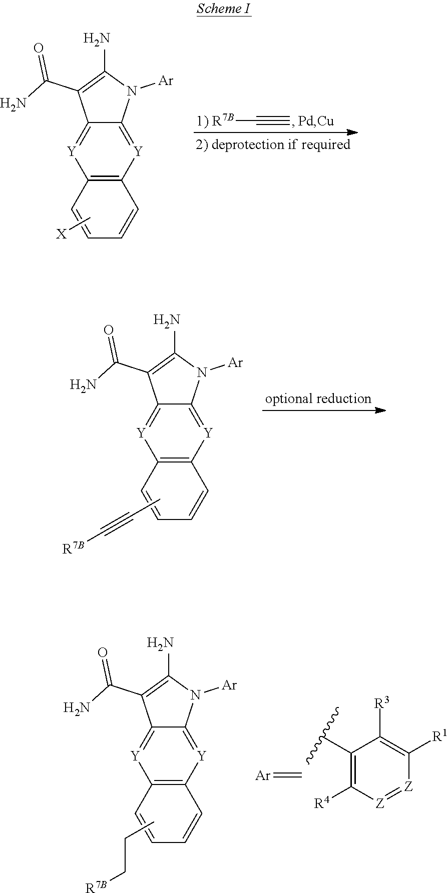

### Method J

Compounds of the present invention may be prepared as shown in Scheme J and described herein. The halogen or triflate of a fused tricyclic system constructed via one of the methods described herein and bearing a halogen or triflate at any of the 4 positions on the fused aryl ring may be substituted by a palladium mediated Suzuki-type coupling with a boronic acid or boronate ester. In the case where the arylamine group bears a protecting group, a deprotection step(s) may be required using acid, base and/or fluoride to give compounds of the present invention. In other cases, the new substituent may bear a protecting group which may be removed concomitantly or in a separate step. The new substituent may also contain an insaturation which may optionally be reduced. Depending on the nature of the arylamine, an atropisomeric mixture may be obtained. In such cases it may be necessary to isolate the atropisomer of interest to give compounds of the present invention. Alternatively, an atropisomerically enriched (e.g., pure) intermediate can be isolated and may be derivatized further to give compounds of the present invention.

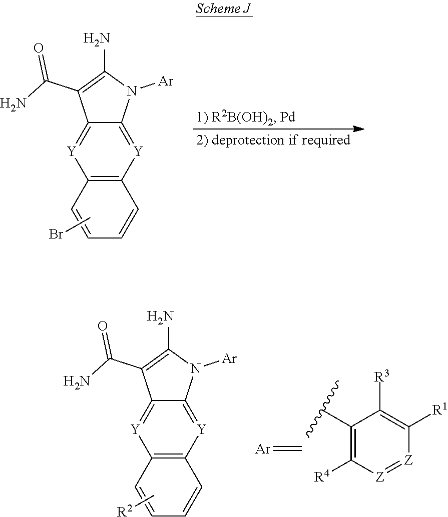

### Method K

Compounds of the present invention may be prepared as shown in Scheme K and described herein. The ester of a fused tricyclic system constructed via one of the methods described herein and bearing a carboxylate at any of the 4 positions on the fused phenyl ring may be hydrolyzed to the corresponding acid and coupled with an amine under amide forming conditions. Depending on the nature of the arylamine, a protecting group may be required to be in place prior to this reaction. In the case where the arylamine group bears a protecting group, a deprotection step(s) may be required using acid, base and/or fluoride to give compounds of the present invention. In other cases, the amine substituent may bear a protecting group which may be removed concomitantly or in a separate step. Depending on the nature of the arylamine, an atropisomeric mixture may be obtained. In such cases it may be necessary to isolate the atropisomer of interest to give compounds of the present invention. Alternatively, an atropisomerically enriched (e.g., pure) intermediate can be isolated and may be derivatized further to give compounds of the present invention.

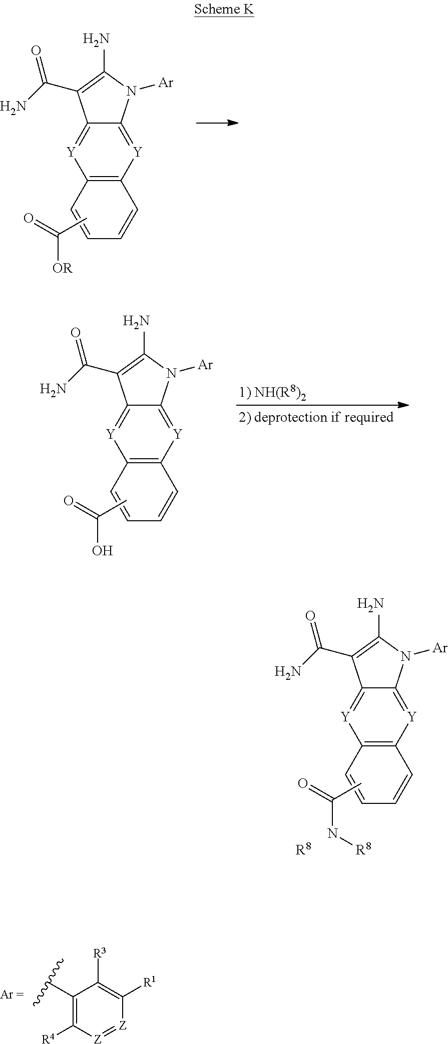

### Method L

Compounds of the present invention may be prepared as shown in Scheme L and described herein. The ester of a fused tricyclic system bearing a carboxylate at any of the 4 positions on the fused phenyl ring may react with a Grignard reagent to provide the corresponding tertiary alcohol. Depending on the nature of the arylamine, a protecting group may be required to be in place prior to this reaction. In the case where the arylamine group bears a protecting group, a deprotection step(s) may be required using acid, base and/or fluoride to give compounds of the present invention. The tertiary alcohol functional group may be dehydrated to the corresponding alkene during the deprotection steps. Depending on the nature of the arylamine, an atropisomeric mixture may be obtained. In such cases it may be necessary to isolate the atropisomer of interest to give compounds of the present invention. Alternatively, an atropisomerically enriched (e.g., pure) intermediate can be isolated and may be derivatized further to give compounds of the present invention.

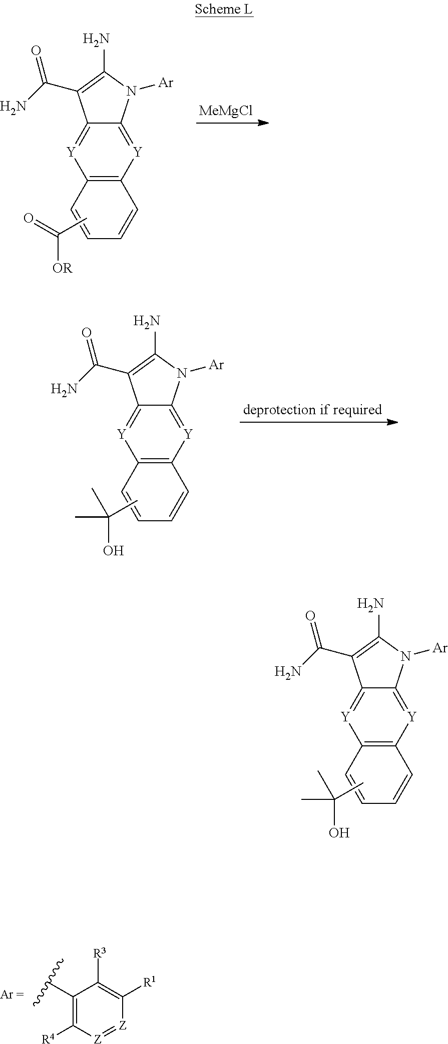

### Methods of Treatment

Compounds of the invention may be used for the treatment of a disease or condition (e.g., a cancer overexpressing CCNE1 or having an inactivating mutation in the FBXW7 gene) which depend on the activity of Myt1 (Gene name PKMYT1).

The disease or condition may have the symptom of cell hyperproliferation. For example, the disease or condition may be a cancer (e.g., a cancer overexpressing CCNE1 or having an inactivating mutation in the FBXW7 gene).

Cancers which have a high incidence of CCNE1 overexpression include e.g., uterine cancer, ovarian cancer, breast cancer, stomach cancer, esophageal cancer, lung cancer, and endometrial cancer.

Cancers which have a deficiency in FBXW7 include, e.g., uterine cancer, colorectal cancer, breast cancer, lung cancer, and esophageal cancer.

A compound of the invention may be administered by a route selected from the group consisting of oral, sublingual, buccal, transdermal, intradermal, intramuscular, parenteral, intravenous, intra-arterial, intracranial, subcutaneous, intraorbital, intraventricular, intraspinal, intraperitoneal, intranasal, inhalation, intratumoral, and topical administration.

### Pharmaceutical Compositions

The compounds used in the methods described herein are preferably formulated into pharmaceutical compositions for administration to human subjects in a biologically compatible form suitable for administration in vivo. Pharmaceutical compositions typically include a compound as described herein and a pharmaceutically acceptable excipient. Certain pharmaceutical compositions may include one or more additional pharmaceutically active agents described herein.

The compounds described herein can also be used in the form of the free base, in the form of salts, zwitterions, solvates, or as prodrugs, or pharmaceutical compositions thereof. All forms are within the scope of the invention. The compounds, salts, zwitterions, solvates, prodrugs, or pharmaceutical compositions thereof, may be administered to a patient in a variety of forms depending on the selected route of administration, as will be understood by those skilled in the art. The compounds used in the methods described herein may be administered, for example, by oral, parenteral, buccal, sublingual, nasal, rectal, patch, pump, or transdermal administration, and the pharmaceutical compositions formulated accordingly. Parenteral administration includes intravenous, intraperitoneal, subcutaneous, intramuscular, transepithelial, nasal, intrapulmonary, intrathecal, rectal, and topical modes of administration. Parenteral administration may be by continuous infusion over a selected period of time.

For human use, a compound of the invention can be administered alone or in admixture with a pharmaceutical carrier selected with regard to the intended route of administration and standard pharmaceutical practice. Pharmaceutical compositions for use in accordance with the present invention thus can be formulated in a conventional manner using one or more physiologically acceptable carriers including excipients and auxiliaries that facilitate processing of a compound of the invention into preparations which can be used pharmaceutically.

This invention also includes pharmaceutical compositions which can contain one or more pharmaceutically acceptable carriers. In making the pharmaceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, e.g., preservatives.

The excipient or carrier is selected on the basis of the mode and route of administration. Suitable pharmaceutical carriers, as well as pharmaceutical necessities for use in pharmaceutical formulations, are described in Remington: The Science and Practice of Pharmacy, 21st Ed., Gennaro, Ed., Lippincott Williams & Wilkins (2005), a well-known reference text in this field, and in the USP/NF (United States Pharmacopeia and the National Formulary). Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents, e.g., talc, magnesium stearate, and mineral oil; wetting agents; emulsifying and suspending agents; preserving agents, e.g., methyl- and propylhydroxy-benzoates; sweetening agents; and flavoring agents. Other exemplary excipients are described in Handbook of Pharmaceutical Excipients, 6th Edition, Rowe et al., Eds., Pharmaceutical Press (2009).

These pharmaceutical compositions can be manufactured in a conventional manner, e.g., by conventional mixing, dissolving, granulating, dragee-making, levigating, emulsifying, encapsulating, entrapping, or lyophilizing processes. Methods well known in the art for making formulations are found, for example, in Remington: The Science and Practice of Pharmacy, 21st Ed., Gennaro, Ed., Lippincott Williams & Wilkins (2005), and Encyclopedia of Pharmaceutical Technology, eds. J. Swarbrick and J. C. Boylan, 1988-1999, Marcel Dekker, New York. Proper formulation is dependent upon the route of administration chosen. The formulation and preparation of such compositions is well-known to those skilled in the art of pharmaceutical formulation. In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

### Dosages

The dosage of the compound used in the methods described herein, or pharmaceutically acceptable salts or prodrugs thereof, or pharmaceutical compositions thereof, can vary depending on many factors, e.g., the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine the appropriate dosage based on the above factors. The compounds used in the methods described herein may be administered initially in a suitable dosage that may be adjusted as required, depending on the clinical response. In general, a suitable daily dose of a compound of the invention will be that amount of the compound that is the lowest dose effective to produce a therapeutic effect. Such an effective dose will generally depend upon the factors described above.

A compound of the invention may be administered to the patient in a single dose or in multiple doses. When multiple doses are administered, the doses may be separated from one another by, for example, 1-24 hours, 1-7 days, 1-4 weeks, or 1-12 months. The compound may be administered according to a schedule or the compound may be administered without a predetermined schedule. An active compound may be administered, for example, 1, 2, 3, 4, 5, 6, 7, 8, 9, 10, 11, or 12 times per day, every 2nd, 3rd, 4th, 5th, or 6th day, 1, 2, 3, 4, 5, 6, or 7 times per week, 1, 2, 3, 4, 5, or 6 times per month, or 1, 2, 3, 4, 5, 6, 7, 8, 9, 10, 11, or 12 times per year. It is to be understood that, for any particular subject, specific dosage regimes should be adjusted overtime according to the individual need and the professional judgment of the person administering or supervising the administration of the compositions.

While the attending physician ultimately will decide the appropriate amount and dosage regimen, an effective amount of a compound of the invention may be, for example, a total daily dosage of, e.g., between 0.05 mg and 3000 mg of any of the compounds described herein. Alternatively, the dosage amount can be calculated using the body weight of the patient. Such dose ranges may include, for example, between 10-1000 mg (e.g., 50-800 mg). In some embodiments, 50, 100, 150, 200, 250, 300, 350, 400, 450, 500, 550, 600, 650, 700, 750, 800, 850, 900, 950, or 1000 mg of the compound is administered.

In the methods of the invention, the time period during which multiple doses of a compound of the invention are administered to a patient can vary. For example, in some embodiments, doses of the compounds of the invention are administered to a patient over a time period that is 1-7 days; 1-12 weeks; or 1-3 months. In some embodiments, the compounds are administered to the patient over a time period that is, for example, 4-11 months or 1-30 years. In some embodiments, the compounds are administered to a patient at the onset of symptoms. In any of these embodiments, the amount of compound that is administered may vary during the time period of administration. When a compound is administered daily, administration may occur, for example, 1, 2, 3, 4, 5, 6, 7, 8, 9, 10, 11, or 12 times per day.

Formulations

A compound identified as capable of treating any of the conditions described herein, using any of the methods described herein, may be administered to patients or animals with a pharmaceutically-acceptable diluent, carrier, or excipient, in unit dosage form. The chemical compounds for use in such therapies may be produced and isolated by any standard technique known to those in the field of medicinal chemistry. Conventional pharmaceutical practice may be employed to provide suitable formulations or compositions to administer the identified compound to patients suffering from a disease or condition. Administration may begin before the patient is symptomatic.

Exemplary routes of administration of the compounds (e.g., a compound of the invention), or pharmaceutical compositions thereof, used in the present invention include oral, sublingual, buccal, transdermal, intradermal, intramuscular, parenteral, intravenous, intra-arterial, intracranial, subcutaneous, intraorbital, intraventricular, intraspinal, intraperitoneal, intranasal, inhalation, and topical administration. The compounds desirably are administered with a pharmaceutically acceptable carrier. Pharmaceutical formulations of the compounds described herein formulated for treatment of the disorders described herein are also part of the present invention.

Formulations for Oral Administration

The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Formulations for oral administration may also be presented as chewable tablets, as hard gelatin capsules where the active ingredient is mixed with an inert solid diluent (e.g., potato starch, lactose, microcrystalline cellulose, calcium carbonate, calcium phosphate or kaolin), or as soft gelatin capsules where the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration versus time profile. In one example, controlled release is obtained by appropriate selection of various formulation parameters and ingredients, including, e.g., various types of controlled release compositions and coatings. Examples include single or multiple unit tablet or capsule compositions, oil solutions, suspensions, emulsions, microcapsules, microspheres, nanoparticles, patches, and liposomes. In some embodiments, compositions include biodegradable, pH, and/or temperature-sensitive polymer coatings.

Dissolution or diffusion-controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils, e.g., cottonseed oil, sesame oil, coconut oil, or peanut oil, as well as elixirs and similar pharmaceutical vehicles.

Formulations for Parenteral Administration

The compounds described herein for use in the methods of the invention can be administered in a pharmaceutically acceptable parenteral (e.g., intravenous or intramuscular) formulation as described herein. The pharmaceutical formulation may also be administered parenterally (intravenous, intramuscular, subcutaneous or the like) in dosage forms or formulations containing conventional, non-toxic pharmaceutically acceptable carriers and adjuvants. In particular, formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. For example, to prepare such a composition, the compounds of the invention may be dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution and isotonic sodium chloride solution. The aqueous formulation may also contain one or more preservatives, for example, methyl, ethyl, or n-propyl p-hydroxybenzoate. Additional information regarding parenteral formulations can be found, for example, in the United States Pharmacopeia-National Formulary (USP-NF), herein incorporated by reference.

The parenteral formulation can be any of the five general types of preparations identified by the USP-NF as suitable for parenteral administration:

(1) Drug Injection: a liquid preparation that is a drug substance (e.g., a compound of the invention), or a solution thereof;

(2) Drug for Injection: the drug substance (e.g., a compound of the invention) as a dry solid that will be combined with the appropriate sterile vehicle for parenteral administration as a drug injection;

(3) Drug Injectable Emulsion: a liquid preparation of the drug substance (e.g., a compound of the invention) that is dissolved or dispersed in a suitable emulsion medium;

(4) Drug Injectable Suspension: a liquid preparation of the drug substance (e.g., a compound of the invention) suspended in a suitable liquid medium; and

(5) Drug for Injectable Suspension: the drug substance (e.g., a compound of the invention) as a dry solid that will be combined with the appropriate sterile vehicle for parenteral administration as a drug injectable suspension.

Formulations for parenteral administration include solutions of the compound prepared in water suitably mixed with a surfactant, e.g., hydroxypropylcellulose. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Conventional procedures and ingredients for the selection and preparation of suitable formulations are described, for example, in Remington: The Science and Practice of Pharmacy, 21st Ed., Gennaro, Ed., Lippincott Williams & Wilkins (2005) and in The United States Pharmacopeia: The National Formulary (USP 36 NF31), published in 2013.

Formulations for parenteral administration may, for example, contain excipients, sterile water, or saline, polyalkylene glycols, e.g., polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The parenteral formulation can be formulated for prompt release or for sustained/extended release of the compound. Exemplary formulations for parenteral release of the compound include: aqueous solutions, powders for reconstitution, cosolvent solutions, oil/water emulsions, suspensions, oil-based solutions, liposomes, microspheres, and polymeric gels.

### Combinations

Compounds of the present invention may be administered to a subject in combination with one or more additional agents, e.g.:
(a) a cytotoxic agent;
(b) an antimetabolite;
(c) an alkylating agent;
(d) an anthracycline;
(e) an antibiotic;
(f) an anti-mitotic agent;
(g) a hormone therapy;
(h) a signal transduction inhibitor;
(i) a gene expression modulator;
(j) an apoptosis inducer;
(k) an angiogenesis inhibitor;
(l) an immunotherapy agent;
(m) a DNA damage repair inhibitor;
or
a combination thereof.

The cytotoxic agent may be, e.g., actinomycin-D, alemtuzumab, alitretinoin, allopurinol, altretamine, amifostine, amphotericin, amsacrine, arsenic trioxide, asparaginase, azacitidine, azathioprine, Bacille Calmette-Guerin (BCG), bendamustine, bexarotene, bevacuzimab, bleomycin, bortezomib, busulphan, capecitabine, carboplatin, carfilzomib, carmustine, cetuximab, cisplatin, chlorambucil, cladribine, clofarabine, colchicine, crisantaspase, cyclophosphamide, cyclosporine, cytarabine, cytochalasin B, dacarbazine, dactinomycin, darbepoetin alfa, dasatinib, daunorubicin, 1-dehydrotestosterone, denileukin, dexamethasone, dexrazoxane, dihydroxy anthracin dione, disulfiram, docetaxel, doxorubicin, emetine, epirubicin, erlotinib, epigallocatechin gallate, epoetin alfa, estramustine, ethidium bromide, etoposide, everolimus, filgrastim, finasunate, floxuridine, fludarabine, flurouracil (5-FU), fulvestrant, ganciclovir, geldanamycin, gemcitabine, glucocorticoids, gramicidin D, histrelin acetate, hydroxyurea, ibritumomab, idarubicin, ifosfamide, imatinib, irinotecan, interferons, interferon alfa-2a, interferon alfa-2b, ixabepilone, lactate dehydrogenase A (LDH-A), lenalidomide, letrozole, leucovorin, levamisole, lidocaine, lomustine, mechlorethamine, melphalan, 6-mercaptopurine, mesna, methotrexate, methoxsalen, metoprine, metronidazole, mithramycin, mitomycin-C, mitoxantrone, nandrolone, nelarabine, nilotinib, nofetumomab, oprelvekin, oxaliplatin, paclitaxel, pemetrexed, pentostatin, palifermin, pamidronate, pegademase, pegaspargase, pegfilgrastim, pemetrexed disodium, plicamycin, porfimer sodium, procaine, procarbazine, propranolol, puromycin, quinacrine, radicicol, radioactive isotopes, raltitrexed, rapamycin, rasburicase, salinosporamide A, sargramostim, sunitinib, temozolomide, teniposide, tetracaine, 6-thioguanine, thiotepa, topotecan, toremifene, trastuzumab, treosulfan, tretinoin, valrubicin, vinblastine, vincristine, vindesine, vinorelbine, zoledronate, or a combination thereof.

The antimetabolites may be, e.g., methotrexate, 6-mercaptopurine, 6-thioguanine, cytarabine, 5-fluorouracil decarbazine, cladribine, pemetrexed, gemcitabine, capecitabine, hydroxyurea, mercaptopurine, fludarabine, pralatrexate, clofarabine, cytarabine, decitabine, floxuridine, nelarabine, trimetrexate, thioguanine, pentostatin, or a combination thereof.

The alkylating agent may be, e.g., mechlorethamine, thiotepa, chlorambucil, melphalan, carmustine (BSNU), lomustine (CCNU), cyclothosphamide, busulfan, dibromomannitol, streptozotocin, mitomycin C, cis-dichlorodiamine platinum (II) (DDP) cisplatin, altretamine, cyclophosphamide, ifosfamide, hexamethylmelamine, altretamine, procarbazine, dacarbazine, temozolomide, streptozocin, carboplatin, cisplatin, oxaliplatin, uramustine, bendamustine, trabectedin, semustine, or a combination thereof.

The anthracycline may be, e.g., daunorubicin, doxorubicin, aclarubicin, aldoxorubicin, amrubicin, annamycin, carubicin, epirubicin, idarubicin, mitoxantrone, valrubicin, or a combination thereof.

The antibiotic may be, e.g., dactinomycin, bleomycin, mithramycin, anthramycin (AMC), ampicillin, bacampicillin, carbenicillin, cloxacillin, dicloxacillin, flucloxacillin, mezlocillin, nafcillin, oxacillin, piperacillin, pivampicillin, pivmecillinam, ticarcillin, aztreonam, imipenem, doripenem, ertapenem, meropenem, cephalosporins, clarithromycin, dirithromycin, roxithromycin, telithromycin, lincomycin, pristinamycin, quinupristin, amikacin, gentamicin, kanamycin, neomycin, netilmicin, paromomycin, tobramycin, streptomycin, sulfamethizole, sulfamethoxazole, sulfisoxazole, demeclocycline, minocycline, oxytetracycline, tetracycline, penicillin, amoxicillin, cephalexin, erythromycin, clarithromycin, azithromycin, ciprofloxacin, levofloxacin, ofloxacin, doxycycline, clindamycin, metronidazole, tigecycline, chloramphenicol, metronidazole, tinidazole, nitrofurantoin, vancomycin, teicoplanin, telavancin, linezolid, cycloserine, rifamycins, polymyxin B, bacitracin, viomycin, capreomycin, quinolones, daunorubicin, doxorubicin, 4′-deoxydoxorubicin, epirubicin, idarubicin, plicamycin, mitomycin-c, mitoxantrone, or a combination thereof.

The anti-mitotic agent may be, e.g., vincristine, vinblastine, vinorelbine, docetaxel, estramustine, ixabepilone, paclitaxel, maytansinoid, a dolastatin, a cryptophycin, or a combination thereof.

The signal transduction inhibitor may be, e.g., imatinib, trastuzumab, erlotinib, sorafenib, sunitinib, temsirolimus, vemurafenib, lapatinib, bortezomib, cetuximab panitumumab, matuzumab, gefitinib, STI 571, rapamycin, flavopiridol, imatinib mesylate, vatalanib, semaxinib, motesanib, axitinib, afatinib, bosutinib, crizotinib, cabozantinib, dasatinib, entrectinib, pazopanib, lapatinib, vandetanib, or a combination thereof.

The gene expression modulator may be, e.g., a siRNA, a shRNA, an antisense oligonucleotide, an HDAC inhibitor, or a combination thereof. An HDAC inhibitor may be, e.g., trichostatin A, trapoxin B, valproic acid, vorinostat, belinostat, LAQ824, panobinostat, entinostat, tacedinaline, mocetionstat, givinostat, resminostat, abexinostat, quisinostat, rocilinostat, practinostat, CHR-3996, butyric acid, phenylbutyric acid, 4SC202, romidepsin, sirtinol, cambinol, EX-527, nicotinamide, or a combination thereof. An antisense oligonucleotide may be, e.g., custirsen, apatorsen, AZD9150, trabadersen, EZN-2968, LErafAON-ETU, or a combination thereof. An siRNA may be, e.g., ALN-VSP, CALAA-01, Atu-027, SPC2996, or a combination thereof.

The hormone therapy may be, e.g., a luteinizing hormone-releasing hormone (LHRH) antagonist. The hormone therapy may be, e.g., firmagon, leuproline, goserelin, buserelin, flutamide, bicalutadmide, ketoconazole, aminoglutethimide, prednisone, hydroxyl-progesterone caproate, medroxy-progesterone acetate, megestrol acetate, diethylstil-bestrol, ethinyl estradiol, tamoxifen, testosterone propionate, fluoxymesterone, flutamide, raloxifene, droloxifene, iodoxyfene, 4-hydroxytamoxifen, trioxifene, keoxifene, LY117018, onapristone, toremifine citrate, megestrol acetate, exemestane, fadrozole, vorozole, letrozole, anastrozole, nilutamide, tripterelin, histerelin, arbiraterone, medroxyprogesterone acetate, diethylstilbestrol, premarin, fluoxymesterone, tretinoin, fenretinide, troxacitabine, or a combination thereof.

The apoptosis inducers may be, e.g., a recombinant human TNF-related apoptosis-inducing ligand (TRAIL), camptothecin, bortezomib, etoposide, tamoxifen, or a combination thereof.

The angiogenesis inhibitors may be, e.g., sorafenib, sunitinib, pazopanib, everolimus or a combination thereof.

The immunotherapy agent may be, e.g., a monoclonal antibody, cancer vaccine (e.g., a dendritic cell (DC) vaccine), oncolytic virus, cytokine, adoptive T cell therapy, Bacille Calmette-Guerin (BCG), GM-CSF, thalidomide, lenalidomide, pomalidomide, imiquimod, or a combination thereof. The monoclonal antibody may be, e.g., anti-CTLA4, anti-PD1, anti-PD-L1, anti-LAG3, anti-KIR, or a combination thereof.

The monoclonal antibody may be, e.g., alemtuzumab, trastuzumab, ibritumomab tiuxetan, brentuximab vedotin, trastuzumab, ado-trastuzumab emtansine, blinatumomab, bevacizumab, cetuximab, pertuzumab, panitumumab, ramucirumab, obinutuzumab, ofatumumab, rituximab, pertuzumab, tositumomab, gemtuzumab ozogamicin, tositumomab, or a combination thereof. The cancer vaccine may be, e.g., Sipuleucel-T, BioVaxlD, NeuVax, DCVax, SuVaxM, CIMAvax®, Provenge,®, hsp110 chaperone complex vaccine, CDX-1401, MIS416, CDX-110, GVAX Pancreas, HyperAcute™ Pancreas, GTOP-99 (MyVax®), or Imprime PGG®. The oncolytic virus may be, e.g., talimogene laherparepvec. The cytokine may be, e.g., IL-2, IFNα, or a combination thereof. The adoptive T cell therapy may be, e.g., tisagenlecleucel, axicabtagene ciloleucel, or a combination thereof.

The DNA damage repair inhibitor may be, e.g., a PARP inhibitor, a cell checkpoint kinase inhibitor, or a combination thereof. The PARP inhibitor may be, e.g., olaparib, rucaparib, veliparib (ABT-888), niraparib (ZL-2306), iniparib (BSI-201), talazoparib (BMN 673), 2X-121, CEP-9722, KU-0059436 (AZD2281), PF-01367338 or a combination thereof. The cell checkpoint kinase inhibitor may be, e.g., MK-1775 or AZD1775, AZD7762, LY2606368, PF-0477736, AZD0156, GDC-0575, ARRY-575, CCT245737, PNT-737 or a combination thereof.

### EXAMPLES

The following examples were meant to illustrate the invention. They were not meant to limit the invention in any way.

Reactions were typically performed at room temperature (rt or RT) under a nitrogen atmosphere using dry solvents (Sure/Seal™) if not described otherwise in the Examples below. Reactions were monitored by TLC or by injection of a small aliquot on a Waters Acquity-H UPLC® Class system using an Acquity® UPLC HSS C18 2.1×30 mm column eluting with a gradient (1.86 min) of acetonitrile (15% to 98%) in water (both containing 0.1% formic acid). Purifications by preparative HPLC were performed on a Teledyne Isco Combi Flash® EZ Prep system using either Phenomenex Gemini@ 5 μm NX-C18 110 Å 150×21.2 mm column at a flow of 40 mL/min over 12 min (<100 mg or multiple injections of <100 mg) or HP C18 RediSep® Rfgold column (>100 mg) eluting with an appropriate gradient of acetonitrile in water (both containing 0.1% formic acid) unless otherwise specified. The gradient was selected based on the retention time observed by reaction monitoring on the Waters Acquity-H UPLC® Class system (see above). Fractions containing the desired compounds were combined and finally lyophilized. Purifications by silica gel chromatography were performed on a Teledyne Isco Combi Flash® Rf system using RediSep® Rf silica gel columns of appropriate sizes. Purity of final Compounds was assessed by injection of a small aliquot on a Waters Acquity-H UPLC® Class system using an Acquity® UPLC BEH C18 2.1×50 mm column eluting with a gradient (7 min) of acetonitrile (2% to 98%) in water (both containing 0.1% formic acid).

### Example 1. Preparation of Compounds

### General Procedure 1 for Nitrile Hydrolysis

The nitrile intermediate was stirred in concentrated H₂SO₄ for an appropriate time to complete nitrile hydrolysis and concomitant removal of acid labile protecting groups such as benzyl, THP and PMB in some cases. The reaction mixture was then quenched with ice water and/or crushed ice and neutralized or made slightly alkaline with aqueous NH₄OH (7 or 14M). The solid formed was collected by filtration and washed with portions of H₂O. It was then air-dried then dried in vacuo. Alternatively, the product was extracted from the aqueous solution using DCM or EtOAc. When a subsequent deprotection step was needed, the crude product was often used as such. In other cases, the crude product was purified by prep HPLC, reverse phase flash chromatography on C18 cartridge or flash chromatography on silica gel as appropriate.

### General Procedure 2 for Methoxy Deprotection

To a solution of the appropriate methoxy phenyl intermediate in DCM was added excess tribromoborane (1M solution in DCM)—typically 3-6 eq. After stirring for the appropriate time at rt (sometime heating was required to complete deprotection), the reaction mixture was concentrated to dryness and coevaporated with DCM/MeOH and or MeOH a few times, then the residue was taken in MeOH, made basic with Et₃N and concentrated again. The residue was then purified by prep HPLC, reverse phase flash chromatography on C18 or flash chromatography on silica gel as appropriate.

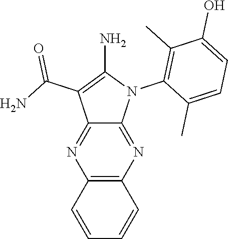 [SMILES: Cc1ccc(O)c(C)c1-n1c(N)c(C(N)=O)c2nc3ccccc3nc21]

### Compound 1 (2-amino-1-(3-hydroxy-2,6-dimethyl-phenyl)pyrrolo[3,2-b]quinoxaline-3-carboxamide), Compound 2 ((R)-2-amino-1-(3-hydroxy-2,6-dimethyl-phenyl)pyrrolo[3,2-b]quinoxaline-3-carboxamide) and Compound 3 ((S)-2-amino-1-(3-hydroxy-2,6-dimethyl-phenyl)pyrrolo[3,2-b]quinoxaline-3-carboxamide)

Step 1. Malononitrile (6.83 g, 104 mmol) was carefully added by portions with vigorous stirring to a suspension of sodium hydride (60% dispersion in mineral oil, 4.07 g, 106 mmol) in DME (200 mL). After the addition, the stirring was continued for 30 min. at RT and then 2,3-dichloroquinoxaline (10.16 g, 51.1 mmol) was added. The reaction mixture was stirred at RT for 30 min and then refluxed for 1 h. The DME was evaporated and cold aqueous 1M HCl was added to the resulting residue to give a precipitate that was filtered, washed with cold water and a minimum of cold ethanol to afford 2-(3-chloroquinoxalin-2-yl)propanedinitrile (6.70 g, 57% yield) as a yellow solid. ¹H NMR (400 MHz, DMSO-d6) δ 7.74-7.67 (m, 2H), 7.63-7.57 (m, 1H), 7.43-7.37 (m, 1H). MS: [M−1]: 227.0.

Step 2. A solution of 2-(3-chloroquinoxalin-2-yl)propanedinitrile (998 mg, 4.36 mmol) and 3-methoxy-2,6-dimethyl-aniline (arylamine AA1, 2.07 g, 13.7 mmol) in NMP (10 mL) was heated to 150° C. for 1 h. The reaction mixture was cooled down to RT, added dropwise to saturated NaHCO₃, stirred and extracted with EtOAc (3×). The combined organic layers were washed with brine, dried over Na₂SO₄, filtered and concentrated. The residue was adsorbed on silica using DCM and purified by flash chromatography on silica gel (0-100% EtOAc in hexanes). The relevant fractions were combined, concentrated and dried in vacuo to provide 2-amino-1-(3-methoxy-2,6-dimethyl-phenyl)pyrrolo[3,2-b]quinoxaline-3-carbonitrile (758 mg, 51% yield) as a dark orange solid. MS: [M+1]: 344.3.

Step 3. Following general procedure 1, treatment of 2-amino-1-(3-methoxy-2,6-dimethyl-phenyl)pyrrolo[3,2-b]quinoxaline-3-carbonitrile (130 mg, 0.379 mmol) with H₂SO₄ provided crude 2-amino-1-(3-methoxy-2,6-dimethyl-phenyl)pyrrolo[3,2-b]quinoxaline-3-carboxamide (135 mg, 99% yield) as an orange/brown solid which was used as such for the next step. MS: [M+1]: 362.2.

Step 4. Following general procedure 2, treatment of 2-amino-1-(3-methoxy-2,6-dimethyl-phenyl)pyrrolo[3,2-b]quinoxaline-3-carboxamide (784 mg, 2.17 mmol) with BBr₃ provided 2-amino-1-(3-hydroxy-2,6-dimethyl-phenyl)pyrrolo[3,2-b]quinoxaline-3-carboxamide (Compound 1, 619 mg, 82% yield) as an orange solid, after trituration of the residue in saturated NaHCO₃ and purification of the solid by flash chromatography on silica gel (1-20% MeOH in DCM). ¹H NMR (400 MHz, DMSO-d6) δ 9.65 (s, 1H), 7.99 (br s, 3H), 7.97-7.90 (m, 1H), 7.81-7.69 (m, 2H), 7.58 (ddd, J=8.4, 6.9, 1.5 Hz, 1H), 7.46 (ddd, J=8.3, 6.9, 1.5 Hz, 1H), 7.37 (br s, 1H), 7.12 (dt, J=8.3, 0.8 Hz, 1H), 6.98 (d, J=8.3 Hz, 1H), 1.83 (s, 3H), 1.75 (s, 3H). MS: [M+1]: 348.3.

Chiral SFC separation of 2-amino-1-(3-hydroxy-2,6-dimethyl-phenyl)pyrrolo[3,2-b]quinoxaline-3-carboxamide (619 mg), (Column: Chiral technologies IC, 10×250 mm, 5 μm; Conditions: Isocratic at 30% MeOH with 70% CO₂; Flow Rate: 10 mL/min; outlet pressure 100 bar) provided Compound 2 and Compound 3.

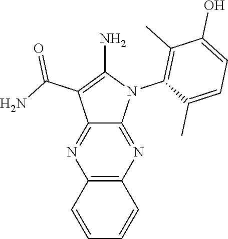

Compound 2 from chiral SFC separation of 2-amino-1-(3-hydroxy-2,6-dimethyl-phenyl)pyrrolo[3,2-b]quinoxaline-3-carboxamide. Peak 1 (7.67 min (chiral SFC), 99.9%). The fractions were concentrated then lyophilized from MeCN/H₂O, providing (R)-2-amino-1-(3-hydroxy-2,6-dimethyl-phenyl)pyrrolo[3,2-b]quinoxaline-3-carboxamide (222 mg). ¹H NMR (400 MHz, DMSO-d6) δ 9.65 (s, 1H), 7.99 (br s, 3H), 7.97-7.90 (m, 1H), 7.81-7.69 (m, 2H), 7.58 (ddd, J=8.4, 6.9, 1.5 Hz, 1H), 7.46 (ddd, J=8.3, 6.9, 1.5 Hz, 1H), 7.37 (br s, 1H), 7.12 (dt, J=8.3, 0.8 Hz, 1H), 6.98 (d, J=8.3 Hz, 1H), 1.83 (s, 3H), 1.75 (s, 3H). MS: [M+1]: 348.3.

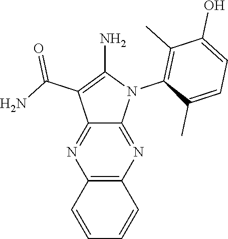

Compound 3 from chiral SFC separation of 2-amino-1-(3-hydroxy-2,6-dimethyl-phenyl)pyrrolo[3,2-b]quinoxaline-3-carboxamide. Peak 2 (11.20 min (chiral SFC), 99.7%). The fractions were concentrated then lyophilized from MeCN/H₂O, providing (S)-2-amino-1-(3-hydroxy-2,6-dimethyl-phenyl)pyrrolo[3,2-b]quinoxaline-3-carboxamide (227 mg). ¹H NMR (400 MHz, DMSO-d6) δ 9.65 (s, 1H), 7.99 (br s, 3H), 7.97-7.90 (m, 1H), 7.81-7.69 (m, 2H), 7.58 (ddd, J=8.4, 6.9, 1.5 Hz, 1H), 7.46 (ddd, J=8.3, 6.9, 1.5 Hz, 1H), 7.37 (br s, 1H), 7.12 (dt, J=8.3, 0.8 Hz, 1H), 6.98 (d, J=8.3 Hz, 1H), 1.83 (s, 3H), 1.75 (s, 3H). MS: [M+1]: 348.3.

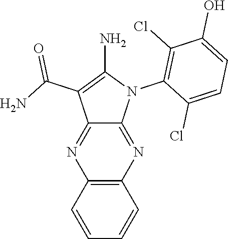 [SMILES: NC(=O)c1c(N)n(-c2c(Cl)ccc(O)c2Cl)c2nc3ccccc3nc12]

### Compound 15 (2-amino-1-(2,6-dichloro-3-hydroxy-phenyl)pyrrolo[3,2-b]quinoxaline-3-carboxamide)

Step 1. To a cold (0° C.) mixture 2,3-dichloroquinoxaline (98 mg, 0.492 mmol) and 2,6-dichloro-3-[(4-methoxyphenyl)methoxy]aniline (arylamine AA7, 292 mg, 0.979 mmol) in THE (4 mL) was added slowly a solution of potassium tert-butoxide in THE (1 M, 1.50 mL). After stirring for 1 h at 0° C., the reaction mixture was quenched with saturated NH₄Cl and diluted with EtOAc and H₂O. The layers were separated and the aqueous layer was extracted with EtOAc (2×). The combined organic extracts were washed with brine, dried over Na₂SO₄, filtered and concentrated. The residue was purified twice by flash chromatography on silica gel (0-50% EtOAc in hexanes). The desired fractions were combined, concentrated and dried in vacuo, affording 3-chloro-N-[2,6-dichloro-3-[(4-methoxyphenyl)methoxy]phenyl]quinoxalin-2-amine (109 mg, 48% yield) as a sticky off-white foam. ¹H NMR (400 MHz, Chloroform-d) δ 7.88 (ddd, J=8.3, 1.5, 0.6 Hz, 1H), 7.67 (ddd, J=8.4, 1.5, 0.7 Hz, 1H), 7.58 (ddd, J=8.4, 6.9, 1.5 Hz, 1H), 7.49 (ddd, J=8.4, 7.0, 1.5 Hz, 1H), 7.43-7.38 (m, 2H), 7.36 (d, J=9.0 Hz, 1H), 7.12 (s, 1H), 6.99-6.90 (m, 3H), 5.14 (s, 2H), 3.82 (s, 3H). MS: [M+1]: 462.0.

Step 2. To a MW vial containing sodium hydride (60% dispersion in mineral oil, 15 mg, 0.391 mmol) in dioxane (2 mL), was added a solution of malononitrile (17 mg, 0.26 mmol) in dioxane (0.5 mL). After 20 min, 3-chloro-N-[2,6-dichloro-3-[(4-methoxyphenyl)methoxy]phenyl]quinoxalin-2-amine (59 mg, 0.128 mmol) in dioxane (1 mL) and Pd(PPh₃)₄ (15 mg, 0.013 mmol) were added, the reaction mixture was flushed with N₂, the vial was capped and heated to 100° C. for 1 h. The reaction mixture was cooled to RT, quenched with saturated NH₄Cl, extracted with EtOAc (2×). The combined organic extracts were washed with brine, dried over Na₂SO₄, filtered and concentrated. The residue was purified by flash chromatography on silica gel (0-50% EtOAc in hexanes). The desired fractions were combined, concentrated and dried in vacuo, affording 2-amino-1-[2,6-dichloro-3-[(4-methoxyphenyl)methoxy]phenyl]pyrrolo[3,2-b]quinoxaline-3-carbonitrile (40 mg, 63% yield) as a yellow solid. MS: [M+1]: 490.1.

Step 3. Following general procedure 1, treatment of 2-amino-1-[2,6-dichloro-3-[(4-methoxyphenyl)methoxy]phenyl]pyrrolo[3,2-b]quinoxaline-3-carbonitrile (71 mg, 0.145 mmol) with H₂SO₄ provided 2-amino-1-(2,6-dichloro-3-hydroxy-phenyl)pyrrolo[3,2-b]quinoxaline-3-carboxamide (Compound 15, 29 mg, 52% yield) as a fluffy yellow solid, after precipitation from the reaction mixture and purification by prep HPLC (35-65% MeCN in H₂O, 0.1% formic acid modifier. ¹H NMR (400 MHz, DMSO-d6) δ 8.33 (br s, 2H), 7.95 (dd, J=8.3, 1.4 Hz, 1H), 7.79 (dd, J=8.3, 1.4 Hz, 1H), 7.74 (br s, J=3.1 Hz, 1H), 7.60 (ddd, J=8.4, 6.9, 1.5 Hz, 1H), 7.56 (d, J=9.0 Hz, 1H), 7.48 (ddd, J=8.4, 6.9, 1.5 Hz, 1H), 7.41 (br d, J=3.1 Hz, 1H), 7.26 (d, J=9.0 Hz, 1H). MS: [M+1]: 388.1.

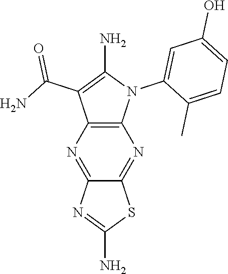 [SMILES: Cc1ccc(O)cc1-n1c(N)c(C(N)=O)c2nc3nc(N)sc3nc21]

### Compound 16 (2,6-diamino-5-(5-hydroxy-2-methylphenyl)-5H-pyrrolo[2,3-b]thiazolo[4,5-e]pyrazine-7-carboxamide)

Step 1. To a solution of 3,5-dibromo-6-chloro-pyrazin-2-amine (2.87 g, 9.99 mmol) in acetone (50 mL) was added 1-(isothiocyanatomethyl)-4-methoxy-benzene (2.30 g, 12.8 mmol, 2 mL), followed by sodium hydroxide (1.4 g, 35.0 mmol). The mixture was stirred at rt for 1 h, then it was neutralized with acetic acid and diluted with water. The mixture was stirred at rt for 20 min and filtered to collect the solid which was washed with water, dried in vacuo and purified by flash chromatography on silica gel (0-20% EtOAc in DCM) to provide 5-bromo-6-chloro-N-[(4-methoxyphenyl)methyl]thiazolo[4,5-b]pyrazin-2-amine (1.05 g, 27% yield) as an off-white solid. ¹H NMR (400 MHz, DMSO-d6) δ 9.59 (s, 1H), 7.27 (d, J=8.7 Hz, 2H), 6.98-6.76 (m, 2H), 4.56 (s, 2H), 3.68 (s, 3H). MS: [M+1]: 385.0.

Step 2. To a RBF under N₂ containing sodium hydride (60% dispersion in mineral oil, 220 mg, 5.74 mmol) and THE (15 mL) in an ice bath was added malononitrile (172 mg, 2.60 mmol) and the cold bath was removed. After stirring for 30 min, 5-bromo-6-chloro-N-[(4-methoxyphenyl)methyl]thiazolo[4,5-b]pyrazin-2-amine (500 mg, 1.30 mmol) and Pd(PPh₃)₄ (150 mg, 0.13 mmol) were added. The mixture was heated to 60° C. for 18 h. 4 g of silica was added, the mixture was evaporated and the residue was purified by flash chromatography on silica gel (0-15% MeOH in DCM) to provide 2-[6-chloro-2-[(4-methoxyphenyl)methylamino]thiazolo[4,5-b]pyrazin-5-yl]propanedinitrile (325 mg, 68% yield) as a yellow solid. MS: [M+1]: 371.1.

Step 3. Potassium tert-butoxide (182 mg, 1.62 mmol) was added to a solution of 2-[6-chloro-2-[(4-methoxyphenyl)methylamino]thiazolo[4,5-b]pyrazin-5-yl]propanedinitrile (500 mg, 1.35 mmol), 5-methoxy-2-methyl-aniline (225 mg, 1.64 mmol), Pd₂dba₃ (105 mg, 0.115 mmol), and Xantphos (133 mg, 0.230 mmol) in toluene (10 mL). The mixture was heated to reflux for 1 h. The solvent was evaporated and the brown residue was purified by flash chromatography on silica gel (0-20% EtOAc in hexanes) to provide 6-amino-5-(5-methoxy-2-methylphenyl)-2-((4-methoxybenzyl)amino)-5H-pyrrolo[2,3-b]thiazolo[4,5-e]pyrazine-7-carbonitrile (430 mg, 68% yield). MS: [M+1]: 472.1.

Step 4. Following general procedure 1, 6-amino-5-(5-methoxy-2-methylphenyl)-2-((4-methoxybenzyl)amino)-5H-pyrrolo[2,3-b]thiazolo[4,5-e]pyrazine-7-carbonitrile (550 mg, 1.17 mmol) was treated with H₂SO₄ to provide crude 2,6-diamino-5-(5-methoxy-2-methylphenyl)-5H-pyrrolo[2,3-b]thiazolo[4,5-e]pyrazine-7-carboxamide (405 mg, 94% yield) as a fluffy yellow solid after precipitation from the reaction mixture. MS: [M+1]: 370.2.

Step 5. Following general procedure 2, 2,6-diamino-5-(5-methoxy-2-methylphenyl)-5H-pyrrolo[2,3-b]thiazolo[4,5-e]pyrazine-7-carboxamide (405 mg, 1.10 mmol) was treated with BBr₃, to provide 2,6-diamino-5-(5-hydroxy-2-methylphenyl)-5H-pyrrolo[2,3-b]thiazolo[4,5-e]pyrazine-7-carboxamide (Compound 16, 26 mg, 7% yield) as a light yellow solid, after purification by prep HPLC (20-60% MeCN in H₂O, 0.1% formic acid modifier). ¹H NMR (400 MHz, DMSO-d6) δ 9.64 (s, 1H), 7.72 (s, 2H), 7.24 (dd, J=8.3, 0.8 Hz, 1H), 7.11 (s, 4H), 6.87 (dd, J=8.3, 2.6 Hz, 1H), 6.68 (d, J=2.5 Hz, 1H), 1.80 (s, 3H). MS: [M+1]: 356.1.

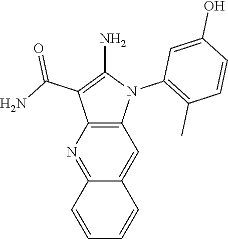 [SMILES: Cc1ccc(O)cc1-n1c(N)c(C(N)=O)c2nc3ccccc3cc21]

### Compound 17 (2-amino-1-(5-hydroxy-2-methylphenyl)-1H-pyrrolo[3,2-b]quinoline-3-carbonitrile)

Step 1. In a MW vial, 3-bromo-2-chloroquinoline (0.5 g, 2.066 mmol) and 5-(methoxymethoxy)-2-methylaniline (arylamine AA8, 0.24 g 1.445 mmol) was dissolved in toluene (10 mL) at room temperature followed by the addition of sodium tert-butoxide (0.23 g, 2.48 mmol). The reaction mixture was purged with N₂ gas, Pd₂(dba)₃ (0.15 g, 0.21 mmol) and Xantphos (0.12 g, 0.21 mmol) were added and again reaction mixture was purged with N₂ gas again, then submitted to microwave irradiation for 20 min at 110° C. The reaction mixture was quenched in water and extracted with EtOAc (3×). The combined organic layer was dried over Na₂SO₄, filtered and concentrated under vacuum to get crude product which was purified by flash chromatography on silica gel (5% EtOAc in hexanes). The pure product fractions were collected and evaporated to provide 2-chloro-N-(5-(methoxymethoxy)-2-methylphenyl)quinolin-3-amine (0.18 g, 27% yield).

Step 2. In a MW vial, malononitrile (54 mg, 0.82 mmol) was dissolved in DME (10 mL) at 0° C. followed by the addition of potassium tert-butoxide (0.36 g, 3.3 mmol). The reaction mixture was stirred at same temperature for 30 min, after which 2-chloro-N-(5-(methoxymethoxy)-2-methylphenyl)quinolin-3-amine (0.18 g, 0.55 mmol) was added and reaction mixture was submitted to microwave irradiation for 3 h at 150° C. The reaction mixture was quenched in water and extracted with EtOAc (3×). The combined organic layer was dried over Na₂SO₄, filtered and concentrated under vacuum to get crude product which was purified by flash chromatography on silica gel (70% EtOAc in hexanes). The pure product fractions were collected and evaporated to get of 2-amino-1-(5-(methoxymethoxy)-2-methylphenyl)-1H-pyrrolo[3,2-b]quinoline-3-carbonitrile (50 mg, 25% yield).

Step 3. 2-amino-1-(5-(methoxymethoxy)-2-methylphenyl)-1H-pyrrolo[3,2-b]quinoline-3-carbonitrile (50 mg, 0.14 mmol) was stirred in 4M HCl in dioxane (2 mL) for 3 h at room temperature. The reaction mixture was concentrated under vacuum to get the crude product which was triturated with n-pentane to obtain 2-amino-1-(5-hydroxy-2-methylphenyl)-1H-pyrrolo[3,2-b]quinoline-3-carbonitrile (HCl salt, 50 mg), which was used in next step without further purification.

Step 4. Following general procedure 1, treatment of 2-amino-1-(5-hydroxy-2-methylphenyl)-1H-pyrrolo[3,2-b]quinoline-3-carbonitrile (50 mg, 0.16 mmol) in H₂SO₄ at 0° C. provided 2-amino-1-(5-hydroxy-2-methylphenyl)-1H-pyrrolo[3,2-b]quinoline-3-carbonitrile (Compound 17, 3 mg, 6% yield) after aqueous work-up and purification by prep HPLC (SUNFIRE C18 250×19 mm, 5 μm, 0-100% MeCN in H₂O, 0.1% TFA modifier). ¹H NMR (500 MHz, DMSO-d6) δ 9.80 (s, 1H), 8.29 (d, J=3.7 Hz, 1H), 7.90 (d, J=8.5 Hz, 1H), 7.85 (dd, J=8.1, 1.4 Hz, 1H), 7.52 (br s+ddd, J=8.4, 6.8, 1.5 Hz, 3H), 7.36-7.31 (m, 2H), 7.29 (ddd, J=8.1, 6.8, 1.3 Hz, 1H), 7.18 (d, J=3.5 Hz, 1H), 6.96 (dd, J=8.4, 2.6 Hz, 1H), 6.79 (d, J=2.5 Hz, 1H), 1.87 (s, 3H). MS: [M+1]: 333.3.

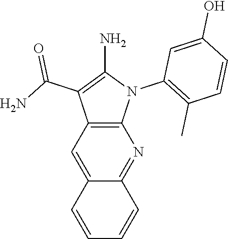 [SMILES: Cc1ccc(O)cc1-n1c(N)c(C(N)=O)c2cc3ccccc3nc21]

### Compound 18 (2-amino-1-(5-hydroxy-2-methylphenyl)-1H-pyrrolo[2,3-b]quinoline-3-carboxamide)

Step 1. In a MW vial, 3-bromo-2-chloroquinoline (0.2 g, 0.82 mmol) and 5-(methoxymethoxy)-2-methylaniline (arylamine AA8, 0.166 g, 0.99 mmol) was dissolved in toluene (2 mL) at room temperature followed by the addition of sodium tert-butoxide (94 mg, 0.98 mmol). The reaction mixture was purged with N₂ gas, Pd(OAc)₂ (28 mg, 0.04 mmol) and Xantphos (48 mg, 0.099 mmol) were added, purged with N₂ gas again, then submitted to microwave irradiation for 3 h at 85° C. The reaction mixture was filtered and the filtrate was concentrated under vacuum to get crude product, which was purified by flash chromatography on silica gel (0-2% EtOAc in hexanes). The pure product fractions were collected and evaporated to provide 3-bromo-N-(5-(methoxymethoxy)-2-methylphenyl)quinolin-2-amine (0.49 g, 16% yield).

Step 2. In a MW vial, malononitrile (14 mg 0.21 mmol) was dissolved in THE (3 mL) at 0° C. followed by the addition of sodium hydride (60% wt dispersion in mineral oil, 17 mg, 0.42 mmol). The reaction mixture was stirred at same temperature for 30 min, after which 3-bromo-N-(5-(methoxymethoxy)-2-methylphenyl)quinolin-2-amine (0.04 g, 0.10 mmol) and Pd(PPh₃)₄ (12 mg, 0.010 mmol) were added and the reaction mixture was submitted to microwave irradiation for 4 h at 80° C. The reaction mixture was diluted with water and extracted with EtOAc (3×). The combined organic layer was dried over Na₂SO₄, filtered and concentrated under vacuum to get crude product which was purified by flash chromatography on silica gel (20-50% EtOAc in hexanes). The pure product fractions were collected and evaporated to get 2-amino-1-(5-(methoxymethoxy)-2-methylphenyl)-1H-pyrrolo[2,3-b]quinoline-3-carbonitrile (39 mg, 25% yield).

Step 3. 2-amino-1-(5-(methoxymethoxy)-2-methylphenyl)-1H-pyrrolo[2,3-b]quinoline-3-carbonitrile (0.038 g, 0.10 mmol) was stirred in 3M HCl in MeOH (2 mL) for 3 h at rt. The reaction mixture was concentrated under vacuum to get the crude product which was triturated with n-pentane to obtain 2-amino-1-(5-hydroxy-2-methylphenyl)-1H-pyrrolo[2,3-b]quinoline-3-carbonitrile (HCl salt, 32 mg, 90% yield) which was used for next step without further purification.

Step 4. Following general procedure 1, treatment of 2-amino-1-(5-hydroxy-2-methylphenyl)-1H-pyrrolo[2,3-b]quinoline-3-carbonitrile (0.032 g, 0.10 mmol) in H₂SO₄ at 0° C. provided 2-amino-1-(5-hydroxy-2-methylphenyl)-1H-pyrrolo[2,3-b]quinoline-3-carboxamide (Compound 18, 6 mg, 17% yield) after aqueous work-up, purification by flash chromatography on silica gel (20-50% EtOAc in hexanes) and purification by prep HPLC (X-select Phenyl Hexyl (250×19) 5 μm, 0-100% MeCN in H₂O, 0.1% TFA modifier). ¹H NMR (500 MHz, DMSO-d6) δ 9.65 (s, 1H), 8.44 (s, 1H), 7.85 (dd, J=8.1, 1.5 Hz, 1H), 7.70 (d, J=8.3 Hz, 1H), 7.45 (br s, 2H), 7.45-7.41 (m, 1H), 7.38 (ddd, J=8.1, 6.7, 1.4 Hz, 1H), 7.29 (d, J=8.4 Hz, 1H), 6.92 (dd, J=8.4, 2.5 Hz, 1H), 6.86 (br s, 2H), 6.73 (d, J=2.6 Hz, 1H), 1.84 (s, 3H). MS: [M+1]: 333.2.

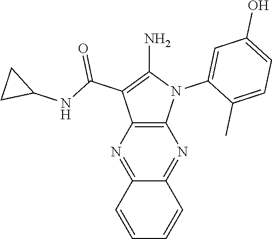 [SMILES: Cc1ccc(O)cc1-n1c(N)c(C(=O)NC2CC2)c2nc3ccccc3nc21]

### Compound 20 (2-amino-N-cyclopropyl-1-(5-hydroxy-2-methylphenyl)-1H-pyrrolo[2,3-b]quinoxaline-3-carboxamide)

Step 1. In a sealed tube, to a solution of 2,3-dichloroquinoxaline (3 g, 15 mmol) and 5-(methoxymethoxy)-2-methylaniline (arylamine AA8, 1.76 g, 10.55 mmol) in toluene (25 mL) was added sodium tert-butoxide (1.73 g, 18.1 mmol). The reaction mixture was purged with N₂ gas for 15 min, then Pd₂(dba)₃ (0.41 g, 0.45 mmol) and Xantphos (0.8 g, 1.5 mmol) were added. Then the reaction mixture was stirred at 110° C. for 16 h. After cooling down to rt, he reaction mixture was poured in water (90 mL) and extracted with EtOAc (3×). The combined organic layer was dried over Na₂SO₄, filtered and concentrated under vacuum to get the crude product which was purified by flash chromatography on silica gel (15% EtOAc in hexanes) The pure product fractions were collected and concentrated to get 3-chloro-N-(5-(methoxymethoxy)-2-methylphenyl) quinoxalin-2-amine (0.23 g, 4.63% yield). MS: [M+1]: 330.24.

Step 2. A MW vial containing 3-chloro-N-(5-(methoxymethoxy)-2-methylphenyl)quinoxalin-2-amine (0.15 g, 0.45 mmol), 2-cyano-N-cyclopropylacetamide (0.084 g 0.68 mmol) and Cs₂CO₃ (0.741 g, 2.27 mmol) in DMF (10 mL) was submitted to microwave irradiation for 1 h at 110° C. The reaction mixture was diluted with water and extracted with EtOAc (3×). The combined organic layer was dried over Na₂SO₄, filtered and concentrated under vacuum to get crude product which was purified by flash chromatography on silica gel (20% EtOAc in hexanes). The pure product fractions were collected and evaporated to get 2-amino-N-cyclopropyl-1-(5-(methoxymethoxy)-2-methylphenyl)-1H-pyrrolo[2,3-b]quinoxaline-3-carboxamide (0.1 g, 52% yield). MS: [M+1]: 418.34.

Step 3. To a cold (0° C.) solution of 2-amino-N-cyclopropyl-1-(5-(methoxymethoxy)-2-methylphenyl)-1H-pyrrolo[2,3-b]quinoxaline-3-carboxamide (0.1 g 0.2395 mmol in 1,4-dioxane (1 mL) at 0° C. was HCl in 1,4-dioxane (4M, 2 mL). The reaction mixture was stirred at rt for 30 min and the reaction mixture was concentrated under vacuum to get the crude product which was purified trituration with diethyl ether to get 2-amino-N-cyclopropyl-1-(5-hydroxy-2-methylphenyl)-1H-pyrrolo[2,3-b]quinoxaline-3-carboxamide (Compound 20, 55 mg, 68% yield). ¹H NMR (400 MHz, DMSO-d6) δ 9.75 (bs, 1H), 8.35 (s, 1H), 8.18 (bs, 1H), 8.00 (d, J=7.6 Hz, 1H), 7.80 (d, J=7.6 Hz, 1H), 7.60 (t, J=7.2 Hz, 1H), 7.49 (t, J=7.2 Hz, 1H), 7.31 (d, J=8.4 Hz, 1H), 6.96 (d, J=8.0 Hz, 1H), 6.83 (s, 1H), 2.88 (s, 1H), 1.89 (s, 3H), 0.82 (d, J=6.0 Hz, 2H), 0.67 (bs, 2H). MS: [M+1]: 374.32.

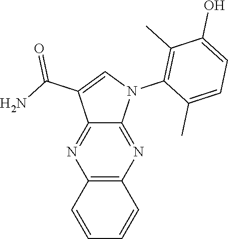 [SMILES: Cc1ccc(O)c(C)c1-n1cc(C(N)=O)c2nc3ccccc3nc21]

### Compound 24 (1-(3-hydroxy-2,6-dimethyl-phenyl)pyrrolo[3,2-b]quinoxaline-3-carboxamide)

Step 1. To a solution of 2-amino-1-(3-methoxy-2,6-dimethyl-phenyl)pyrrolo[3,2-b]quinoxaline-3-carbonitrile from Compound 1 step 2 (400 mg, 1.16 mmol) in THE (10 mL) was added tert-butyl nitrite (599 mg, 5.81 mmol). The reaction mixture was stirred at RT for 30 min then refluxed for 4.75 h, cooled down to room temperature and concentrated to dryness. The residue was purified by flash chromatography on silica (0-100% EtOAc in hexanes), providing 1-(3-methoxy-2,6-dimethyl-phenyl)pyrrolo[3,2-b]quinoxaline-3-carbonitrile (367 mg, 96% yield) as a light orange solid. MS: [M+1]: 329.3.

Step 2. Following general procedure 1, treatment of 1-(3-methoxy-2,6-dimethyl-phenyl)pyrrolo[3,2-b]quinoxaline-3-carbonitrile (367 mg, 1.12 mmol) with H₂SO₄ provided crude 1-(3-methoxy-2,6-dimethyl-phenyl)pyrrolo[3,2-b]quinoxaline-3-carboxamide (356 mg, 92% yield) as a beige solid which was used as such for the next step. MS: [M+1]: 347.3.

Step 3. Following general procedure 2, deprotection of 1-(3-methoxy-2,6-dimethyl-phenyl)pyrrolo[3,2-b]quinoxaline-3-carboxamide (356 mg, 1.03 mmol) afforded 1-(3-hydroxy-2,6-dimethyl-phenyl)pyrrolo[3,2-b]quinoxaline-3-carboxamide (Compound 24, 189 mg, 55% yield) as a beige fluffy solid, after purification by reverse phase flash chromatography on C18 cartridge (10-100% MeCN in H₂O, 0.1% formic acid modifier). ¹H NMR (400 MHz, DMSO-d6) δ 9.67 (s, 1H), 8.82 (s, 1H), 8.37-8.24 (m, 1H), 8.11-8.04 (m, 2H), 7.91-7.73 (m, 3H), 7.11 (dt, J=8.3, 0.7 Hz, 1H), 6.97 (d, J=8.3 Hz, 1H), 1.81 (s, 3H), 1.71 (s, 3H). MS: [M+1]: 333.4.

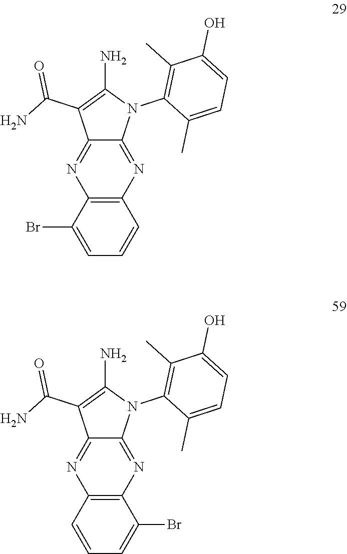 [SMILES: Cc1ccc(O)c(C)c1-n1c(N)c(C(N)=O)c2nc3c(Br)cccc3nc21 ; Cc1ccc(O)c(C)c1-n1c(N)c(C(N)=O)c2nc3cccc(Br)c3nc21]

### Compound 29 (2-amino-5-bromo-1-(3-hydroxy-2,6-dimethyl-phenyl)pyrrolo[2,3-b]quinoxaline-3-carboxamide) and Compound 59 (2-amino-8-bromo-1-(3-hydroxy-2,6-dimethyl-phenyl)pyrrolo[3,2-b]quinoxaline-3-carboxamide)

Step 1. Malononitrile (16.8 g, 254 mmol) was added portionwise to a vigorously stirred suspension of sodium hydride (60% dispersion in mineral oil, 10.3 g, 269 mmol) in DME (600 mL). After the addition, the stirring was continued for 30 min and then 5-bromo-2,3-dichloro-quinoxaline (intermediate R, 35.4 g, 127 mmol) was added (a small exotherm was observed). The reaction mixture was stirred at RT for 15 min and then heated under reflux for 4 h. The DME was evaporated and the resulting residue was poured by portion in cold aqueous 1M HCl to give a yellow precipitate that was filtered and washed with water to afford a mixture of 2-(5-bromo-3-chloro-quinoxalin-2-yl)propanedinitrile and 2-(8-bromo-3-chloro-quinoxalin-2-yl)propanedinitrile (36 g, 92% yield) (in about 1:1 ratio estimated by UPLCMS) as a yellow solid. MS: [M−1]: 306.9.

Step 2. 3-methoxy-2,6-dimethyl-aniline (arylamine AA1, 7.4 g, 48.9 mmol) was added to a mixture of 2-(5-bromo-3-chloro-quinoxalin-2-yl)propanedinitrile and 2-(8-bromo-3-chloro-quinoxalin-2-yl)propanedinitrile (5.00 g, 16.3 mmol) in NMP (50 mL). The reaction mixture was heated to 130° C. for 6 h, cooled, and the mixture was poured into vigorously stirring aqueous NaHCO₃ sat. The precipitate was collected by filtration, washed with water and dried by codistillation with toluene twice. The brown residue was taken in 250 mL of 15% MeOH in CH₂Cl₂ and 25 g of silica gel was added. The mixture was evaporated and the brown residue was purified by silica gel chromatography (dry load) using a gradient of 20 to 60% EtOAc in hexanes to provide a mixture of 2-amino-8-bromo-1-(3-methoxy-2,6-dimethyl-phenyl)pyrrolo[3,2-b]quinoxaline-3-carbonitrile and 2-amino-5-bromo-1-(3-methoxy-2,6-dimethyl-phenyl)pyrrolo[2,3-b]quinoxaline-3-carbonitrile (5.0 g, 73% yield) as an orange solid. MS: [M+1]: 424.1.

Step 3. Following general procedure 1, treatment of 2-amino-8-bromo-1-(3-methoxy-2,6-dimethyl-phenyl)pyrrolo[3,2-b]quinoxaline-3-carbonitrile and 2-amino-5-bromo-1-(3-methoxy-2,6-dimethyl-phenyl)pyrrolo[2,3-b]quinoxaline-3-carbonitrile (5.0 g, 11.8 mmol) with H₂SO₄ provided a crude mixture of 2-amino-8-bromo-1-(3-methoxy-2,6-dimethyl-phenyl)pyrrolo[3,2-b]quinoxaline-3-carboxamide and 2-amino-5-bromo-1-(3-methoxy-2,6-dimethyl-phenyl)pyrrolo[2,3-b]quinoxaline-3-carboxamide (5.2 g, quantitative yield) as a yellow solid, after precipitation from the reaction mixture. MS: [M+1]: 442.0.

Step 4. Following general procedure 2, deprotection of a mixture of 2-amino-8-bromo-1-(3-methoxy-2,6-dimethyl-phenyl)pyrrolo[3,2-b]quinoxaline-3-carboxamide and 2-amino-5-bromo-1-(3-methoxy-2,6-dimethyl-phenyl)pyrrolo[2,3-b]quinoxaline-3-carboxamide (2.4 g, 5.45 mmol) led to a mixture of 2-amino-8-bromo-1-(3-hydroxy-2,6-dimethyl-phenyl)pyrrolo[3,2-b]quinoxaline-3-carboxamide (1.8 g, 56% yield) as a red solid after purification by flash chromatography on silica gel (0-10% MeOH in DCM). The regiomers in the mixture were separated by SFC (Column: ZymorSPHER HA-Dipyridyl, 30×150 mm, 5 μm; Conditions: Isocratic at 50% MeOH+0.1% Formic Acid with 50% CO₂; Flow Rate: 70 mL/min; outlet pressure 100 bar) providing 2-amino-5-bromo-1-(3-hydroxy-2,6-dimethyl-phenyl)pyrrolo[2,3-b]quinoxaline-3-carboxamide (P1, Compound 29, RT 4.33 min, 550 mg, 46% yield)¹H NMR (400 MHz, DMSO-d6) δ 9.61 (s, 1H), 8.12 (m, 2H), 7.89 (dd, J=7.6, 1.3 Hz, 1H), 7.82 (d, J=3.3 Hz, 1H), 7.77 (dd, J=8.3, 1.3 Hz, 1H), 7.48 (d, J=3.3 Hz, 1H), 7.33 (dd, J=8.3, 7.6 Hz, 1H), 7.08 (dt, J=8.3, 0.8 Hz, 1H), 6.94 (d, J=8.3 Hz, 1H), 1.79 (s, 3H), 1.72 (s, 3H). MS: [M+1]: 428.0; and 2-amino-8-bromo-1-(3-hydroxy-2,6-dimethyl-phenyl)pyrrolo[3,2-b]quinoxaline-3-carboxamide (P2, Compound 59, RT 4.99 min, 250 mg, 22% yield). ¹H NMR (400 MHz, DMSO-d6) δ 9.68 (s, 1H), 8.12 (s, 2H), 7.96 (dd, J=8.3, 1.3 Hz, 1H), 7.80 (dd, J=7.6, 1.3 Hz, 1H), 7.70 (d, J=3.1 Hz, 1H), 7.49 (dd, J=8.3, 7.6 Hz, 1H), 7.44 (d, J=3.2 Hz, 1H), 7.13 (dt, J=8.3, 0.8 Hz, 1H), 6.98 (d, J=8.3 Hz, 1H), 1.86 (d, J=0.7 Hz, 3H), 1.78 (s, 3H). MS: [M+1]: 428.0. The regiochemistry was confirmed by Xray structure of Compound 29.

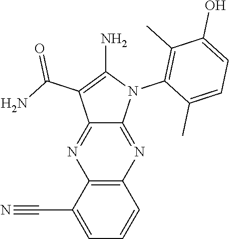 [SMILES: Cc1ccc(O)c(C)c1-n1c(N)c(C(N)=O)c2nc3c(C#N)cccc3nc21]

### Compound 32 (2-amino-5-cyano-1-(3-hydroxy-2,6-dimethyl-phenyl)pyrrolo[2,3-b]quinoxaline-3-carboxamide)

Copper (I) cyanide (32 mg, 0.36 mmol) was added to a solution of 2-amino-5-bromo-1-(3-hydroxy-2,6-dimethyl-phenyl)pyrrolo[2,3-b]quinoxaline-3-carboxamide (Compound 29, 50 mg, 0.117 mmol) in DMF (1 mL) and the suspension was stirred at 80° C. for 8 h. The mixture was filtered through a 0.45 micron PTFE filter and purified by preparative HPLC (30-80% MeCN in H₂O, 0.1% formic acid modifier). The recovered tubes were combined and lyophilized to provide 2-amino-5-cyano-1-(3-hydroxy-2,6-dimethyl-phenyl)pyrrolo[2,3-b]quinoxaline-3-carboxamide (Compound 32, 12 mg, 26% yield, 95% purity) as a pale yellow solid. ¹H NMR (400 MHz, DMSO-d6) δ 9.62 (s, 1H), 8.31 (s, 2H), 8.18-8.02 (m, 2H), 7.69 (d, J=3.1 Hz, 1H), 7.55 (d, J=6.7 Hz, 1H), 7.51 (dd, J=8.4, 7.3 Hz, 1H), 7.08 (d, J=8.3 Hz, 1H), 6.95 (d, J=8.3 Hz, 1H), 1.79 (s, 3H), 1.72 (s, 3H). MS: [M+1]: 373.2.

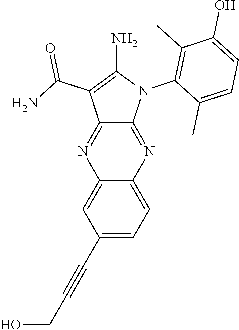 [SMILES: Cc1ccc(O)c(C)c1-n1c(N)c(C(N)=O)c2nc3cc(C#CCO)ccc3nc21]

### Compound 45 (2-amino-1-(3-hydroxy-2,6-dimethyl-phenyl)-6-(3-hydroxyprop-1-ynyl)pyrrolo[2,3-b]quinoxaline-3-carboxamide)

Diisopropylethylamine (163 mg, 1.26 mmol, 220 μL) was added to a mixture of 2-amino-6-bromo-1-(3-hydroxy-2,6-dimethyl-phenyl)pyrrolo[2,3-b]quinoxaline-3-carboxamide (Compound 38, 100 mg, 0.235 mmol), prop-2-yn-1-ol (31 mg, 0.55 mmol, 32 μL), Copper (I) iodide (12 mg, 0.063 mmol) and Pd(PPh₃)₄ (56 mg, 0.048 mmol) in acetonitrile (1.5 mL). The mixture was heated to 80° C. for 30 min. After cooling down to RT, the mixture was filtered on a 0.45 micron PTFE filter and the filtrate was purified by preparative HPLC (30-60% MeCN in H₂O, 0.1% formic acid modifier) The recovered tubes were combined and lyophilized to provide 2-amino-1-(3-hydroxy-2,6-dimethyl-phenyl)-6-(3-hydroxyprop-1-ynyl)pyrrolo[2,3-b]quinoxaline-3-carboxamide (Compound 45, 16 mg, 17% yield) as a pale yellow solid. ¹H NMR (400 MHz, DMSO-d6) δ 9.66 (s, 1H), 8.06 (s, 2H), 7.93 (d, J=1.8 Hz, 1H), 7.70 (d, J=8.5 Hz, 1H), 7.66 (d, J=3.2 Hz, 1H), 7.44-7.33 (m, 2H), 7.08 (d, J=8.3 Hz, 1H), 6.94 (d, J=8.3 Hz, 1H), 4.31 (s, 2H), 1.79 (s, 3H), 1.71 (s, 3H). MS: [M+1]: 402.4.

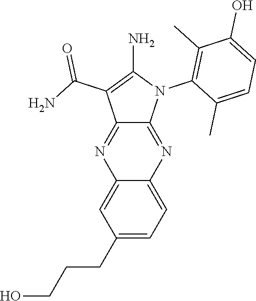 [SMILES: Cc1ccc(O)c(C)c1-n1c(N)c(C(N)=O)c2nc3cc(CCCO)ccc3nc21]

### Compound 46 (2-amino-1-(3-hydroxy-2,6-dimethyl-phenyl)-6-(3-hydroxypropyl)pyrrolo[2,3-b]quinoxaline-3-carboxamide)

A mixture of 2-amino-1-(3-hydroxy-2,6-dimethyl-phenyl)-6-(3-hydroxyprop-1-ynyl)pyrrolo[2,3-b]quinoxaline-3-carboxamide (Compound 45, 5 mg, 0.012 mmol) and palladium on carbon (10% w/w, 5 mg) in EtOH (2 mL) was stirred under H₂ atmosphere (balloon) for 2 h, the mixture was filtered on a 0.45 micron PTFE filter and the filtrate was evaporated to provide 2-amino-1-(3-hydroxy-2,6-dimethyl-phenyl)-6-(3-hydroxypropyl)pyrrolo[2,3-b]quinoxaline-3-carboxamide (Compound 46, 2 mg, 34% yield) as a pale yellow solid. ¹H NMR (400 MHz, DMSO-d6) δ 8.22-7.69 (m, 5H), 7.64 (dd, J=8.4, 1.5 Hz, 1H), 7.40-7.23 (m, 2H), 7.07 (d, J=8.3 Hz, 1H), 6.94 (dd, J=8.3, 1.4 Hz, 1H), 3.41 (t, J=6.4 Hz, 4H), 2.76 (t, J=7.6 Hz, 3H), 1.78 (d, J=6.1 Hz, 5H), 1.71 (d, J=1.5 Hz, 3H). MS: [M+1]: 406.2.

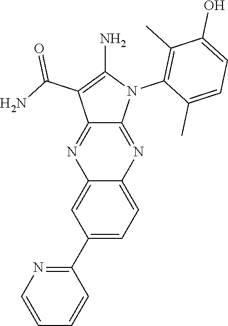 [SMILES: Cc1ccc(O)c(C)c1-n1c(N)c(C(N)=O)c2nc3cc(-c4ccccn4)ccc3nc21]

### Compound 49 (2-amino-1-(3-hydroxy-2,6-dimethyl-phenyl)-7-(3-pyridyl)pyrrolo[3,2-b]quinoxaline-3-carboxamide)

Pd(dppf)Cl₂.DCM (18 mg, 0.023 mmol) was added to a mixture of 2-amino-7-bromo-1-(3-hydroxy-2,6-dimethyl-phenyl)pyrrolo[3,2-b]quinoxaline-3-carboxamide (Compound 38, 100 mg, 0.235 mmol), 3-(4,4,5,5-tetramethyl-1,3,2-dioxaborolan-2-yl)pyridine (96 mg, 0.468 mmol), and aqueous K₂CO₃ (2 M, 350 μL) in DMF (0.8 mL). The mixture was heated to 80° C. for 2 h. After cooling to rt, the mixture was filtered on a 0.45 micron PTFE filter and purified by prep HPLC (20-60% MeCN in H₂O, 0.1% formic acid modifier). The recovered tubes were combined and lyophilized to provide 2-amino-1-(3-hydroxy-2,6-dimethyl-phenyl)-7-(3-pyridyl)pyrrolo[3,2-b]quinoxaline-3-carboxamide (Compound 49, 25 mg, 25% yield) as a pale yellow solid. ¹H NMR (400 MHz, DMSO-d6) δ 9.68 (s, 1H), 9.03 (dd, J=2.4, 0.8 Hz, 1H), 8.56 (dd, J=4.7, 1.6 Hz, 1H), 8.27 (dd, J=2.0, 0.6 Hz, 1H), 8.22 (m, 1H), 8.12-7.91 (m, 2H), 7.89-7.78 (m, 2H), 7.76 (d, J=3.0 Hz, 1H), 7.48 m, 1H), 7.45-7.35 (m, 1H), 7.09 (dt, J=8.3, 0.7 Hz, 1H), 6.96 (d, J=8.3 Hz, 1H), 1.81 (d, J=0.7 Hz, 3H), 1.73 (s, 3H). MS: [M+1]: 425.3.

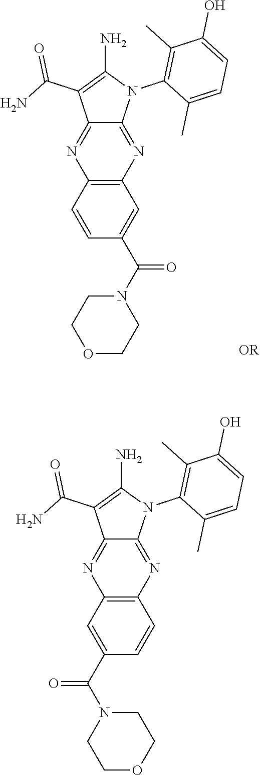 [SMILES: Cc1ccc(O)c(C)c1-n1c(N)c(C(N)=O)c2nc3ccc(C(=O)N4CCOCC4)cc3nc21 ; Cc1ccc(O)c(C)c1-n1c(N)c(C(N)=O)c2nc3cc(C(=O)N4CCOCC4)ccc3nc21]

### Compound 66 (2-amino-1-(3-hydroxy-2,6-dimethyl-phenyl)-7-(morpholine-4-carbonyl)pyrrolo[3,2-b]quinoxaline-3-carboxamide or 2-amino-1-(3-hydroxy-2,6-dimethyl-phenyl)-6-(morpholine-4-carbonyl)pyrrolo[3,2-b]quinoxaline-3-carboxamide)

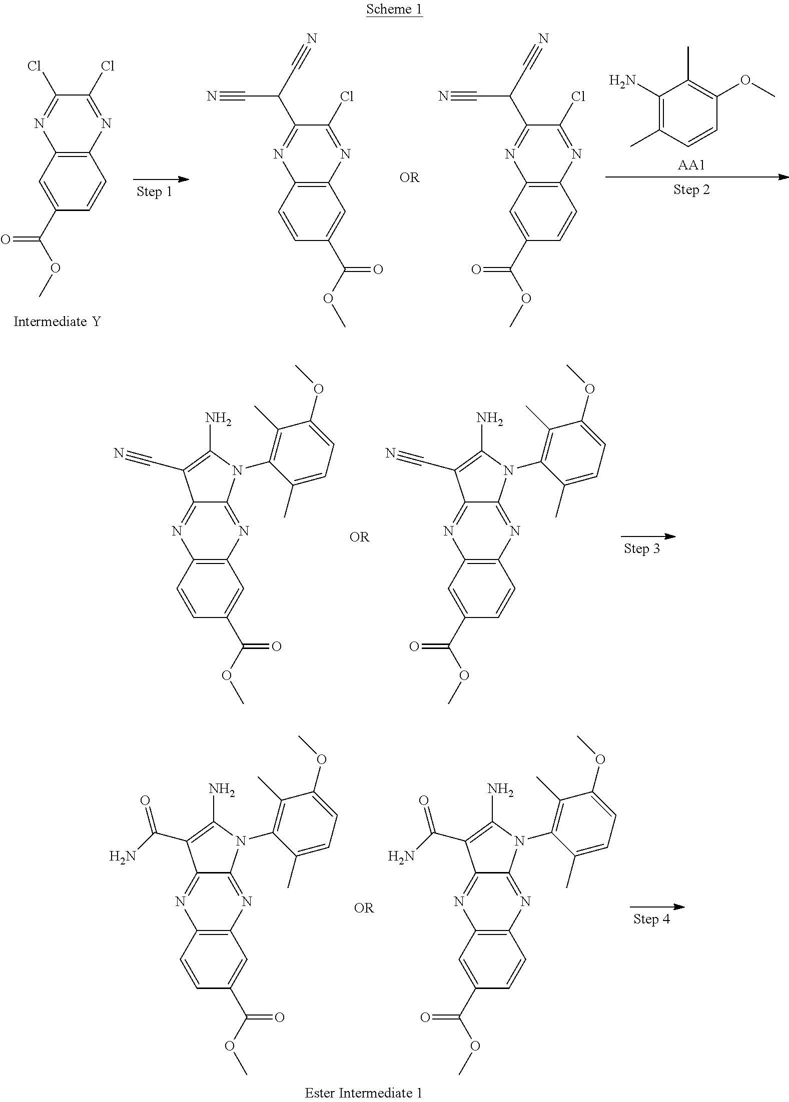

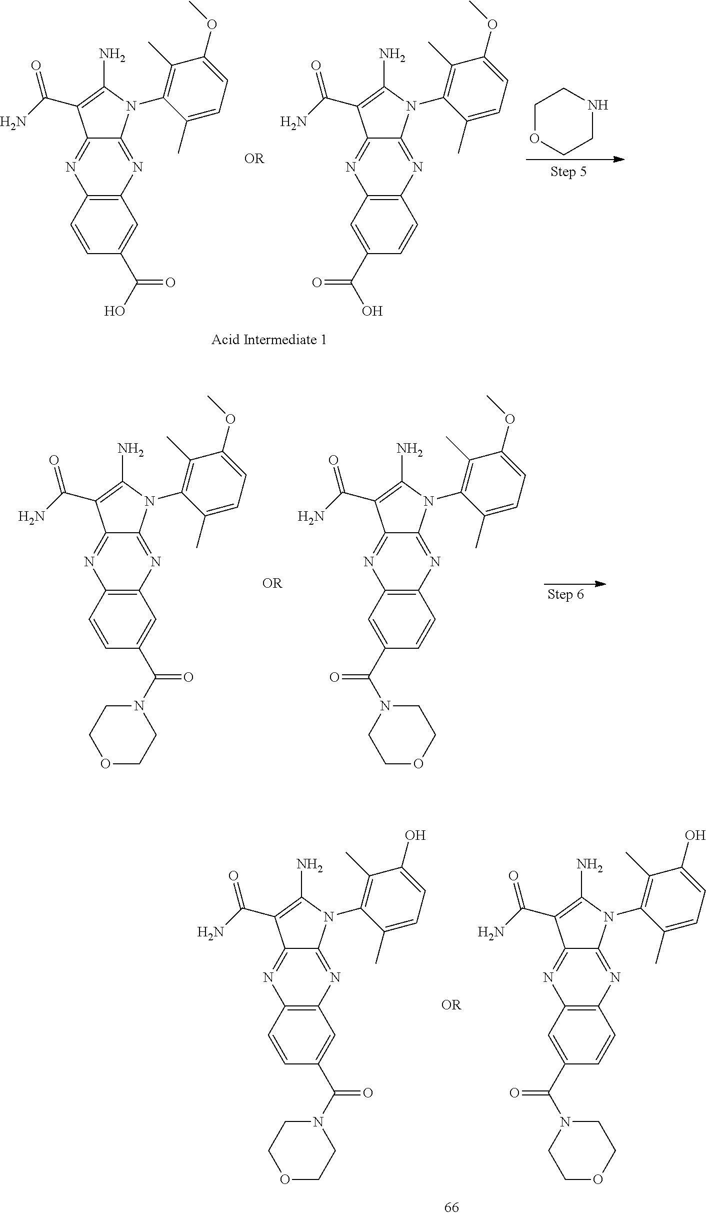

Steps 1, 2, 3. Applying the steps outlined in Method A and using Intermediate Y and arylamine AA1 and following general procedure 1 for hydrolysis of the nitrile, a single regioisomer of unknown regiochemistry which can be either 2-amino-3-carbamoyl-1-(3-methoxy-2,6-dimethyl-phenyl)pyrrolo[3,2-b]quinoxaline-7-carboxylate or 2-amino-3-carbamoyl-1-(3-methoxy-2,6-dimethyl-phenyl)pyrrolo[3,2-b]quinoxaline-6-carboxylate was obtained as a yellow solid (Ester Intermediate 1, 889 mg). ¹H NMR (400 MHz, DMSO-d6) δ 8.26 (dd, J=1.9, 0.6 Hz, 1H), 8.02 (dd, J=8.7, 1.9 Hz, 1H), 7.96 (dd, J=8.7, 0.5 Hz, 1H), 7.70 (br s, 1H), 7.45 (br s, 1H), 7.27 (d, J=7.7 Hz, 1H), 7.14 (d, J=8.5 Hz, 1H), 3.84 (s, 3H), 3.82 (s, 3H), 1.84 (s, 3H), 1.76 (s, 3H). MS: [M+1]: 420.2.

Step 4. To a suspension of 2-amino-3-carbamoyl-1-(3-methoxy-2,6-dimethyl-phenyl)pyrrolo[3,2-b]quinoxaline-7-carboxylate or 2-amino-3-carbamoyl-1-(3-methoxy-2,6-dimethyl-phenyl)pyrrolo[3,2-b]quinoxaline-6-carboxylate (Ester Intermediate 1, 443 mg, 1.06 mmol) in MeOH (6 mL) and THE (9 mL) was added aqueous LiOH (1 M, 3.2 mL) and the mixture was stirred overnight, more aqueous LiOH (1 M, 1 mL) was added and the reaction was allowed to continue for another 5 days. The reaction mixture was concentrated to remove volatiles, acidified to pH 4 with 1N HCl and the solid formed was collected by filtration, washed with H₂O, air-dried then dried in vacuo, affording crude product as an orange-brown solid (413 mg, 96% yield). The product consists of a single regioisomer that is 2-amino-3-carbamoyl-1-(3-methoxy-2,6-dimethyl-phenyl)pyrrolo[3,2-b]quinoxaline-7-carboxylic acid or 2-amino-3-carbamoyl-1-(3-methoxy-2,6-dimethyl-phenyl)pyrrolo[3,2-b]quinoxaline-6-carboxylic acid (Acid Intermediate 1). MS: [M+1]: 406.4.

Step 5. To a vial charged with 2-amino-3-carbamoyl-1-(3-methoxy-2,6-dimethyl-phenyl)pyrrolo[3,2-b]quinoxaline-7-carboxylic acid or 2-amino-3-carbamoyl-1-(3-methoxy-2,6-dimethyl-phenyl)pyrrolo[3,2-b]quinoxaline-6-carboxylic acid (Acid Intermediate 1, 45 mg, 0.11 mmol) and HATU (46 mg, 0.12 mmol) was added DMF (0.5 mL), morpholine (13 mg, 0.15 mmol, 13 μL) then DIPEA (43 mg, 0.33 mmol, 58 μL). The mixture was stirred for 45 min, diluted with H₂O (3 mL) and the resulting solid was collected by filtration and washed with H₂O, air-dried then dried in vacuo, affording a pale yellow solid (37 mg, 70% yield). The product consists of a single regioisomer that is 2-amino-1-(3-methoxy-2,6-dimethyl-phenyl)-7-(morpholine-4-carbonyl)pyrrolo[3,2-b]quinoxaline-3-carboxamide or 2-amino-1-(3-methoxy-2,6-dimethyl-phenyl)-6-(morpholine-4-carbonyl)pyrrolo[3,2-b]quinoxaline-3-carboxamide. MS: [M+1]: 475.4.

Step 6. Following general procedure 2, treatment of 2-amino-1-(3-methoxy-2,6-dimethyl-phenyl)-7-(morpholine-4-carbonyl)pyrrolo[3,2-b]quinoxaline-3-carboxamide or 2-amino-1-(3-methoxy-2,6-dimethyl-phenyl)-6-(morpholine-4-carbonyl)pyrrolo[3,2-b]quinoxaline-3-carboxamide (37 mg, 0.078 mmol) with BBr₃ led to a pale yellow fluffy solid (17 mg, 47% yield) after purification by prep HPLC (25-55% MeCN in H₂O, 0.1% formic acid modifier). The product consists of a single regioisomer that is 2-amino-1-(3-hydroxy-2,6-dimethyl-phenyl)-7-(morpholine-4-carbonyl)pyrrolo[3,2-b]quinoxaline-3-carboxamide or 2-amino-1-(3-hydroxy-2,6-dimethyl-phenyl)-6-(morpholine-4-carbonyl)pyrrolo[3,2-b]quinoxaline-3-carboxamide (Compound 66). ¹H NMR (400 MHz, DMSO-d6) δ 9.69 (s, 1H), 8.12 (brs, 2H), 7.98 (d, J=8.5 Hz, 1H), 7.79 (d, J=1.8 Hz, 1H), 7.74 (br d, J=3.3 Hz, 1H), 7.59 (dd, J=8.5, 1.9 Hz, 1H), 7.43 (br s, 1H), 7.13 (dt, J=8.3, 0.8 Hz, 1H), 6.98 (d, J=8.3 Hz, 1H), 3.55 (br m, 8H), 1.83 (s, 3H), 1.76 (s, 3H). MS: [M+1]: 461.4.

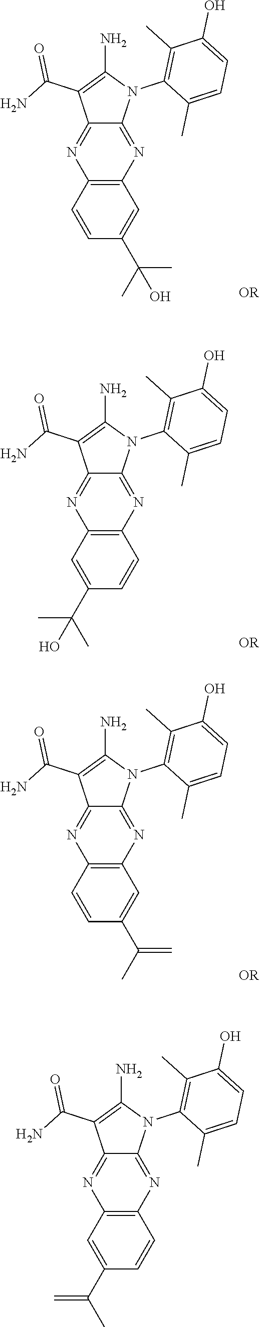 [SMILES: Cc1ccc(O)c(C)c1-n1c(N)c(C(N)=O)c2nc3ccc(C(C)(C)O)cc3nc21 ; Cc1ccc(O)c(C)c1-n1c(N)c(C(N)=O)c2nc3cc(C(C)(C)O)ccc3nc21 ; C=C(C)c1ccc2nc3c(C(N)=O)c(N)n(-c4c(C)ccc(O)c4C)c3nc2c1 ; C=C(C)c1ccc2nc3c(nc2c1)c(C(N)=O)c(N)n3-c1c(C)ccc(O)c1C]

### Compound 67 (2-amino-1-(3-hydroxy-2,6-dimethyl-phenyl)-7-(1-hydroxy-1-methyl-ethyl)pyrrolo[3,2-b]quinoxaline-3-carboxamide or 2-amino-1-(3-hydroxy-2,6-dimethyl-phenyl)-6-(1-hydroxy-1-methyl-ethyl)pyrrolo[3,2-b]quinoxaline-3-carboxamide) and Compound 68 (2-amino-1-(3-hydroxy-2,6-dimethyl-phenyl)-7-isopropenyl-pyrrolo[3,2-b]quinoxaline-3-carboxamide or either 2-amino-1-(3-hydroxy-2,6-dimethyl-phenyl)-6-isopropenyl-pyrrolo[3,2-b]quinoxaline-3-carboxamide)

Step 1. To a solution of 2-amino-3-carbamoyl-1-(3-methoxy-2,6-dimethyl-phenyl)pyrrolo[3,2-b]quinoxaline-7-carboxylate or 2-amino-3-carbamoyl-1-(3-methoxy-2,6-dimethyl-phenyl)pyrrolo[3,2-b]quinoxaline-6-carboxylate (Ester Intermediate 1, 82 mg, 0.20 mmol) in THE (2.5 mL) at −40° C. was added methylmagnesium chloride in THE (3 M, 655 μL) dropwise. The reaction mixture was stirred under N₂, allowing to warm up to rt and stirred overnight. It was then quenched with saturated aqueous NH₄Cl and diluted with EtOAc. The layers separated and the aqueous layer was extracted with EtOAc (2×). The combined organic extracts were washed with brine, dried over Na₂SO₄, filtered and concentrated. The crude product was adsorbed on silica and purified by flash chromatography on silica gel (50-100% EtOAc in hexanes). The appropriate fractions were combined, concentrated and dried in vacuo to provide a yellow solid (54 mg, 66% yield) which was not pure but was carried through the next step without further purification. The product consists of a single regioisomer that is 2-amino-7-(1-hydroxy-1-methyl-ethyl)-1-(3-methoxy-2,6-dimethyl-phenyl)pyrrolo[3,2-b]quinoxaline-3-carboxamide or 2-amino-6-(1-hydroxy-1-methyl-ethyl)-1-(3-methoxy-2,6-dimethyl-phenyl)pyrrolo[3,2-b]quinoxaline-3-carboxamide. MS: [M+1]: 420.3.

Step 2. Following general procedure 2, treatment of 2-amino-3-carbamoyl-1-(3-methoxy-2,6-dimethyl-phenyl)pyrrolo[3,2-b]quinoxaline-7-carboxylate or 2-amino-3-carbamoyl-1-(3-methoxy-2,6-dimethyl-phenyl)pyrrolo[3,2-b]quinoxaline-6-carboxylate (54 mg, 0.13 mmol) with BBr₃ led to the final product (5 mg, 10% yield) as a pale yellow fluffy solid after purification by prep HPLC (30-60% MeCN in H₂O, 0.1% formic acid modifier). The product consists of a single regioisomer that is 2-amino-1-(3-hydroxy-2,6-dimethyl-phenyl)-7-(1-hydroxy-1-methyl-ethyl)pyrrolo[3,2-b]quinoxaline-3-carboxamide or 2-amino-1-(3-hydroxy-2,6-dimethyl-phenyl)-6-(1-hydroxy-1-methyl-ethyl)pyrrolo[3,2-b]quinoxaline-3-carboxamide (Compound 67). ¹H NMR (400 MHz, DMSO-d6) δ 9.66 (s, 1H), 8.03-7.90 (m, 2H), 7.88 (dd, J=8.6, 0.5 Hz, 1H), 7.77 (dd, J=2.2, 0.5 Hz, 1H), 7.76-7.66 (m, 2H), 7.34 (br s, 1H), 7.12 (d, J=8.3 Hz, 1H), 6.98 (d, J=8.3 Hz, 1H), 5.09 (s, 1H), 1.83 (s, 3H), 1.75 (s, 3H), 1.48 (s, 6H). MS: [M+1]: 406.5. Upon dissolving the crude product in a mixture of MeCN/H₂O/dmso for the purification by prep HPLC, the formation of a dehydration side product was observed. This side-product was also isolated and repurified by prep HPLC 50-80% MeCN in H₂O, 0.1% formic acid modifier) to afford a single regioisomer that is 2-amino-1-(3-hydroxy-2,6-dimethyl-phenyl)-7-isopropenyl-pyrrolo[3,2-b]quinoxaline-3-carboxamide or 2-amino-1-(3-hydroxy-2,6-dimethyl-phenyl)-6-isopropenyl-pyrrolo[3,2-b]quinoxaline-3-carboxamide (Compound 68, 13 mg, 26% yield) as a pale yellow fluffy solid. ¹H NMR (400 MHz, DMSO-d6) δ 9.66 (s, 1H), 8.01 (br s, 2H), 7.89 (dd, J=7.8, 1.8 Hz, 1H), 7.85-7.78 (m, 2H), 7.73 (d, J=3.2 Hz, 1H), 7.38 (d, J=3.2 Hz, 1H), 7.12 (dt, J=8.3, 0.8 Hz, 1H), 6.98 (d, J=8.3 Hz, 1H), 5.60 (s, 1H), 5.16 (t, J=1.5 Hz, 1H), 2.18 (s, 3H), 1.83 (s, 3H), 1.75 (s, 3H). MS: [M+1]: 388.4.

### Preparation of Ester Intermediate 2 and Acid Intermediate 2

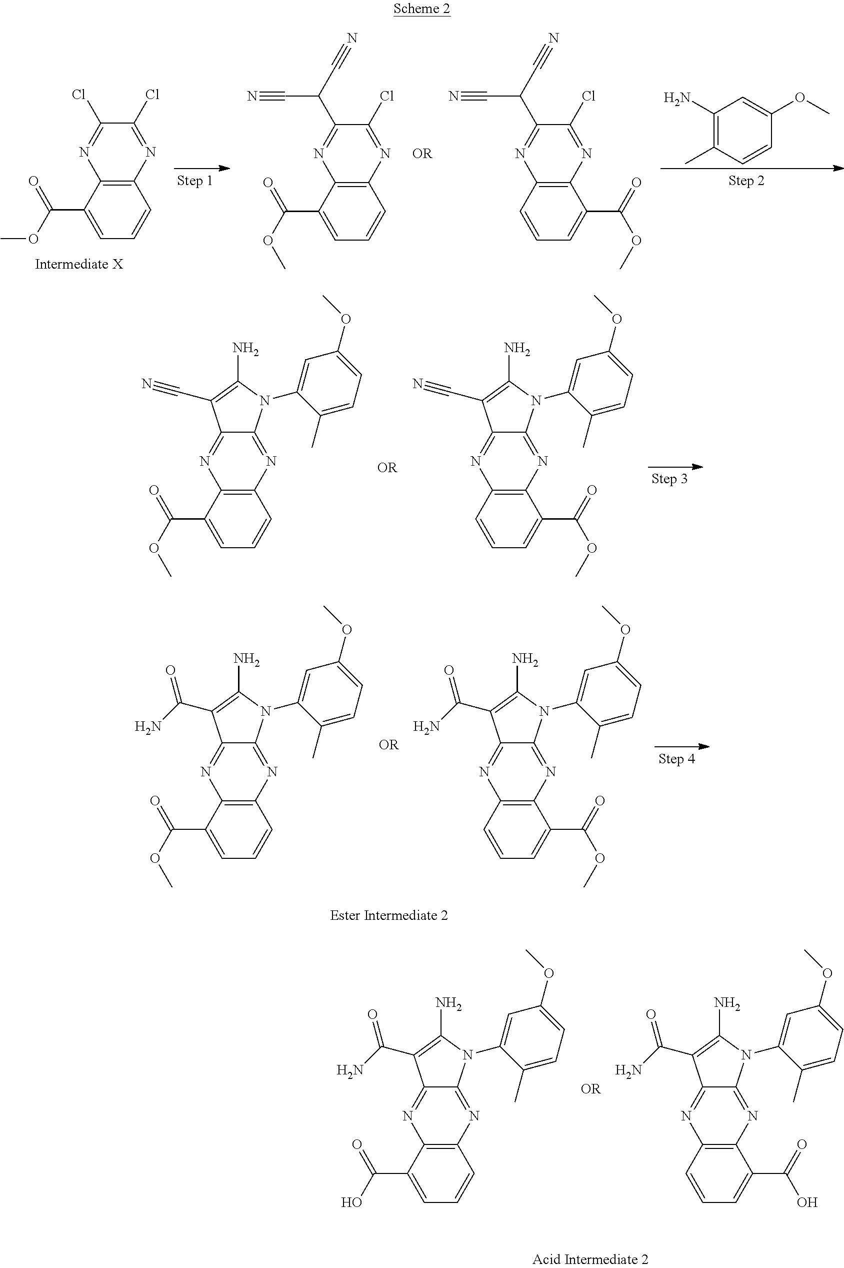

Steps 1, 2, 3. Applying the steps outlined in Method A and using Intermediate X and 5-methoxy-2-methyl-aniline and following general procedure 1 for hydrolysis of the nitrile, a major regioisomer of unknown regiochemistry which can be either methyl 2-amino-3-carbamoyl-1-(5-methoxy-2-methyl-phenyl)pyrrolo[2,3-b]quinoxaline-5-carboxylate or methyl 2-amino-3-carbamoyl-1-(5-methoxy-2-methyl-phenyl)pyrrolo[2,3-b]quinoxaline-8-carboxylate was obtained as a dark yellow solid, (Ester Intermediate 2, 1.78 g). ¹H NMR (400 MHz, DMSO-d6) δ 7.95 (dd, J=8.2, 1.5 Hz, 1H), 7.93-7.87 (m, 2H), 7.50 (dd, J=8.3, 7.3 Hz, 1H), 7.43 (dt, J=8.2, 0.7 Hz, 1H), 7.37 (br d, J=3.3 Hz, 1H), 7.16-7.09 (m, 2H), 3.92 (s, 3H), 3.78 (s, 3H), 1.94 (s, 3H). MS: [M+1]: 406.2.

Step 4. To a solution of methyl 2-amino-3-carbamoyl-1-(5-methoxy-2-methyl-phenyl)pyrrolo[2,3-b]quinoxaline-5-carboxylate or methyl 2-amino-3-carbamoyl-1-(5-methoxy-2-methyl-phenyl)pyrrolo[2,3-b]quinoxaline-8-carboxylate (300 mg, 0.740 mmol) in THE (4.5 mL) and MeOH (3 mL) was added aqueous lithium hydroxide (1 M, 1.5 mL) and the resulting mixture was stirred for 24 at rt, more aqueous lithium hydroxide (1 M, 1.5 mL) was added and the mixture was stirred for another 6 h. The reaction mixture was concentrated to remove the volatiles, acidified to pH 4-5 with aqueous 1N HCl and the resulting solid collected by filtration and washed with H₂O, air-dried then dried in vacuo to afford crude product as a yellow solid (Acid Intermediate 2, 259 mg, 89% yield), consisting of a single regioisomer that is 2-amino-3-carbamoyl-1-(5-methoxy-2-methyl-phenyl)pyrrolo[2,3-b]quinoxaline-5-carboxylic acid or 2-amino-3-carbamoyl-1-(5-methoxy-2-methyl-phenyl)pyrrolo[2,3-b]quinoxaline-8.-carboxylic acid. ¹H NMR (400 MHz, DMSO-d6) δ 8.31 (br s, 2H), 8.03 (dd, J=7.3, 1.5 Hz, 1H), 7.97 (dd, J=8.3, 1.5 Hz, 1H), 7.53 (dd, J=8.3, 7.3 Hz, 1H), 7.47 (br s, 3H), 7.43 (dt, J=8.1, 0.8 Hz, 1H), 7.19-7.07 (m, 2H), 3.78 (s, 3H), 1.95 (s, 3H). MS: [M+1]: 392.2.

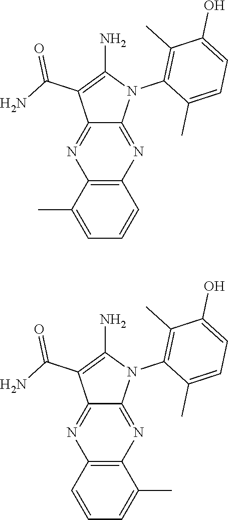 [SMILES: Cc1ccc(O)c(C)c1-n1c(N)c(C(N)=O)c2nc3c(C)cccc3nc21 ; Cc1ccc(O)c(C)c1-n1c(N)c(C(N)=O)c2nc3cccc(C)c3nc21]

### Compound 77 and Compound 78 (2-amino-1-(3-hydroxy-2,6-dimethyl-phenyl)-5-methyl-pyrrolo[2,3-b]quinoxaline-3-carboxamide and 2-amino-1-(3-hydroxy-2,6-dimethyl-phenyl)-8-methyl-pyrrolo[3,2-b]quinoxaline-3-carboxamide)

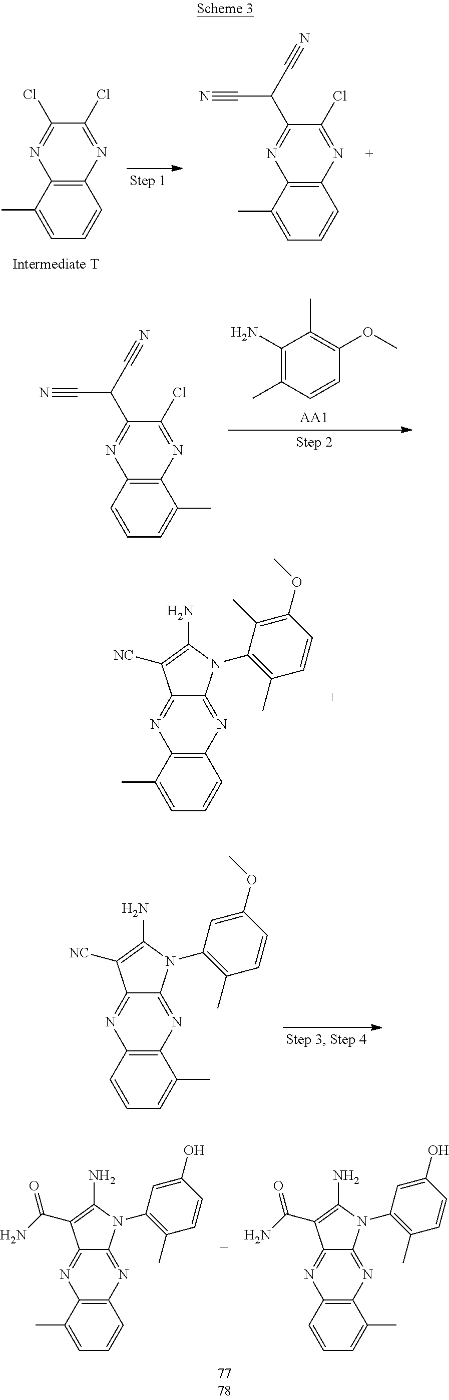

Step 1. Malononitrile (4.1 g, 62.1 mmol) was added portionwise to a vigorously stirred suspension of sodium hydride (60% dispersion in mineral oil, 2.47 g, 64.4 mmol) in DME (100 mL). After the addition, the stirring was continued for 30 min and then 2,3-dichloro-5-methyl-quinoxaline (intermediate T, 6.5 g, 30.5 mmol) was added. The reaction mixture was stirred at rt for 15 min and then heated under reflux for 4 h. The DME was evaporated and cold aqueous 1M HCl was added to give a yellow precipitate that was filtered and washed with water to afford a mixture of 2-(3-chloro-5-methyl-quinoxalin-2-yl)propanedinitrile and 2-(3-chloro-8-methyl-quinoxalin-2-yl)propanedinitrile (5.8 g, 78% yield) (in about 1:1 ratio estimated by UPLCMS) as a yellow solid. MS: [M−1]: 241.1.

Step 2. 3-methoxy-2,6-dimethyl-aniline (arylamine AA1, 1.9 g, 12.5 mmol) was added to a mixture of 2-(3-chloro-5-methyl-quinoxalin-2-yl)propanedinitrile and 2-(3-chloro-8-methyl-quinoxalin-2-yl)propanedinitrile (1.00 g, 4.12 mmol) in NMP (10 mL). The reaction mixture was heated to 130° C. for 6 h, cooled, and the mixture was poured into aqueous NaHCO₃ sat. The precipitate was collected by filtration, washed with water and dried by codistillation with toluene twice. The brown residue was adsorbed on silica using 15% MeOH in DCM and purified by flash chromatography on silica gel (20-60% EtOAc in hexanes to provide a mixture of 2-amino-1-(3-methoxy-2,6-dimethyl-phenyl)-8-methyl-pyrrolo[3,2-b]quinoxaline-3-carbonitrile and 2-amino-1-(3-methoxy-2,6-dimethyl-phenyl)-5-methyl-pyrrolo[2,3-b]quinoxaline-3-carbonitrile (1.04 g, 71% yield) as a brown solid. MS: [M+1]: 358.2.

Step 3. Following general procedure 1, treatment of the mixture of 2-amino-1-(3-methoxy-2,6-dimethyl-phenyl)-8-methyl-pyrrolo[3,2-b]quinoxaline-3-carbonitrile and 2-amino-1-(3-methoxy-2,6-dimethyl-phenyl)-5-methyl-pyrrolo[2,3-b]quinoxaline-3-carbonitrile (1.04 g, 2.90 mmol) with H₂SO₄ led to a mixture of 2-amino-1-(3-methoxy-2,6-dimethyl-phenyl)-8-methyl-pyrrolo[3,2-b]quinoxaline-3-carboxamide and 2-amino-1-(3-methoxy-2,6-dimethyl-phenyl)-5-methyl-pyrrolo[2,3-b]quinoxaline-3-carboxamide (960 mg, 89% yield) as a yellow solid. MS: [M+1]: 376.2.

Step 4. Deprotection of the mixture of 2-amino-1-(3-methoxy-2,6-dimethyl-phenyl)-8-methyl-pyrrolo[3,2-b]quinoxaline-3-carboxamide and 2-amino-1-(3-methoxy-2,6-dimethyl-phenyl)-5-methyl-pyrrolo[2,3-b]quinoxaline-3-carboxamide (600 mg, 1.60 mmol) with BBr₃ following general procedure 2 provided crude product which was purified by flash chromatography on silica gel (dry load, 0-10% MeOH in DCM) to obtain a mixture of the desired products as an orange solid. Separation of the regioisomers was achieved by preparative HPLC (30-80% MeCN in H₂O, 0.1% formic acid modifier). The recovered tubes were combined and lyophilized, providing 2 regioisomers. The first to elute, (P1, Compound 77, 20 mg, 7% yield) as a pale yellow solid. ¹H NMR (400 MHz, DMSO-d6) δ 9.86-9.36 (m, 1H), 7.93 (s, 2H), 7.76 (d, J=3.4 Hz, 1H), 7.57 (ddd, J=8.2, 1.5, 0.7 Hz, 1H), 7.42 (ddd, J=7.1, 1.6, 0.9 Hz, 1H), 7.35 (d, J=3.4 Hz, 1H), 7.31 (dd, J=8.3, 7.0 Hz, 1H), 7.08 (dt, J=8.3, 0.8 Hz, 1H), 6.94 (d, J=8.3 Hz, 1H), 2.69 (s, 3H), 1.84-1.75 (m, 3H), 1.71 (s, 3H). MS: [M+1]: 362.2. The last to elute (P2, Compound 78, 9 mg, 3% yield) as a yellow solid. ¹H NMR (400 MHz, DMSO-d6) δ 9.60 (s, 1H), 8.12 (s, 1H), 7.89 (s, 2H), 7.77-7.66 (m, 2H), 7.42 (dd, J=8.3, 7.0 Hz, 1H), 7.36-7.26 (m, 2H), 7.09 (dt, J=8.3, 0.7 Hz, 1H), 6.93 (d, J=8.3 Hz, 1H), 2.42 (d, J=0.8 Hz, 3H), 1.81 (d, J=0.7 Hz, 3H), 1.74 (s, 3H). MS: [M+1]: 362.2. Compound 77 is one of the regioisomers 2-amino-1-(3-hydroxy-2,6-dimethyl-phenyl)-5-methyl-pyrrolo[2,3-b]quinoxaline-3-carboxamide and 2-amino-1-(3-hydroxy-2,6-dimethyl-phenyl)-8-methyl-pyrrolo[3,2-b]quinoxaline-3-carboxamide, and compound 78 is the other of the regioisomers 2-amino-1-(3-hydroxy-2,6-dimethyl-phenyl)-5-methyl-pyrrolo[2,3-b]quinoxaline-3-carboxamide and 2-amino-1-(3-hydroxy-2,6-dimethyl-phenyl)-8-methyl-pyrrolo[3,2-b]quinoxaline-3-carboxamide.

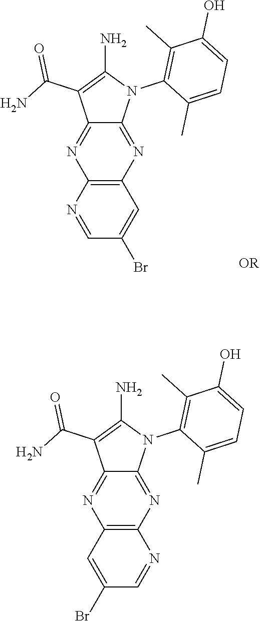 [SMILES: Cc1ccc(O)c(C)c1-n1c(N)c(C(N)=O)c2nc3ncc(Br)cc3nc21 ; Cc1ccc(O)c(C)c1-n1c(N)c(C(N)=O)c2nc3cc(Br)cnc3nc21]

### Compound 79 (2-amino-7-bromo-1-(3-hydroxy-2,6-dimethylphenyl)-1H-pyrido[2,3-b]pyrrolo[2,3-e]pyrazine-3-carboxamide or 2-amino-6-bromo-1-(3-hydroxy-2,6-dimethylphenyl)-1H-pyrido[2,3-b]pyrrolo[3,2-e]pyrazine-3-carboxamide)

Step 1. Malononitrile (4.75 g, 71.9 mmol) was added to a stirred suspension of sodium hydride (60% dispersion in mineral oil, 2.85 g, 74.4) in DME (300 mL). After the addition, the stirring was continued for 30 min, then 7-bromo-2,3-dichloro-pyrido[2,3-b]pyrazine (intermediate W, 10 g, 35.9 mmol) was added. The reaction mixture was stirred at room temperature for 10 min and then refluxed for 3 h. The DME was evaporated and the resulting residue was treated with cold aqueous hydrochloric acid to give a brown solid which was collected by filtration, adsorbed on silica using 10% MeOH in DCM and purified by flash chromatography on silica gel (0-30% MeOH in DCM), affording a brown solid (6.8 g, 61% yield). The product consists of a single regioisomer that is 2-(7-bromo-2-chloro-pyrido[2,3-b]pyrazin-3-yl)propanedinitrile or 2-(7-bromo-3-chloro-pyrido[2,3-b]pyrazin-2-yl)propanedinitrile. MS: [M−1]: 307.9.

Step 2. A mixture of 3-methoxy-2,6-dimethyl-aniline (arylamine AA1, 735 mg, 4.86 mmol) and 2-(7-bromo-2-chloro-pyrido[2,3-b]pyrazin-3-yl)propanedinitrile or 2-(7-bromo-3-chloro-pyrido[2,3-b]pyrazin-2-yl)propanedinitrile (500 mg, 1.62 mmol) in NMP (5 mL) was heated to 130° C. for 6 h, cooled, and the mixture was poured into saturated aqueous NaHCO₃. The precipitate was collected by filtration, washed with water and dried by codistillation with toluene twice. The brown residue was adsorbed on silica using 15% MeOH in DCM and purified by flash chromatography on silica gel (20-100% EtOAc in hexanes), affording a yellow solid (330 mg, 48% yield). The product consists of a single regioisomer that is 2-amino-7-bromo-1-(3-methoxy-2,6-dimethylphenyl)-1H-pyrido[2,3-b]pyrrolo[2,3-e]pyrazine-3-carbonitrile or 2-amino-6-bromo-1-(3-methoxy-2,6-dimethylphenyl)-1H-pyrido[2,3-b]pyrrolo[3,2-e]pyrazine-3-carbonitrile. MS: [M+1]: 425.1.

Step 3. Following general procedure 1, treatment of 2-amino-7-bromo-1-(3-methoxy-2,6-dimethylphenyl)-1H-pyrido[2,3-b]pyrrolo[2,3-e]pyrazine-3-carbonitrile or 2-amino-6-bromo-1-(3-methoxy-2,6-dimethylphenyl)-1H-pyrido[2,3-b]pyrrolo[3,2-e]pyrazine-3-carbonitrile (330 mg, 0.780 mmol) with H₂SO₄ led to a crude product (330 mg, 95% yield) as a yellow solid. The product consists of a single regioisomer that is 2-amino-7-bromo-1-(3-methoxy-2,6-dimethylphenyl)-1H-pyrido[2,3-b]pyrrolo[2,3-e]pyrazine-3-carboxamide or 2-amino-6-bromo-1-(3-methoxy-2,6-dimethylphenyl)-1H-pyrido[2,3-b]pyrrolo[3,2-e]pyrazine-3-carboxamide. MS: [M+1]: 441.1.

Step 4. Deprotection of 2-amino-7-bromo-1-(3-methoxy-2,6-dimethylphenyl)-1H-pyrido[2,3-b]pyrrolo[2,3-e]pyrazine-3-carboxamide or 2-amino-6-bromo-1-(3-methoxy-2,6-dimethylphenyl)-1H-pyrido[2,3-b]pyrrolo[3,2-e]pyrazine-3-carboxamide (330 mg, 0.748 mmol) with BBr₃ following general procedure 2 provided a crude product which was purified by flash chromatography on silica gel (dry load, 0-10% MeOH in DCM) then by preparative HPLC (25-70% MeCN in H₂O, 0.1% formic acid modifier). The recovered tubes were combined and lyophilized to provide a product (Compound 79, 24 mg, 7% yield). The product is a single regioisomer that is either 2-amino-7-bromo-1-(3-hydroxy-2,6-dimethylphenyl)-1H-pyrido[2,3-b]pyrrolo[2,3-e]pyrazine-3-carboxamide or 2-amino-6-bromo-1-(3-hydroxy-2,6-dimethylphenyl)-1H-pyrido[2,3-b]pyrrolo[3,2-e]pyrazine-3-carboxamide. ¹H NMR (400 MHz, DMSO-d6) δ 9.60 (s, 1H), 8.78 (d, J=2.4 Hz, 1H), 8.45 (d, J=2.3 Hz, 1H), 8.30 (s, 2H), 7.75-7.57 (m, 1H), 7.44 (s, 1H), 7.05 (d, J=8.3 Hz, 1H), 6.92 (d, J=8.3 Hz, 1H), 1.77 (s, 3H), 1.69 (s, 3H). MS: [M+1]: 429.0.

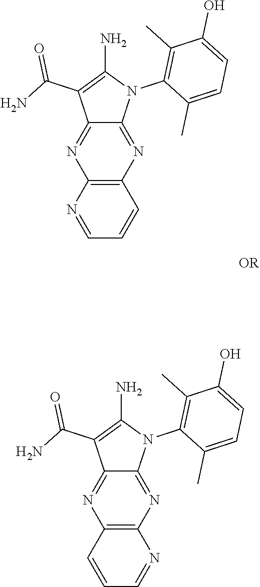 [SMILES: Cc1ccc(O)c(C)c1-n1c(N)c(C(N)=O)c2nc3ncccc3nc21 ; Cc1ccc(O)c(C)c1-n1c(N)c(C(N)=O)c2nc3cccnc3nc21]

### Compound 82 (2-amino-1-(3-hydroxy-2,6-dimethylphenyl)-1H-pyrido[2,3-b]pyrrolo[2,3-e]pyrazine-3-carboxamide or 2-amino-1-(3-hydroxy-2,6-dimethylphenyl)-1H-pyrido[2,3-b]pyrrolo[3,2-e]pyrazine-3-carboxamide)

A mixture of Compound 79 (100 mg, 0.234 mmol) and palladium on carbon (10% w/w, 25 mg) in EtOH (5 mL) was stirred under H₂ atmosphere (balloon) for 2 h. The reaction mixture was filtrated on a 0.45 micron PTFE filter and the filtrate was purified by prep HPLC (25-70% MeCN in H₂O, 0.1% formic acid modifier). The recovered tubes were combined and lyophilized to provide a pale yellow solid (Compound 82, 41 mg, 50% yield). The product consists of a single regioisomer that is 2-amino-1-(3-hydroxy-2,6-dimethylphenyl)-1H-pyrido[2,3-b]pyrrolo[2,3-e]pyrazine-3-carboxamide or 2-amino-1-(3-hydroxy-2,6-dimethylphenyl)-1H-pyrido[2,3-b]pyrrolo[3,2-e]pyrazine-3-carboxamide. ¹H NMR (400 MHz, DMSO-d6) δ 9.65 (s, 1H), 9.09 (s, 1H), 8.96-8.84 (m, 2H), 8.82 (dd, J=8.2, 1.5 Hz, 1H), 7.78 (dd, J=8.2, 5.7 Hz, 2H), 7.48 (s, 1H), 7.07 (d, J=8.3 Hz, 1H), 6.96 (d, J=8.3 Hz, 1H), 1.79 (s, 3H), 1.72 (s, 3H). MS: [M+1]: 349.2.

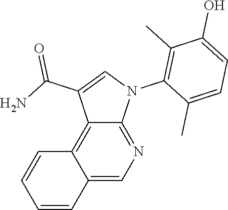 [SMILES: Cc1ccc(O)c(C)c1-n1cc(C(N)=O)c2c3ccccc3cnc21]

### Compound 88 (3-(3-hydroxy-2,6-dimethylphenyl)-3H-pyrrolo[2,3-c]isoquinoline-1-carboxamide)

Step 1. To a solution of intermediate P (410 mg, 1.81 mmol) and arylamine AA1 (302 mg, 2.00 mmol) in THE (6.5 mL) was added dropwise a solution of LiHMDS in THE (1M, 3.81 mL, 3.81 mmol) at RT under nitrogen. The reaction mixture was stirred at 65° C. for 1 h. The reaction was cooled to RT and partitioned between EtOAc and sat aq NH₄Cl. The organic phase was washed with brine, dried over Na₂SO₄ and evaporated. The residue was purified by silica gel chromatography using EtOAc in heptanes (0 to 20%), and then by reverse phase flash chromatography (C18) using MeOH in H₂O (25 to 100%) to get 4-bromo-N-(3-methoxy-2,6-dimethylphenyl)isoquinolin-3-amine (418.0 mg, 64% yield) as a beige foam. MS: [M+1]: 357.0, 359.0.

Step 2. In a sealed tube, to a solution of malononitrile (155 mg, 2.35 mmol) in 1,2-dimethoxyethane (5 mL) was added sodium tert-butoxide (215 mg, 2.24 mmol) and the resulting solution was stirred for 20 min then, 4-bromo-N-(3-methoxy-2,6-dimethylphenyl)isoquinolin-3-amine (400 mg, 1.12 mmol) and Pd(dppf)Cl₂.DCM (69 mg, 0.084 mmol) were added. The reaction mixture was stirred at 105° C. for 22 hours. The resulting suspension was cooled to RT and filtered through a plug of silica gel, eluted with EtOAc. The filtrate was washed with water/brine and brine, dried over Na₂SO₄ and evaporated. The residue was purified by silica gel chromatography using EtOAc in heptanes (5 to 50%) to get 2-amino-3-(3-methoxy-2,6-dimethylphenyl)-3H-pyrrolo[2,3-c]isoquinoline-1-carbonitrile (297 mg, 77% yield) as a yellow solid. MS: [M+1]: 343.2.

Step 3. Following the procedure described for compound 24 step 1 using 2-amino-3-(3-methoxy-2,6-dimethylphenyl)-3H-pyrrolo[2,3-c]isoquinoline-1-carbonitrile (200 mg, 0.584 mmol) as starting material, 3-(3-methoxy-2,6-dimethylphenyl)-3H-pyrrolo[2,3-c]isoquinoline-1-carbonitrile (154 mg, 80% yield) was obtained as a beige solid after purification by silica gel chromatography using EtOAc in heptanes (0 to 50%), and then by reverse phase flash chromatography (C18) using MeOH in H₂O (15 to 100%). MS: [M+1]: 328.2.

Step 4. To a solution of 3-(3-methoxy-2,6-dimethylphenyl)-3H-pyrrolo[2,3-c]isoquinoline-1-carbonitrile (150 mg, 0.458 mmol) in ethanol (3 mL) and water (250 μL) was added Ghaffar-Parkins catalyst (7.0 mg, 0.016 mmol). The reaction mixture was heated to 80° C. to get a solution. After 23 hours of stirring, the reaction mixture was poured into EtOAc and washed with water and brine, dried over Na₂SO₄ and evaporated to get 3-(3-methoxy-2,6-dimethylphenyl)-3H-pyrrolo[2,3-c]isoquinoline-1-carboxamide (164 mg, quantitative) as a yellow solid. MS: [M+1]: 346.2.

Step 5. Following general procedure 2, deprotection of 3-(3-methoxy-2,6-dimethylphenyl)-3H-pyrrolo[2,3-c]isoquinoline-1-carboxamide (156 mg, 0.452 mmol) afforded 3-(3-hydroxy-2,6-dimethylphenyl)-3H-pyrrolo[2,3-c]isoquinoline-1-carboxamide (compound 88, 100 mg, 66% yield) as a light brown fluffy solid after purification by reverse phase flash chromatography on C18 cartridge (15-100% MeOH in H₂O). ¹H NMR (400 MHz, DMSO-d6) δ ppm 1.67 (s, 3H), 1.78 (s, 3H), 6.94 (d, J=8.3 Hz, 1H), 7.08 (d, J=8.3 Hz, 1H), 7.12-7.27 (m, 1H), 7.59 (ddd, J=8.0, 6.9, 1.0 Hz, 1H), 7.73 (br. s, 1H), 7.81 (ddd, J=8.5, 7.0, 1.3 Hz, 1H), 8.09 (s, 1H), 8.16 (d, J=8.1 Hz, 1H), 8.92 (s, 1H), 9.56 (s, 1H), 9.65 (d, J=8.6 Hz, 1H). MS: [M+1]: 332.1.

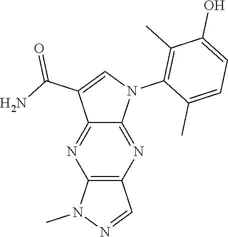 [SMILES: Cc1ccc(O)c(C)c1-n1cc(C(N)=O)c2nc3c(cnn3C)nc21]

### Compound 89 (5-(3-hydroxy-2,6-dimethylphenyl)-1-methyl-1,5-dihydropyrazolo[4,3-b]pyrrolo[3,2-e]pyridine-7-carboxamide)

Step 1. A vial was charged with arylamine AA1 (240 mg, 1.59 mmol), intermediate Q (420 mg, 1.44 mmol), Xantphos (63 mg, 0.11 mmol), cesium carbonate (1.00 g, 3.07 mmol) and 1,2-dimethoxyethane (5 mL). The suspension was sparged with nitrogen over the sonic bath for 5 min and Pd₂(dba)₃ (100 mg, 0.109 mmol) was added. The reaction mixture was heated to 82° C. for 16 h. The suspension was cooled to RT and filtered through a small pad of silica gel, rinsed with EtOAc. The filtrate was evaporated, and the crude product was purified by silica gel chromatography using EtOAc in heptanes (0 to 40%) to provide 6-bromo-N-(3-methoxy-2,6-dimethylphenyl)-1-methyl-1H-pyrazolo[4,3-b]pyridin-5-amine (280 mg, 53% yield) as an off-white solid. MS: [M+1]: 361.0, 363.0

Step 2. Following the procedure described for compound 88 step 2 using 6-bromo-N-(3-methoxy-2,6-dimethylphenyl)-1-methyl-1H-pyrazolo[4,3-b]pyridin-5-amine (240 mg, 0.664 mmol) as starting material, 6-amino-5-(3-methoxy-2,6-dimethylphenyl)-1-methyl-1,5-dihydropyrazolo[4,3-b]pyrrolo[3,2-e]pyridine-7-carbonitrile (141 mg, 61% yield) was obtained as an orange solid after purification by silica gel chromatography using EtOAc in heptanes (25 to 100%). MS: [M+1]: 347.2.

Step 3. Following the procedure described for compound 24 step 1 using 6-amino-5-(3-methoxy-2,6-dimethylphenyl)-1-methyl-1,5-dihydropyrazolo[4,3-b]pyrrolo[3,2-e]pyridine-7-carbonitrile (141 mg, 0.407 mmol) as starting material, 5-(3-methoxy-2,6-dimethylphenyl)-1-methyl-1,5-dihydropyrazolo[4,3-b]pyrrolo[3,2-e]pyridine-7-carbonitrile (68 mg, 50% yield) was obtained as a yellow foam after purification by silica gel chromatography using EtOAc in heptanes (20 to 90%). MS: [M+1]: 332.2.

Step 4. Following the procedure described for compound 88 step 4 using 5-(3-methoxy-2,6-dimethylphenyl)-1-methyl-1,5-dihydropyrazolo[4,3-b]pyrrolo[3,2-e]pyridine-7-carbonitrile (67 mg, 0.20 mmol) as starting material, 5-(3-methoxy-2,6-dimethylphenyl)-1-methyl-1,5-dihydropyrazolo[4,3-b]pyrrolo[3,2-e]pyridine-7-carboxamide (70 mg, 99% yield) was obtained as a red oil. MS: [M+1]: 350.2.

Step 5. Following general procedure 2, deprotection of 5-(3-methoxy-2,6-dimethylphenyl)-1-methyl-1,5-dihydropyrazolo[4,3-b]pyrrolo[3,2-e]pyridine-7-carboxamide (70 mg, 0.20 mmol) afforded 5-(3-hydroxy-2,6-dimethylphenyl)-1-methyl-1,5-dihydropyrazolo[4,3-b]pyrrolo[3,2-e]pyridine-7-carboxamide (compound 89, 31 mg, 46% yield) as white fluffy solid after purification by reverse phase flash chromatography on 018 cartridge (10-100% MeOH in H₂O). ¹H NMR (400 MHz, DMSO-d6) b ppm 1.67 (s, 3H), 1.77 (s, 3H), 4.15 (s, 3H), 6.93 (d, J=8.3 Hz, 1H), 7.07 (d, J=8.3 Hz, 1H), 7.21 (br. s, 1H), 7.55 (br. s, 1H), 8.13 (d, J=1.0 Hz, 1H), 8.34 (s, 1H), 8.77 (d, J=0.7 Hz, 1H), 9.56 (s, 1H). MS: [M+1]: 336.3.

### Examples of Arylamines Preparation

A variety of arylamines were used to prepare compounds of the present invention. Some of these arylamines were commercially available and some were prepared. Table 2 lists some examples of such arylamines for which the preparation is described herein.

TABLE 2

| | |
|---|---|
| 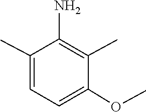 | Arylamine AA1 |
| 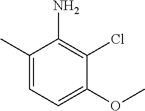 | Arylamine AA5 |
| 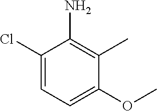 | Arylamine AA6 |
| 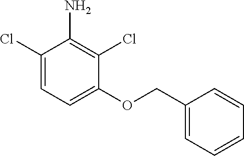 [SMILES: Nc1c(Cl)ccc(OCc2ccccc2)c1Cl] | Arylamine AA7 |
| 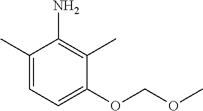 | Arylamine AA8 |
| 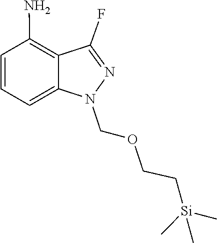 [SMILES: C[Si](C)(C)CCOCn1nc(F)c2c(N)cccc21] | Arylamine AA9 |
| 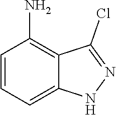 [SMILES: Nc1cccc2[nH]nc(Cl)c12] | Arylamine AA10 |
| 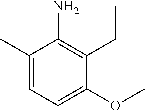 | Arylamine AA11 |
| 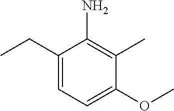 | Arylamine AA12 |
| 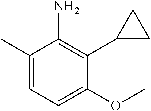 | Arylamine AA13 |
| 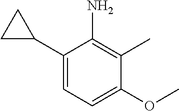 | Arylamine AA14 |

### Preparation of Arylamine AA1

Compounds of the present invention can be prepared from arylamine AA1 which can be prepared as shown in Scheme AA1 and described herein. Commercially available 1,3-dimethyl-2-nitrobenzene can be brominated under suitable bromination conditions. The resulting bromo can be converted to a methoxy upon treatment with sodium methoxide and copper(I) bromide. The nitro can be reduced to generate arylamine AA1.

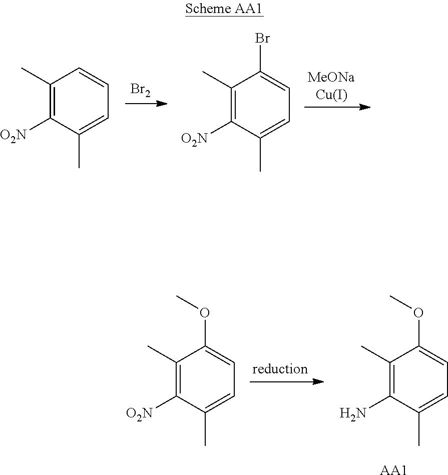

Step 1. A 3 necked 3 L round-bottom flask was equipped with a mechanical stirrer, reflux condenser and addition funnel and loaded with 1,3-dimethyl-2-nitro-benzene (300 g, 1.98 mol), DCM (900 mL), iron powder (28.0 g, 501 mmol) and iron(III) bromide (11.9 g, 40.3 mmol). Bromine (112 mL, 2.19 mol) was added dropwise via an addition funnel over 45-60 min. Internal monitoring of the temperature showed an exotherm to 30° C. 90 min after the addition of bromine was complete, more bromine (5 mL, 98 mmol) was added and the reaction mixture was stirred for another 45 min to complete conversion. The reaction mixture was diluted with ice water (1.5 L) and Et₂O (1.5 L). The layers were separated. The aqueous layer was back extracted with Et₂O (0.5 L). The combined organic layers were washed with aqueous 20% Na₂S₂O₃ (1 L), brine (500 mL), dried over Na₂SO₄, filtered over a silica gel pad (300 cc), concentrated then dried in vacuo to provide 1-bromo-2,4-dimethyl-3-nitro-benzene (451.5 g, 99% yield) as an off-white solid.

Step 2. A 5 L 4 neck round bottom flask equipped with a mechanical stirrer and a reflux condenser was charged with 1-bromo-2,4-dimethyl-3-nitro-benzene (451.5 g, 1.96 mol) in DMF (1.6 L). CuBr (28.0 g, 195 mmol) was added followed by sodium methoxide (1.31 L, 5.89 mol, 25% in MeOH). The reaction mixture was slowly heated to 95° C., achieving a mild reflux. After 6 h, the reaction mixture was left to cool to rt overnight. The reaction mixture was diluted with Et₂O and saturated aqueous NH₄Cl (1.5 L each). The layers were separated, and the aqueous layer was back extracted with Et₂O (750 mL). The combined organic extracts were washed with brine (750 mL), dried over Na₂SO₄ and filtered over a silica pad, rinsed with Et₂O and concentrated, dried in vacuo to provide 1-methoxy-2,4-dimethyl-3-nitro-benzene (352 g, 99% yield) as an ochre solid.

Step 3. To a solution of 1-methoxy-2,4-dimethyl-3-nitro-benzene (115 g, 635 mmol) in EtOH (1.5 L) in a 3 neck 3 L flask equipped with a mechanical stirrer was added iron powder (213 g, 3.81 mol), then a solution of ammonium chloride (204 g, 3.81 mol) in water (500 mL) was added portion wise. The mixture was heated to 85° C. for 8 h. The mixture was cooled down to rt and filtered on Celite. The volume of the filtrate was reduced (most of the EtOH was evaporated) and the resulting mixture was diluted with Et₂O (800 mL) and water (150 mL). The layers were separated, and the aqueous layer was back extracted with Et₂O (500 mL). The combined organic extracts were washed with brine, dried over Na₂SO₄, filtered, concentrated and dried in vacuo to provide 3-methoxy-2,6-dimethyl-aniline (89.1 g, 93% yield) as a brown oil. ¹H NMR (400 MHz, Chloroform-d) δ 6.88 (dq, J=8.3, 0.7 Hz, 1H), 6.31 (d, J=8.2 Hz, 1H), 3.79 (s, 3H), 3.61 (brs, 2H), 2.14 (d, J=0.7 Hz, 3H), 2.07 (s, 3H). MS: [M+1]: 152.3.

### Preparation of Arylamine AA5

Compounds of the present invention can be prepared from arylamine AA5 which can be prepared as shown in Scheme AA5 and described herein. Commercially available 2-chloro-3-methoxy-benzoic acid can be brominated with a suitable bromination reagent and the carboxylic acid can be converted to a NHBoc under Curtius conditions. The bromo can be converted to a methyl and the NHBoc can be cleaved under acidic conditions to generate arylamine AA5.

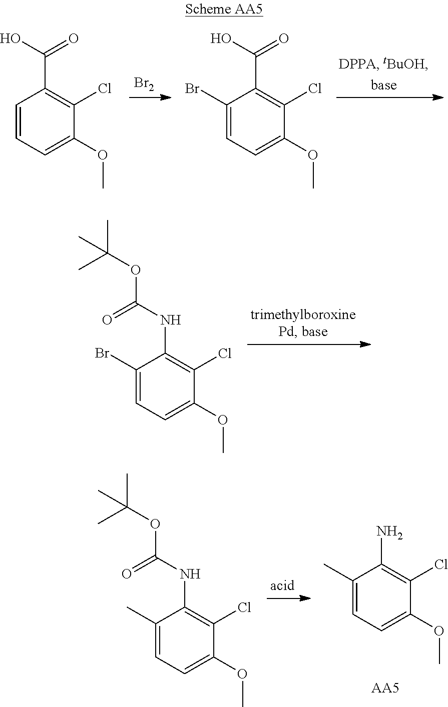

Step 1. To a solution of 2-chloro-3-methoxy-benzoic acid (50 g, 268 mmol) in AcOH (250 mL) and water (250 mL) was added bromine (27.5 mL, 537 mmol) dropwise. The mixture was stirred at 60° C. for 18 h, cooled to rt, brine was added, and the mixture was extracted twice with DCM. The combined organic extracts were dried over Na₂SO₄, filtered and concentrated in vacuo to provide 6-bromo-2-chloro-3-methoxy-benzoic acid (71 g, quantitative yield) as a brown oil which solidified upon standing under vacuum over the weekend.

Step 2. To a solution of 6-bromo-2-chloro-3-methoxy-benzoic acid (23.6 g, 88.9 mmol). Et₃N (38 mL, 271 mmol) and tert-butanol (42.5 mL, 450 mmol) in toluene (500 mL) was added [azido(phenoxy)phosphoryl]oxybenzene (29.5 mL, 136 mmol). The mixture was heated at 100° C. for 16 h, cooled down to rt then the volatiles were removed in vacuo. The residue was diluted with EtOAc (100 mL), and the organic layer was washed with 5% citric acid, water, saturated aqueous NaHCO₃, brine, dried over Na₂SO₄, filtered and concentrated to dryness. The residue was purified by silica gel chromatography eluting with a gradient of 0 to 20% EtOAc in hexanes to provide tert-butyl N-(6-bromo-2-chloro-3-methoxy-phenyl)carbamate (16.2 g, 54% yield) as a yellowish solid.

Step 3. To a solution of tert-butyl N-(6-bromo-2-chloro-3-methoxy-phenyl)carbamate (25 g, 74.3 mmol) in dioxane (500 mL) was added trimethylboroxine (50% w/w in THF, 20.51 g, 81.7 mmol). PdCl₂(dppf.CH₂Cl₂ (5.22 g, 7.43 mmol) and aqueous Na₂CO₃ (2 M, 111 mL, 223 mmol). The mixture was heated at 100° C. for 16 h, cooled down to rt then volatiles were removed in vacuo. EtOAc and water were added. The organic layer was separated and washed with brine, dried over Na₂SO₄, filtered and concentrated to dryness. The residue was purified by silica gel chromatography eluting with a gradient of 0 to 30% EtOAc in heptane to provide tert-butyl N-(2-chloro-3-methoxy-6-methyl-phenyl)carbamate (13.8 g, 68% yield) as a yellowish solid.

Step 4. HCl in dioxane (4 M, 100 mL) was added to a solution of tert-butyl N-(2-chloro-3-methoxy-6-methyl-phenyl)carbamate (13.8 g, 50.8 mmol) in MeOH (100 mL). After 3 h, the volatiles were evaporated to dryness under vacuum to provide a white solid to which was added under vigorous stirring 250 mL of EtOAc and 250 mL of aqueous saturated NaHCO₃. The organic layer was separated. The aqueous layer was back extracted with EtOAc. The combined organic layers were washed with brine, dried over Na₂SO₄, filtered and concentrated in vacuo. The residue was purified by silica gel chromatography eluting with a gradient of 0 to 30% EtOAc in heptane to provide 2-chloro-3-methoxy-6-methyl-aniline (7.9 g, 91% yield) as a clear oil which solidified upon standing. ¹H NMR (400 MHz, Chloroform-d) δ 6.95-6.79 (m, 1H), 6.27 (dd, J=8.3, 1.5 Hz, 1H), 4.06 (br s, 2H), 3.83 (d, J=1.6 Hz, 3H), 2.12 (d, J=0.8 Hz, 3H). MS: [M+1]: 172.2.

### Preparation of Arylamine AA6

Compounds of the present invention can be prepared from arylamine AA6 which can be prepared as shown in Scheme AA6 and described herein. Commercially available 3-methoxy-2-methyl-aniline can be chlorinated with a chlorination reagent to generate arylamine AA6.

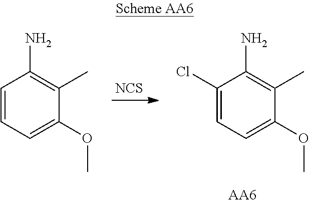

Step 1. NCS (98 g, 734 mmol) was added in 4 portions (15 minutes between each addition) to a solution of 3-methoxy-2-methyl-aniline (100 g, 729 mmol) in DCM (500 mL) at 0° C. 30 min after the last addition, 100 g of silica gel was added, the mixture was evaporated under vacuum and the black residue was purified by silica gel chromatography (dry load) in eluting with a gradient of 0 to 10% EtOAc in hexanes to provide 6-chloro-3-methoxy-2-methyl-aniline (55.6 g, 44% yield) as an orange solid. ¹H NMR (400 MHz, Chloroform-d) δ 7.08 (d, J=8.8 Hz, 1H), 6.28 (d, J=8.8 Hz, 1H), 4.02 (br s, 2H), 3.78 (s, 3H), 2.07 (s, 3H). MS: [M+1]: 172.3.

### Preparation of Arylamine AA7

Compounds of the present invention can be prepared from arylamine AA7 which can be prepared as shown in Scheme AA7 and described herein. Commercially available 3-amino-2,4-dichloro-phenol can be O-protected with a suitable protecting group such as O-PMB to generate arylamine AA7.

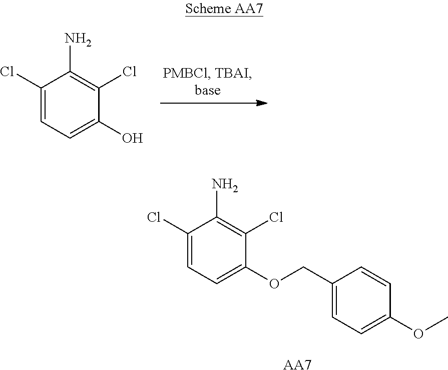

To a suspension of 3-amino-2,4-dichloro-phenol.HCl salt (20 g, 93.3 mmol) in DMF (150 mL) was added 1-(chloromethyl)-4-methoxy-benzene (14.0 mL, 103 mmol), tetrabutylammonium iodide (1 g, 3.00 mmol) and Cs₂CO₃ (64.0 g, 196 mmol). The mixture was stirred at 40° C. overnight, then it was diluted with water, stirred for 20 min, and filtered. The precipitate was washed with water and dried in vacuo. The resulting crude product was purified by silica gel chromatography eluting with a gradient of 0 to 100% DCM in hexanes to provide 2,6-dichloro-3-[(4-methoxyphenyl)methoxy]aniline (20 g, 72% yield) as an off-white solid. ¹H NMR (400 MHz, Chloroform-d) δ 7.38-7.30 (m, 2H), 7.05 (d, J=8.9 Hz, 1H), 6.92-6.77 (m, 2H), 6.32 (s, 1H), 5.01 (s, 2H), 4.46 (s, 2H), 3.79 (s, 3H). MS: [M+1]: 298.0.

### Preparation of Arylamine AA8

Compounds of the present invention can be prepared from arylamine AA8 which can be prepared as shown in Scheme AA8 and described herein. The commercially available 4-methyl-3-nitro-phenol can be O-protected with a suitable protecting group such as O-MOM. The nitro can be reduced to generate arylamine AA8.

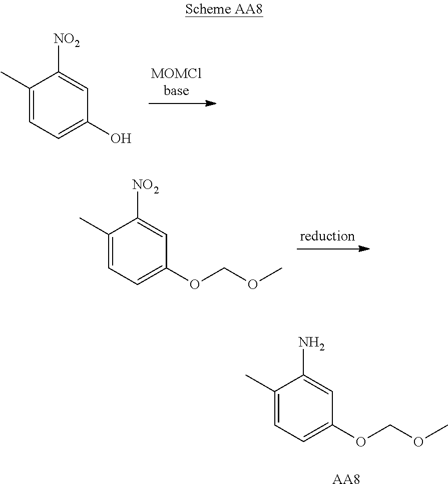

Step 1. To a suspension of 4-methyl-3-nitro-phenol (25 g, 163 mmol) in DCM (250 mL) was added DIPEA (34 mL, 195 mmol) followed by chloro(methoxy)methane (26.0 g, 323 mmol, 24.5 mL) added dropwise. After stirring 18 h, the reaction mixture was washed with water. The layers were separated. The organic layer was washed with 0.2N HCl (2×), brine, dried over MgSO₄, filtered and concentrated, then dried in vacuo affording 4-(methoxymethoxy)-1-methyl-2-nitro-benzene (31.4 g, 98% yield) as a dark red oil.

Step 2. To a suspension of 4-(methoxymethoxy)-1-methyl-2-nitro-benzene (31.4 g, 159 mmol) in EtOH (200 mL) and water (75 mL) was added ammonium chloride (43.3 g, 809 mmol) then iron powder (44.5 g, 796 mmol). The reaction mixture was heated to 80° C. for 3.5 h, then temperature was increased to 90° C., stirring for 4 days. The reaction mixture was cooled to rt, filtered and rinsed with EtOAc. The filtrate was concentrated and diluted with EtOAc and saturated aqueous NaHCO₃. The layers were separated, and the aqueous layer was back extracted with EtOAc (2×). The combined organic extracts were washed with brine, dried over MgSO₄, filtered and concentrated, affording 26.3 g crude product as a dark brown oil which was purified on a silica gel pad, eluting with 20-30% EtOAc in hexanes. The pure fractions were combined, concentrated, then dried in vacuo, affording 5-(methoxymethoxy)-2-methyl-aniline (25.4 g, 95% yield) as a purple oil. ¹H NMR (400 MHz, Chloroform-d) δ 6.94 (dd, J=8.0, 1.7 Hz, 1H), 6.45-6.30 (m, 2H), 5.12 (s, 2H), 3.47 (s, 3H), 2.10 (s, 3H). MS: [M+1]: 168.3.

### Preparation of Arylamine AA9

Compounds of the present invention can be prepared from arylamine AA9 which can be prepared as shown in Scheme AA9 and described herein. The commercially available 4-nitro-1H-indazole can be fluorinated with a suitable fluorinating agent. The indazole can be protected with a SEM protecting group and the nitro can be reduced to generate arylamine AA10.

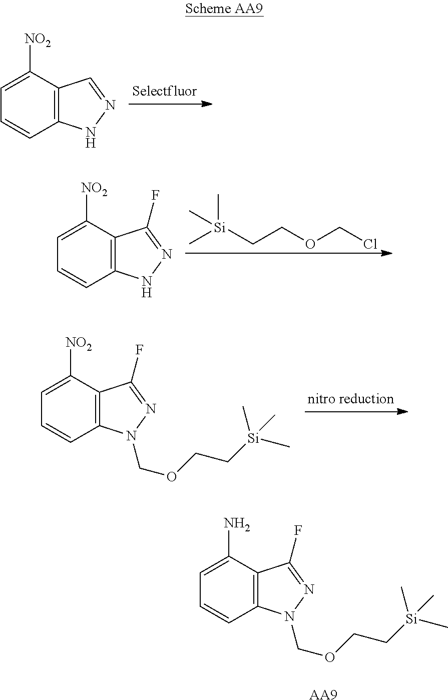

Step 1. To a solution of 4-nitro-1H-indazole (5.0 g, 30.7 mmol) in MeCN (75 mL) was added Selectfluor (21.72 g, 61.30 mmol). The mixture was stirred at 100° C. for 10 h, cooled down to rt and diluted with water and 2M Na₂CO₃ and stirred at rt for 20 min. The solid was collected by filtration, washed with water and dried in vacuo to provide 3-fluoro-4-nitro-1H-indazole (510 mg, 9% yield) as an off-white solid which contained about 10% starting material and was used as such for the next step. ¹H NMR (400 MHz, DMSO-d6) δ 13.43 (s, 1H), 8.07 (d, J=7.7 Hz, 1H), 7.94 (d, J=8.5 Hz, 1H), 7.59 (dd, J=8.5, 7.7 Hz, 1H). MS: [M−1]: 180.0.

Step 2. To a solution of 3-fluoro-4-nitro-1H-indazole (3.46 g, 19.1 mmol) in DMF (15 mL) were added cesium carbonate (7 g, 21 mmol), 2-(2-chloroethoxy)ethyl-trimethyl-silane (4.14 g, 24.8 mmol, 4.4 mL) and tetrabutylammoniumiodide (450 mg, 1.35 mmol). The mixture was stirred at rt for 1 h, then it was diluted with EtOAc, washed with water (2×), followed by brine, dried over sodium sulfate, filtered and the filtrate was concentrated to dryness. The residue was purified by flash chromatography on silica gel (0-35% EtOAc in hexanes) to provide 2-[(3-fluoro-4-nitro-indazol-1-yl)methoxy]ethyl-trimethyl-silane (3.1 g, 52% yield) and in about 10:1 ratio as a brown solid (10:1 ratio with minor regioisomer 2-[(3-fluoro-4-nitro-indazol-2-yl)methoxy]ethyl-trimethyl-silane). ¹H NMR (400 MHz, Chloroform-d) δ 8.10 (m 1H), 7.84 (m, 1H), 7.55 (m, 1H), 5.64 (s, 2H), 3.61-3.47 (m, 2H), 0.93-0.75 (m, 2H), −0.07 (s, 9H).

Step 3. To a solution of 2-[(3-fluoro-4-nitro-indazol-1-yl)methoxy]ethyl-trimethyl-silane (3 g, 9.6 mmol) in MeOH (20 mL) was added palladium on carbon (10% w/w, 600 mg, 0.564 mmol). The mixture was stirred at rt under a hydrogen atmosphere (balloon) for 5 h. Then the mixture was filtered over a pad of celite. The filtrate was concentrated and dried in vacuo to provide 3-fluoro-1-(2-trimethylsilylethoxymethyl)indazol-4-amine (2.5 g, 92% yield) as a brown thick oil. ¹H NMR (400 MHz, Chloroform-d) δ 7.18 (dd, J=8.4, 7.6 Hz, 1H), 6.76 (m, 1H), 6.32-6.24 (m, 1H), 5.46 (s, 2H), 4.31 (s, 2H), 3.61-3.48 (m, 2H), 0.99-0.63 (m, 2H), −0.07 (s, 9H).

### Preparation of Arylamine AA10

Compounds of the present invention can be prepared from arylamine AA10 which can be prepared as shown in Scheme AA10 and described herein. The commercially available 4-nitro-1H-indazole can be chlorinated with a suitable chlorinating agent. The nitro can be reduced to generate arylamine AA10.

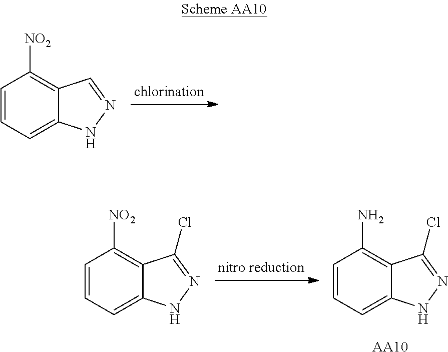

Step 1. To a solution of sodium hydroxide (3.04 g, 76.0 mmol) in H₂O (100 mL) was added 4-nitro-1H-indazole (3.0 g, 18 mmol). The resulting mixture was heated to 50° C. for 35 min, then cooled in an ice bath. Aqueous sodium hypochlorite (0.806 M, 35 mL) was added and the cold bath was removed. After 6 h, more aqueous sodium hypochlorite (0.806 M, 15 mL) was added and the mixture was stirred overnight. It was then cooled in an ice bath, acidified to pH 2 with 3M HCl (about 30 mL). The resulting precipitate was stirred in the ice bath then the solids were collected by filtration and washed with H₂O and air-dried, affording 3-chloro-4-nitro-1H-indazole (3.39 g, 93% yield) as a light tan solid. ¹H NMR (400 MHz, DMSO-d6) δ 8.02 (dd, J=8.5, 0.8 Hz, 1H), 7.99 (dd, J=7.6, 0.8 Hz, 1H), 7.64 (dd, J=8.5, 7.6 Hz, 1H). MS: [M−1]: 196.0.

Step 2. To a RBF equipped with a condenser and charged with 3-chloro-4-nitro-1H-indazole (1.0 g, 5.06 mmol), ammonium chloride (1.35 g, 25.3 mmol), H₂O (5 mL) and EtOH (10 mL) was added iron powder (1.40 g, 25.1 mmol). The reaction mixture was stirred at to 80° C. for 45 min and brought back to RT. The reaction mixture was filtered, the solids were washed with EtOAc. The filtrate was diluted with H₂O, the layers were separated and the aqueous layer extracted with EtOAc (2×). The combined organic extracts were washed with H₂O then brine, dried over Na₂SO₄ and filtered through a silica pad (ca 40 cc), eluting with EtOAc, concentrated and dried in vacuo, providing 3-chloro-1H-indazol-4-amine (757 mg, 89% yield) as a dark olive solid. ¹H NMR (400 MHz, DMSO-d6) δ 12.82 (br s, 1H), 7.05 (dd, J=8.3, 7.5 Hz, 1H), 6.61 (dd, J=8.3, 0.7 Hz, 1H), 6.21 (dd, J=7.6, 0.7 Hz, 1H), 5.58 (br s, 2H). MS: [M+1]: 168.2.

### Examples of 2,3-dichloropyrazine preparation and fused 2,3-dihalopyridines

A variety of 2,3-dichloropyrazine intermediates and fused 2,3-dihalopyridines intermediates were used to prepare compounds of the present invention. Table 3 lists some examples of such 2,3-dichloropyrazine intermediates and fused 2,3-dihalopyridines intermediates for which the preparation is described herein.

TABLE 3

| | |
|---|---|
| 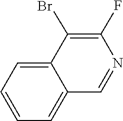 [SMILES: Fc1ncc2ccccc2c1Br] | Intermediate P |
| 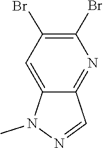 | Intermediate Q |
| 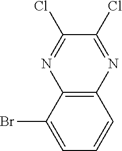 [SMILES: Clc1nc2cccc(Br)c2nc1Cl] | Intermediate R |
| 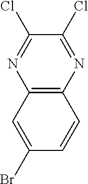 [SMILES: Clc1nc2ccc(Br)cc2nc1Cl] | Intermediate S |
| 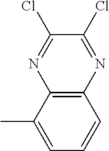 | Intermediate T |
| 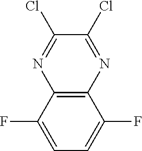 [SMILES: Fc1ccc(F)c2nc(Cl)c(Cl)nc12] | Intermediate U |
| 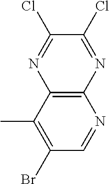 | Intermediate V |
| 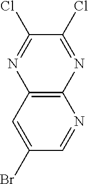 [SMILES: Clc1nc2cc(Br)cnc2nc1Cl] | Intermediate W |
| 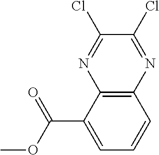 [SMILES: COC(=O)c1cccc2nc(Cl)c(Cl)nc12] | Intermediate X |
| 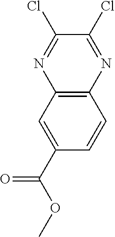 [SMILES: COC(=O)c1ccc2nc(Cl)c(Cl)nc2c1] | Intermediate Y |
| 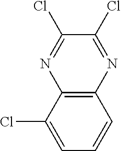 [SMILES: Clc1nc2cccc(Cl)c2nc1Cl] | Intermediate Z |

### Preparation of Intermediate P

Bromination of isoquinolin-3-amine followed by diazotization of the amine and treatment with HF-pyridine gives access to the required 4-bromo-3-fluoroisoquinoline as illustrated in Scheme 5.

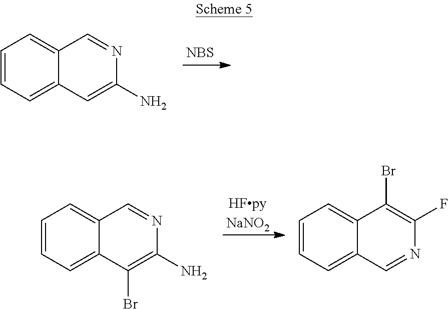

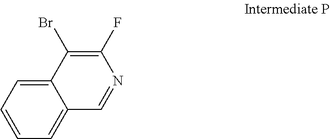 [SMILES: Fc1ncc2ccccc2c1Br]

Step 1. To a solution isoquinolin-3-amine (2.20 g, 15.3 mmol) in dichloromethane (50 mL) and methanol (50 mL) was added portion wise NBS (3.26 g, 18.3 mmol) below 20° C. After 1 h of stirring, the reaction mixture was diluted in 250 mL of DCM and was washed with 5% sodium thiosulfate (aq), water and brine, dried over Na₂SO₄ and evaporated. The crude product was purified by silica gel chromatography using EtOAc in heptanes (0 to 50%) to get 4-bromoisoquinolin-3-amine (1.80 g, 52% yield) as brown solid.

Step 2. To a solution of 4-bromoisoquinolin-3-amine (500 mg, 2.24 mmol) in 70% hydrogen fluoride-pyridine (5 g, 50.5 mmol) was added portion wise sodium nitrite (201 mg, 2.91 mmol) at 0-5° C. After 20 minutes of stirring, the reaction mixture was stirred at room temperature for 1 h. The reaction mixture was added dropwise to a mixture of EtOAc and K₂CO₃ (30 g) in water (200 mL). The two layers were separated, and the aqueous phase was extracted with EtOAc. The combined organic layers were washed with sat aq NaHCO₃ and brine, dried over Na₂SO₄ and evaporated. The crude product was purified by silica gel chromatography using EtOAc in heptanes (0 to 20%), affording 4-bromo-3-fluoroisoquinoline (439 mg, 86% yield) as white solid. ¹H NMR (400 MHz, CDCl3) δ ppm 7.64 (ddd, J=8.1, 7.0, 1.0 Hz, 1H), 7.77-7.93 (m, 1H), 8.02 (d, J=8.3 Hz, 1H), 8.20 (dd, J=8.7, 0.9 Hz, 1H), 8.90 (s, 1H). ¹⁹F NMR (377 MHz, CDCl3) δ ppm −72.12 (br. s, 1 F). MS: [M+1]: 226.0, 228.0.

### Preparation of Intermediate Q

Bromination of 5-bromo-2-methylpyridin-3-amine followed by formation of the fused pyrazole ring and N-methylation gives access to the required dibromo fused pyridine as illustrated in Scheme 6.

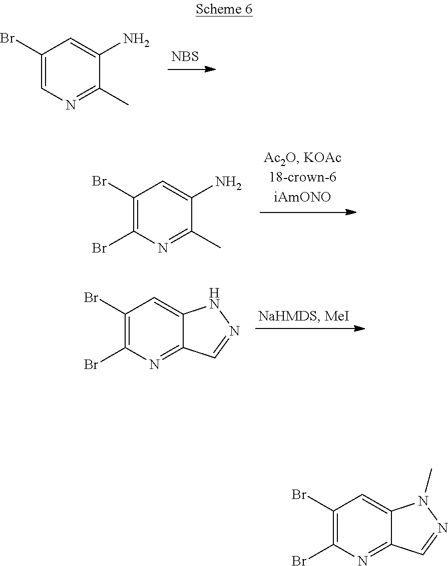

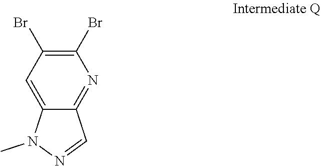

Step 1. To a solution of 5-bromo-2-methylpyridin-3-amine (5.00 g, 26.7 mmol) in acetonitrile (50 mL) was added portion wise NBS (5.00 g, 28.1 mmol) at 18-22° C. Once the addition was complete, the suspension was stirred at RT 1 h. The product was filtered (first crop) and the filtrate was evaporated to one third the initial volume. The solution was partitioned between DCM and water and the organic phase was washed with brine, dried over sodium sulfate, filtered, and adsorbed on silica. The crude product was purified by silica gel chromatography using EtOAc in heptanes (10 to 30%), providing a second crop of material which was merged with the first crop, affording 5,6-dibromo-2-methylpyridin-3-amine (5.46 g, 76% yield) as a solid.

Step 2. To a solution of 5,6-dibromo-2-methylpyridin-3-amine (3.00 g, 11.3 mmol) in CHCl₃ (70 mL) were added potassium acetate (1.33 g, 13.5 mmol) and acetic anhydride (4.27 mL, 45.1 mmol). The reaction mixture was heated to reflux for 3 h to get a suspension then cooled to RT and 18-Crown-6 (298 mg, 1.13 mmol) and isoamylnitrite (1.52 mL, 11.3 mmol) were added. The suspension was heated to reflux for 12 h. Methanol (20 mL) and a solution of K₂CO₃ (8.0 g) in 20 mL of water were added. The biphasic solution was stirred at RT for 3 h. The precipitate was collected by filtration and the solid was dissolved into warm EtOAc (˜200 mL) and filtered through a pad of silica gel. The filter cake was rinsed with EtOAc. The filtrate was evaporated to get 5,6-dibromo-1H-pyrazolo[4,3-b]pyridine (1.11 g, 35% yield) as a beige solid.

Step 3. To a solution of 5,6-dibromo-1H-pyrazolo[4,3-b]pyridine (813 mg, 2.94 mmol) in N,N-dimethylformamide (16 mL, 207 mmol) was added dropwise a solution of NaHMDS in THE (1M, 3.8 mL, 3.8 mmol) at 0-5° C. under nitrogen. After 5 minutes of stirring, iodomethane (240 μL, 3.86 mmol) was added and the reaction mixture was stirred at room temperature for 1 h. The reaction mixture was partitioned between EtOAc and water/brine (3:1, 140 mL). The aqueous layer was extracted with EtOAc once and the combined organic phases were washed with water and brine, dried over Na₂SO₄ and evaporated. The crude product was purified by silica gel chromatography using EtOAc in heptanes (5 to 100%). The major, less polar regioisomer was collected to get 5,6-dibromo-1-methyl-1H-pyrazolo[4,3-b]pyridine (432 mg, 50% yield). ¹H NMR (400 MHz, CDCl3) δ ppm 4.08 (s, 3H), 8.06 (s, 1H), 8.14 (s, 1H). MS: [M+1]: 292.0.

### Preparation of Intermediates R-Z

Condensation of a 2,3-diaminoaryl compound with diethyloxalate followed by chlorination of the resulting dione intermediate gives access to the required substituted fused 2,3-dichloropyrazine as illustrated in the Scheme 4.

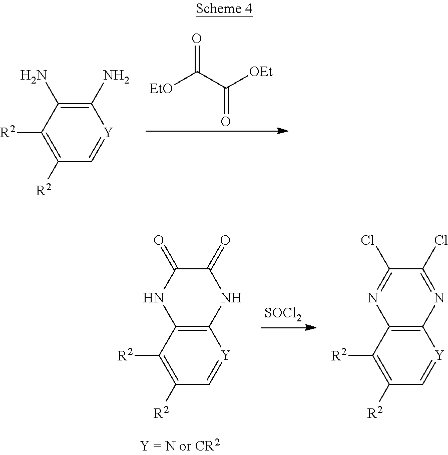

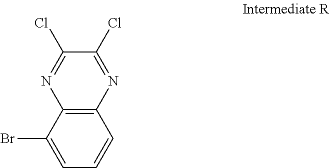 [SMILES: Clc1nc2cccc(Br)c2nc1Cl]

Step 1. A solution of 3-bromobenzene-1,2-diamine (37.50 g, 200.5 mmol) in diethyl oxalate (205 g, 1.40 mol, 190 mL) was refluxed for 4 h. The reaction mixture was allowed to cool to room temperature, after which EtOAc (500 mL) was added. The precipitate was filtered, washed with EtOAc three times and dried under vacuum to give 5-bromo-1,4-dihydroquinoxaline-2,3-dione (42 g, 87% yield) as a brown powder.

Step 2. To a mixture of 5-bromo-1,4-dihydroquinoxaline-2,3-dione (42 g, 174 mmol) in thionyl chloride (623 g, 5.24 mol, 380 mL) was added DMF (2.36 g, 32.3 mmol, 2.5 mL). The reaction mixture was heated under reflux for 4 h, cooled to room temperature, and poured very slowly into an ice/water bath with vigorous stirring. The precipitate was filtered, dissolved in EtOAc (750 mL), and dried with Na₂SO₄. 40 g of silica gel was added and the mixture was evaporated to afford a brown residue that was purified by chromatography on silica gel (0-20% EtOAc in hexanes) to provide 5-bromo-2,3-dichloro-quinoxaline (35.4 g, 73% yield) as a white solid. ¹H NMR (400 MHz, DMSO-d6) δ 8.24-8.19 (m, 1H), 8.05-8.00 (m, 1H), 7.81-7.74 (m, 1H).

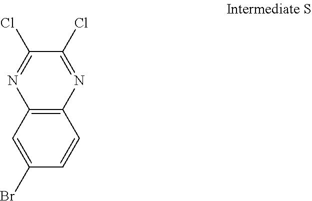 [SMILES: Clc1nc2ccc(Br)cc2nc1Cl]

6-bromo-2,3-dichloro-quinoxaline (15.5 g) as a light orange-brown solid was obtained in two steps from 4-bromobenzene-1,2-diamine. ¹H NMR (400 MHz, DMSO-d6) δ 8.29 (dd, J=2.1, 0.5 Hz, 1H), 8.00 (d, J=2.1 Hz, 1H), 7.97 (d, J=0.5 Hz, 1H).

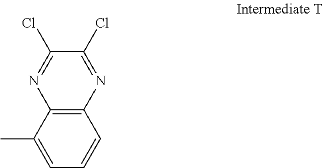

2,3-dichloro-5-methyl-quinoxaline (6.5 g) was obtained as a white solid in two steps from 3-methylbenzene-1,2-diamine. ¹H NMR (400 MHz, Chloroform-d) δ 7.81 (dd, J=8.4, 1.4, 1H), 7.65 (dd, J=8.3, 7.1 Hz, 1H), 7.59 (m, 1H), 2.72 (s, 3H). MS: [M+1]: 213.1.

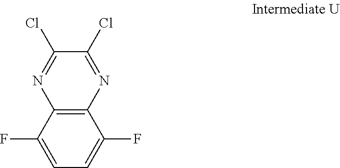 [SMILES: Fc1ccc(F)c2nc(Cl)c(Cl)nc12]

2,3-dichloro-5,8-difluoro-quinoxaline (1.1 g) was obtained as a white solid in two steps from 3,6-difluorobenzene-1,2-diamine. ¹H NMR (400 MHz, Chloroform-d) δ 7.55-7.35 (m, 2H).

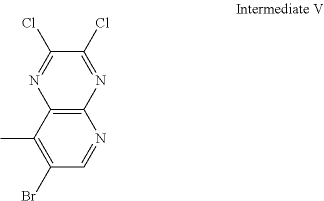

7-bromo-2,3-dichloro-8-methyl-pyrido[2,3-b]pyrazine (1.6 g) was obtained as a white solid in two steps from 5-bromo-4-methyl-pyridine-2,3-diamine. ¹H NMR (400 MHz, Chloroform-d) δ 9.19 (s, 1H), 2.82 (s, 3H). MS: [M+1]: 293.9.

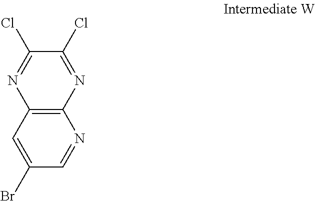 [SMILES: Clc1nc2cc(Br)cnc2nc1Cl]

7-bromo-2,3-dichloro-pyrido[2,3-b]pyrazine (14.2 g) was obtained as a white solid in two steps from 5-bromo-pyridine-2,3-diamine. ¹H NMR (400 MHz, DMSO-d6) δ 9.20 (d, J=2.4 Hz, 1H), 8.89 (d, J=2.4 Hz, 1H). MS: [M+1]: 280.0.

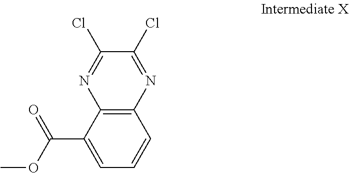 [SMILES: COC(=O)c1cccc2nc(Cl)c(Cl)nc12]

Methyl 2,3-dichloroquinoxaline-5-carboxylate (3.02 g) was obtained as a light reddish brown solid in two steps from methyl 2,3-diaminobenzoate. ¹H NMR (400 MHz, DMSO-d6) δ 8.27 (dd, J=8.4, 1.4 Hz, 1H), 8.22 (dd, J=7.3, 1.4 Hz, 1H), 8.00 (dd, J=8.4, 7.3 Hz, 1H), 3.95 (s, 3H). MS: [M+1]: 257.1.

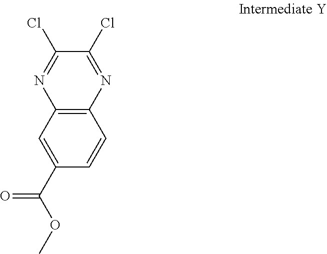 [SMILES: COC(=O)c1ccc2nc(Cl)c(Cl)nc2c1]

Methyl 2,3-dichloroquinoxaline-6-carboxylate (10.9 g) was obtained as a white solid in two steps from methyl 3,4-diaminobenzoate. ¹H NMR (400 MHz, DMSO-d6) δ 8.52 (dd, J=1.9, 0.6 Hz, 1H), 8.34 (dd, J=8.7, 1.9 Hz, 1H), 8.18 (dd, J=8.8, 0.6 Hz, 1H), 3.96 (s, 3H).

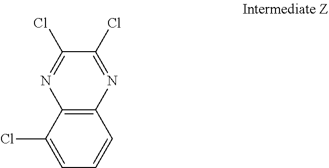 [SMILES: Clc1nc2cccc(Cl)c2nc1Cl]

2,3,5-trichloroquinoxaline (5.5 g) was obtained as a white solid in two steps from 3-chlorobenzene-1,2-diamine. ¹H NMR (400 MHz, Chloroform-d) δ 7.92 (dd, J=8.4, 1.3 Hz, 1H), 7.85 (dd, J=7.7, 1.3 Hz, 1H), 7.70 (m, 1H).

### Chiral Separation of Selected Compounds

Racemic mixtures of atropisomers were separated using chiral SFC methods on a Mettler Toledo Minigram SFC (MTM), a Waters Prep 15 SFC-MS (WP15), a Waters Prep 100 SFC-MS (WP100) or a Pic Solution Hybrid 10-150 (PSH) (Table 4). An appropriate column was selected to achieve a satisfactory resolution of the peaks. The appropriate fractions for each peak were combined, concentrated and usually taken in a mixture of water and a suitable water miscible organic solvent such as EtOH, IPA, CH₃CN or a mixture thereof and freeze-dried. The separated products were reanalyzed by chiral SFC to assess chiral purity.

C1A is Phenomenex Lux Cellulose-2, 10×250 mm, 5 μm; C1B is Phenomenex Lux Cellulose-2, 30×250 mm, 5 μm; C2 is Chiral Technologies IA, 10×250 mm, 5 μm; C3 is Chiral Technologies IC, 10×250 mm, 5 μm; C4 is Chiral Technologies ID, 10×250 mm, 5 μm; C5 is Chiral Technologies IG, 10×250 mm, 5 μm; C6 is Chiral Technologies AS, 10×250 mm, 5 μm; C7 is Phenomenex Lux Cellulose-4, 10×250 mm, 5 μm; C8 is Phenomenex Lux Cellulose-1, 21.2×250 mm, 5 μm.

Structural assignments of the separated atropisomers were confirmed by biological activity where the biologically active enantiomer was assigned to have the (S) configuration, which was confirmed by X-ray crystallography of key compounds.

TABLE 4

| | Peak 1 | Peak 2 | | | |
|---|---|---|---|---|---|
| Mixture | Cmpd # | Cmpd # | | | Eluent (%, Flow Rate |
| Cmpd # | (RT) | (RT) | Instrument | Column | (mL/min)) |
| 1 | 2 | 3 | MTM | C3 | MeOH (30%, 10) |
| | (7.67 min) | (11.20 min) | | | |
| 5 | 6 | 7 | MTM | C1A | MeOH + 10 mM Ammonium |
| | (7.03 min) | (13.10 min) | | | Formate (55%, 10) |
| 24 | 25 | 26 | MTM | C2 | IPA + 10 mM Ammonium |
| | (3.06 min) | (5.38 min) | | | Formate (40%, 10) |
| 29 | 30 | 31 | MTM | C3 | MeOH + 10 mM Ammonium |
| | (6.00 min) | (8.31 min) | | | Formate (35%, 10) |
| 40 | 41 | 42 | MTM | C6 | MeOH + 10 mM Ammonium |
| | (5.88 min) | (8.13 min) | | | Formate (35%, 10) |
| 55 | 56 | 57 | MTM | C1A | 1:1 ACN/EtOH (55%, 10) |
| | (6.81 min) | (10.03 min) | | | |

### Example 2. Enzymatic Assay

Detection of Myt1 kinase activity utilized a recombinant human Myt1 kinase assay measuring the hydrolysis of ATP using a commercially available ADP-Glo Assay (ADP-Glo™ Kinase Assay from Promega, 10 000 assays, #V9102). Briefly, 5 μL recombinant human Myt1 (full length PKMYT1 recombinant human protein expressed in insect cells from Thermo Fisher #A33387; ˜80% purity) was prepared in reaction buffer (70 mM HEPES, 3 mM MgCl₂, 3 mM MnCl₂, 50 μg/ml PEG 20000, 3 μM Na-orthovanadate, 1.2 mM DTT) and added to 384 well white polystyrene, flat bottom well, non-treated, microplate (Corning #3572). After this, 5 μL of compounds (diluted in reaction buffer to 0.5% DMSO) was added to the microplate and the plate was spun briefly and incubated at 22° C. for 15 minutes. Ultra-Pure Adenosine Triphosphate (ATP) solution (ADP-Glo kit from Promega) was diluted in reaction buffer and 5 μL was added to the microplate, spun down briefly and incubated for 60 minutes at 30° C. The final Myt1 enzyme concentration was 18 nM and the final ATP concentration was 10 μM. After the 60-minute incubation, 15 μL of ADP-Glo reagent was added and the plate was spun briefly and sealed and incubated in the dark for 40 minutes at 22° C. Following this, 30 μL of kinase detection reagent was added per well and the plate was spun briefly, sealed and incubated for 45-60 minutes at 22° C. in the dark. Luminescence was read using the Envision (250 ms integration). The IC₅₀ and the % max inhibition were calculated for each inhibitor compound tested.

Exemplary prepared compounds and their activities were shown in Table 5 below.

TABLE 5

| | | Myt1 | MS | |
|---|---|---|---|---|
| | | IC₅₀ | (+ESI) | |
| Cpd. | Method | (nM) | [M + 1] | NMR |
| 1 | A | <10 | m/z | ¹H NMR (400 MHz, DMSO-d6) δ 9.65 (brs, 1H), |
| | | | 348.2 | 8.01 (brs, 2H), 7.95 (ddd, J = 8.3, 1.5, 0.5 Hz, 1H), |
| | | | | 7.81-7.71 (m, 2H), 7.57 (ddd, J = 8.4, 6.9, 1.5 Hz, |
| | | | | 1H), 7.46 (ddd, J = 8.3, 6.9, 1.5 Hz, 1H), 7.38 (br s, |
| | | | | 1H), 7.12 (dt, J = 8.3, 0.7 Hz, 1H), 6.99 (d, J = 8.3 |
| | | | | Hz, 1H), 1.83 (s, 3H), 1.76 (s, 3H). |
| 2 | A | 5000 | m/z | ¹H NMR (400 MHz, DMSO-d6) δ 9.65 (s, 1H), 7.99 |
| | | | 348.3 | (brs, 3H), 7.97-7.90 (m, 1H), 7.81-7.69 (m, 2H), |
| | | | | 7.58 (ddd, J = 8.4, 6.9, 1.5 Hz, 1H), 7.46 (ddd, J = |
| | | | | 8.3, 6.9, 1.5 Hz, 1H), 7.37 (brs, 1H), 7.12 (dt, J = |
| | | | | 8.3, 0.8 Hz, 1H), 6.98 (d, J = 8.3 Hz, 1H), 1.83 |
| | | | | (s, 3H), 1.75 (s, 3H). |
| 3 | A | <10 | m/z | ¹H NMR (400 MHz, DMSO-d6) δ 9.65 (s, 1H), 7.99 |
| | | | 348.3 | (brs, 3H), 7.97-7.90 (m, 1H), 7.81-7.69 (m, 2H), |
| | | | | 7.58 (ddd, J = 8.4, 6.9, 1.5 Hz, 1H), 7.46 (ddd, J = |
| | | | | 8.3, 6.9, 1.5 Hz, 1H), 7.37 (brs, 1H), 7.12 (dt, J = |
| | | | | 8.3, 0.8 Hz, 1H), 6.98 (d, J = 8.3 Hz, 1H), 1.83 (s, |
| | | | | 3H), 1.75 (s, 3H). |
| 4 | A | 130 | m/z | ¹H NMR (500 MHz, DMSO-d6) δ 8.16 (brs, 2H), |
| | | | 354.1 | 7.93 (dd, J = 8.4, 1.4 Hz, 1H), 7.78 (dd, J = 8.3, 1.4 |
| | | | | Hz, 1H), 7.73 (br s, 1H), 7.57 (ddd, J = 8.4, 6.9, 1.5 |
| | | | | Hz, 1H), 7.54 (d, J = 8.7 Hz, 1H), 7.46 (ddd, J = 8.3, |
| | | | | 6.9, 1.5 Hz, 1H), 7.36 (brs, 1H), 7.10-7.00 (m, 2H). |
| 5 | A from arylamine | <10 | m/z | ¹H NMR (400 MHz, DMSO-d6) δ 10.56 (brs, 1H), |
| | AA5 | | 368.3 | 8.17 (brs, 2H), 7.95 (ddd, J = 8.3, 1.5, 0.5 Hz, 1H), |
| | | | | 7.78 (ddd, J = 8.2, 1.5, 0.5 Hz, 1H), 7.75 (brd, J = |
| | | | | 3.1 Hz, 1H), 7.58 (ddd, J = 8.4, 6.9, 1.5 Hz, 1H), |
| | | | | 7.47 (ddd, J = 8.4, 6.9, 1.5 Hz, 1H), 7.39 (brs, 1H), |
| | | | | 7.28 (dd, J = 8.5, 0.8 Hz, 1H), 7.16 (d, J = 8.5 Hz, |
| | | | | 1H), 1.94 (s, 3H). |
| 6 | A from arylamine | 8250 | m/z | ¹H NMR (400 MHz, DMSO-d6) δ 10.56 (brs, 1H), |
| | AA5 | | 368.3 | 8.17 (brs, 2H), 7.95 (ddd, J = 8.3, 1.5, 0.5 Hz, 1H), |
| | | | | 7.78 (ddd, J = 8.2, 1.5, 0.5 Hz, 1H), 7.75 (brd, J = |
| | | | | 3.1 Hz, 1H), 7.58 (ddd, J = 8.4, 6.9, 1.5 Hz, 1H), |
| | | | | 7.47 (ddd, J = 8.4, 6.9, 1.5 Hz, 1H), 7.39 (brs, 1H), |
| | | | | 7.28 (dd, J = 8.5, 0.8 Hz, 1H), 7.16 (d, J = 8.5 Hz, |
| | | | | 1H), 1.94 (s, 3H). |
| 7 | A from arylamine | <10 | m/z | ¹H NMR (400 MHz, DMSO-d6) δ 10.56 (brs, 1H), |
| | AA5 | | 368.3 | 8.17 (brs, 2H), 7.95 (ddd, J = 8.3, 1.5, 0.5 Hz, 1H), |
| | | | | 7.78 (ddd, J = 8.2, 1.5, 0.5 Hz, 1H), 7.75 (brd, J = |
| | | | | 3.1 Hz, 1H), 7.58 (ddd, J = 8.4, 6.9, 1.5 Hz, 1H), |
| | | | | 7.47 (ddd, J = 8.4, 6.9, 1.5 Hz, 1H), 7.39 (brs, 1H), |
| | | | | 7.28 (dd, J = 8.5, 0.8 Hz, 1H), 7.16 (d, J = 8.5 Hz, |
| | | | | 1H), 1.94 (s, 3H). |
| 8 | A from arylamine | 289 | m/z | ¹H NMR (400 MHz, DMSO-d6) δ 13.73 (brs, 1H), |
| | AA10 | | 378.2 | 8.05 (brs, 2H), 7.95 (ddd, J = 8.3, 1.5, 0.5 Hz, 1H), |
| | | | | 7.86 (dd, J = 8.6, 0.7 Hz, 1H), 7.79 (brd, J = 3.0 Hz, |
| | | | | 1H), 7.71-7.63 (m, 2H), 7.57 (ddd, J = 8.4, 6.9, 1.5 |
| | | | | Hz, 1H), 7.47-7.40 (m, 2H), 7.38 (br s, 1H). |
| 9 | A from arylamine | 281 | m/z | ¹H NMR (400 MHz, DMSO-d6) δ 13.03 (brs, 1H), |
| | AA9 | | 362.2 | 8.19 (br s, 2H), 7.96 (d, J = 8.3 Hz, 1H), 7.84-7.63 |
| | | | | (m, 4H), 7.62 - 7.52 (m, 1H), 7.50-7.33 (m, 3H). |
| 10 | A | 182 | m/z | ¹H NMR (400 MHz, DMSO-d6) δ 13.45 (s, 1H), 8.07 |
| | | | 344.1 | (brs, 2H), 7.96 (ddd, J = 8.3, 1.5, 0.5 Hz, 1H), 7.89- |
| | | | | 7.75 (m, 3H), 7.69 (ddd, J = 8.2, 1.5, 0.5 Hz, 1H), |
| | | | | 7.63-7.51 (m, 2H), 7.44 (ddd, J = 8.3, 6.9, 1.5 Hz, |
| | | | | 1H), 7.42 - 7.38 (m, 1H), 7.35 (dd, J = 7.3, 0.7 Hz, |
| | | | | 1H). |
| 11 | A from arylamine | 2170 | m/z | |
| | AA13 | | 374.4 | |
| 12 | A from arylamine | 98 | m/z | |
| | AA11 | | 362.3 | |
| 13 | A from arylamine | 427 | m/z | |
| | AA12 | | 362.3 | |
| 14 | B from arylamine | 10 | m/z | ¹H NMR (400 MHz, DMSO-d6) δ 10.23 (brs, 1H), |
| | AA6 | | 368.3 | 8.18 (br s, 2H), 7.95 (dd, J = 8.5, 1.4 Hz, 1H), 7.82- |
| | | | | 7.76 (m, 1H), 7.75 (brd, J = 2.7 Hz, 1H), 7.58 (ddd, |
| | | | | J = 8.4, 6.9, 1.5 Hz, 1H), 7.47 (ddd, J = 8.3, 6.9, 1.5 |
| | | | | Hz, 1H), 7.42 - 7.34 (m, 2H), 7.09 (d, J = 8.8 Hz, |
| | | | | 1H), 1.86 (s, 3H). |
| 15 | B from arylamine | <10 | m/z | ¹H NMR (400 MHz, DMSO-d6) 6 8.33 (brs, 2H), |
| | AA7 | | 388.1 | 7.95 (dd, J = 8.3, 1.4 Hz, 1H), 7.79 (dd, J = 8.3, 1.4 |
| | | | | Hz, 1H), 7.74 (brs, J = 3.1 Hz, 1H), 7.60 (ddd, J = |
| | | | | 8.4, 6.9, 1.5 Hz, 1H), 7.56 (d, J = 9.0 Hz, 1H), 7.48 |
| | | | | (ddd, J = 8.4, 6.9, 1.5 Hz, 1H), 7.41 (brd, J = 3.1 |
| | | | | Hz, 1H), 7.26 (d, J = 9.0 Hz, 1H). |
| 16 | C | 94 | m/z | ¹H NMR (400 MHz, DMSO-d6) 6 9.64 (s, 1H), 7.72 |
| | | | 356.1 | (s, 2H), 7.24 (dd, J = 8.3, 0.8 Hz, 1H), 7.11 (s, 4H), |
| | | | | 6.87 (dd, J = 8.3, 2.6 Hz, 1H), 6.68 (d, J = 2.5 Hz, |
| | | | | 1H), 1.80 (s, 3H). |
| 17 | D | 29 | m/z | ¹H NMR (500 MHz, DMSO-d6) δ 9.80 (s, 1H), 8.29 |
| | | | 333.3 | (d, J = 3.7 Hz, 1H), 7.90 (d, J = 8.5 Hz, 1H), 7.85 |
| | | | | (dd, J = 8.1, 1.4 Hz, 1H), 7.52 (br s + ddd, J = 8.4, |
| | | | | 6.8, 1.5 Hz, 3H), 7.36-7.31 (m, 2H), 7.29 (ddd, J = |
| | | | | 8.1,6.8, 1.3 Hz, 1H), 7.18 (d, J = 3.5 Hz, 1H), 6.96 |
| | | | | (dd, J = 8.4, 2.6 Hz, 1H), 6.79 (d, J = 2.5 Hz, 1H), |
| | | | | 1.87 (s, 3H). |
| 18 | E | 12 | m/z | ¹H NMR (500 MHz, DMSO-de) δ 9.65 (s, 1H), 8.44 |
| | | | 333.2 | (s, 1H), 7.85 (dd, J= 8.1, 1.5 Hz, 1H), 7.70 (d, J = |
| | | | | 8.3 Hz, 1H), 7.45 (br s, 2H), 7.45 - 7.41 (m, 1H), |
| | | | | 7.38 (ddd, J = 8.1,6.7, 1.4 Hz, 1H), 7.29 (d, J= 8.4 |
| | | | | Hz, 1H), 6.92 (dd, J= 8.4, 2.5 Hz, 1H), 6.86 (brs, |
| | | | | 2H), 6.73 (d, J = 2.6 Hz, 1H), 1.84 (s, 3H). |
| 19 | F | 4060 | m/z | ¹H NMR (400 MHz, DMSO-d6) δ 9.72 (s, 1H), 8.20 |
| | | | 362.32 | (d, J = 5.6 Hz, 1H), 7.95 (d, J = 7.6 Hz, 1H), 7.78 (d, |
| | | | | J = 7.6 Hz, 1H), 7.58 (t, J = 7.2 Hz, 1H), 7.46 (t, J = |
| | | | | 7.2 Hz, 1H), 7.30 (d, J = 8.4 Hz, 1H), 6.94 (dd, J = |
| | | | | 8.4, 2.4 Hz, 1H), 6.81 (d, J = 2.4 Hz, 1H), 3.48 - |
| | | | | 3.40 (m, 2H), 1.88 (s, 3H), 1.23 (t, J = 7.2 Hz, 3H). |
| 20 | F | 7580 | m/z | ¹H NMR (400 MHz, DMSO-de) δ 9.75 (bs, 1H), 8.35 |
| | | | 374.32 | (s, 1H), 8.18 (bs, 1H), 8.00 (d, J= 7.6 Hz, 1H), 7.80 |
| | | | | (d, J = 7.6 Hz, 1H), 7.60 (t, J = 7.2 Hz, 1H), 7.49 (t, J |
| | | | | = 7.2 Hz, 1H), 7.31 (d, J = 8.4 Hz, 1H), 6.96 (d, J = |
| | | | | 8.0 Hz, 1H), 6.83 (s, 1H), 2.88 (s, 1H), 1.89 (s, 3H), |
| | | | | 0.82 (d, J = 6.0 Hz, 2H), 0.67 (bs, 2H). |
| 21 | F | 1160 | m/z | ¹H NMR (400 MHz, DMSO-d6) δ 9.74 (s, 1H), 8.40 |
| | | | 378.26 | (d, J = 5.2 Hz, 1H), 8.04-7.92 (m, 3H), 7.79 (d, J = |
| | | | | 8.0 Hz, 1H), 7.59 (t, J = 7.6 Hz, 1H), 7.48 (t, J = 7.6 |
| | | | | Hz, 1H), 7.32 (d, J = 8.0 Hz, 1H), 6.96 (d, J = 8.0 |
| | | | | Hz, 1H), 6.83 (s, 1H), 4.91 (t, J = 4.4 Hz, 1H), 3.62 |
| | | | | (t, J = 6.0 Hz, 2H), 3.52 (t, J = 5.2 Hz, 2H), 1.89 (s, |
| | | | | 3H). |
| 22 | F | 690 | m/z | ¹HNMR(400 MHz, DMSO-d6) δ 9.75 (s, 1H), 8.11- |
| | | | 348.46 | 7.96 (m, 3H), 7.79 (d, J = 8.4 Hz, 1H), 7.59 (t, J = |
| | | | | 7.6 Hz, 1H), 7.50-7.56 (m, 1H), 7.32 (d, J = 8.0 Hz, |
| | | | | 1H), 6.96 (dd, J = 8.4, 2.4 Hz, 1H), 6.82 (d, J = 2.4 |
| | | | | Hz, 1H), 2.97 (d, J = 4.4 Hz, 3H), 1.89 (s, 3H). |
| 23 | A then G | 323 | m/z | ¹H NMR (400 MHz, DMSO-d6) δ 8.91 (brs, 1H), |
| | | | 319.1 | 8.03 (s, 1H), 7.53 - 7.40 (m, 1H), 7.32-7.20 (m, |
| | | | | 2H), 7.09 - 6.90 (m, 3H), 6.46 (d, J = 8.2 Hz, 1H), |
| | | | | 6.17-6.00 (m, 2H), 1.14 (s, 3H). |
| 24 | A then G | 11 | m/z | ¹H NMR (400 MHz, DMSO-d6) δ 9.67 (s, 1H), 8.82 |
| | | | 333.4 | (s, 1H), 8.37-8.24 (m, 1H), 8.11-8.04 (m, 2H), |
| | | | | 7.91-7.73 (m, 3H), 7.11 (dt, J = 8.3, 0.7 Hz, 1H), |
| | | | | 6.97 (d, J = 8.3 Hz, 1H), 1.81 (s, 3H), 1.71 (s, 3H). |
| 25 | A then G | 5000 | m/z | ¹H NMR (400 MHz, DMSO-d6) δ 9.67 (s, 1H), 8.82 |
| | | | 333.4 | (s, 1H), 8.37-8.24 (m, 1H), 8.11-8.04 (m, 2H), |
| | | | | 7.91 - 7.73 (m, 3H), 7.11 (dt, J = 8.3, 0.7 Hz, 1H), |
| | | | | 6.97 (d, J = 8.3 Hz, 1H), 1.81 (s, 3H), 1.71 (s, 3H). |
| 26 | A then G | <10 | m/z | ¹H NMR (400 MHz, DMSO-d6) δ 9.67 (s, 1H), 8.82 |
| | | | 333.4 | (s, 1H), 8.37-8.24 (m, 1H), 8.11-8.04 (m, 2H), |
| | | | | 7.91-7.73 (m, 3H), 7.11 (dt, J = 8.3, 0.7 Hz, 1H), |
| | | | | 6.97 (d, J = 8.3 Hz, 1H), 1.81 (s, 3H), 1.71 (s, 3H). |
| 28 | A from | <10 | m/z | ¹H NMR (400 MHz, DMSO-d6) δ 9.66 (s, 1H), 8.22 |
| | intermediate U | | 384.2 | (s, 2H), 7.63 (d, J = 3.1 Hz, 1H), 7.46 (d, J = 3.1 Hz, |
| | | | | 1H), 7.36 (ddd, J = 10.3, 8.7, 4.3 Hz, 1H), 7.23 (ddd, |
| | | | | J = 10.2, 8.7, 4.2 Hz, 1H), 7.10 (d, J = 8.3 Hz, 1H), |
| | | | | 6.96 (d, J = 8.3 Hz, 1H), 1.80 (s, 3H), 1.72 (s, 3H). |
| 29 | A from | <10 | m/z | ¹H NMR (400 MHz, DMSO-d6) δ 9.61 (s, 1H), 8.12 |
| | intermediate R | | 428.0 | (m, 2H), 7.89 (dd, J = 7.6, 1.3 Hz, 1H), 7.82 (d, J = |
| | | | | 3.3 Hz, 1H), 7.77 (dd, J = 8.3, 1.3 Hz, 1H), 7.48 (d, J |
| | | | | = 3.3 Hz, 1H), 7.33 (dd, J = 8.3, 7.6 Hz, 1H), 7.08 |
| | | | | (dt, J = 8.3, 0.8 Hz, 1H), 6.94 (d, J = 8.3 Hz, 1H), |
| | | | | 1.79 (s, 3H), 1.72 (s, 3H). |
| 30 | A from | 1180 | m/z | ¹H NMR (400 MHz, DMSO-d6) δ 9.61 (s, 1H), 8.12 |
| | intermediate R | | 428.0 | (m, 2H), 7.89 (dd, J = 7.6, 1.3 Hz, 1H), 7.82 (d, J = |
| | | | | 3.3 Hz, 1H), 7.77 (dd, J = 8.3, 1.3 Hz, 1H), 7.48 (d, |
| | | | | J = 3.3 Hz, 1H), 7.33 (dd, J = 8.3, 7.6 Hz, 1H), 7.08 |
| | | | | (dt, J = 8.3, 0.8 Hz, 1H), 6.94 (d, J = 8.3 Hz, 1H), |
| | | | | 1.79 (s, 3H), 1.72 (s, 3H). |
| 31 | A from | <10 | m/z | ¹H NMR (400 MHz, DMSO-d6) δ 9.61 (s, 1H), 8.12 |
| | intermediate R | | 428.0 | (m, 2H), 7.89 (dd, J = 7.6, 1.3 Hz, 1H), 7.82 (d, J = |
| | | | | 3.3 Hz, 1H), 7.77 (dd, J = 8.3, 1.3 Hz, 1H), 7.48 (d, |
| | | | | J = 3.3 Hz, 1H), 7.33 (dd, J = 8.3, 7.6 Hz, 1H), 7.08 |
| | | | | (dt, J = 8.3, 0.8 Hz, 1H), 6.94 (d, J = 8.3 Hz, 1H), |
| | | | | 1.79 (s, 3H), 1.72 (s, 3H). |
| 32 | H on compound 29 | <10 | m/z | ¹H NMR (400 MHz, DMSO-d6) δ 9.62 (s, 1H), 8.31 |
| | | | 373.2 | (s, 2H), 8.18-8.02 (m, 2H), 7.69 (d, J = 3.1 Hz, |
| | | | | 1H), 7.55 (d, J = 6.7 Hz, 1H), 7.51 (dd, J = 8.4, 7.3 |
| | | | | Hz, 1H), 7.08 (d, J = 8.3 Hz, 1H), 6.95 (d, J = 8.3 |
| | | | | Hz, 1H), 1.79 (s, 3H), 1.72 (s, 3H). |
| 33 | I on compound 29 | 16 | m/z | ¹H NMR (400 MHz, DMSO-d6) δ 9.65 (s, 1H), 8.02 |
| | | | 412.3 | (s, 2H), 7.88 (d, J = 3.4 Hz, 1H), 7.68 (dd, J = 8.3, |
| | | | | 1.4 Hz, 1H), 7.59 (dd, J = 7.3, 1.4 Hz, 1H), 7.54- |
| | | | | 7.47 (m, 1H), 7.33 (dd, J = 8.3, 7.3 Hz, 1H), 7.08 (dt, |
| | | | | J = 8.3, 0.7 Hz, 1H), 6.94 (d, J = 8.3 Hz, 1H), 1.82- |
| | | | | 1.75 (m, 3H), 1.71 (s, 3H), 1.61 (tt, J = 8.2, 5.0 Hz, |
| | | | | 1H), 0.98-0.88 (m, 2H), 0.81 - 0.69 (m, 2H). |
| 34 | J on compound 29 | 12 | m/z | ¹H NMR (400 MHz, DMSO-d6) δ 9.65 (s, 1H), 7.95 |
| | | | 430.3 | (s, 2H), 7.74-7.60 (m, 2H), 7.44-7.33 (m, 3H), |
| | | | | 7.08 (dt, J = 8.3, 0.8 Hz, 1H), 6.94 (d, J = 8.3 Hz, |
| | | | | 1H), 5.91 (m, 1H), 4.26 (m, 2H), 3.85 (t, J = 5.3 Hz, |
| | | | | 2H), 2.66 (m, 2H), 1.79 (d, J = 0.7 Hz, 3H), 1.71 (s, |
| | | | | 3H). |
| 35 | J on compound 29 | <10 | m/z | ¹H NMR (400 MHz, DMSO-d6) δ 9.63 (s, 1H), 7.95 |
| | | | 428.3 | (s, 2H), 7.76 (dd, J = 8.2, 1.6 Hz, 1H), 7.73-7.67 |
| | | | | (m, 2H), 7.64-7.31 (m, 6H), 7.19 (d, J = 3.4 Hz, |
| | | | | 1H), 7.09 (d, J = 8.3 Hz, 1H), 6.95 (d, J = 8.2 Hz, |
| | | | | 1H), 1.81 (s, 3H), 1.73 (s, 3H). |
| 36 | J on compound 29 | 17 | m/z | ¹H NMR (400 MHz, DMSO-d6) δ 9.63 (s, 1H), 7.95 |
| | | | 424.3 | (s, 2H), 7.76 (dd, J = 8.2, 1.6 Hz, 1H), 7.73 - 7.67 |
| | | | | (m, 2H), 7.64 - 7.31 (m, 6H), 7.19 (d, J = 3.4 Hz, |
| | | | | 1H), 7.09 (d, J = 8.3 Hz, 1H), 6.95 (d, J = 8.2 Hz, |
| | | | | 1H), 1.81 (s, 3H), 1.73 (s, 3H). |
| 37 | J on compound 29 | <10 | m/z | ¹H NMR (400 MHz, DMSO-d6) δ 9.61 (s, 1H), 7.95 |
| | | | 414.3 | (s, 2H), 7.73 - 7.57 (m, 2H), 7.47 (dd, J = 7.3, 1.6 |
| | | | | Hz, 1H), 7.43 - 7.32 (m, 2H), 7.08 (d, J = 8.3 Hz, |
| | | | | 1H), 6.94 (d, J = 8.3 Hz, 1H), 6.48 (m, 1H), 2.91 (m, |
| | | | | 2H), 2.54 (m, 2H), 1.98 (m, 2H), 1.79 (s, 3H), 1.72 |
| | | | | (s, 3H). |
| 38 | A from | <10 | m/z | ¹H NMR (400 MHz, DMSO-d6) δ 9.62 (s, 1H), 8.09 |
| | intermediate S | | 426.1 | (d, J = 2.3 Hz, 3H), 7.66 (d, J = 8.8 Hz, 1H), 7.63 |
| | | | | (dq, J = 4.9, 2.3 Hz, 1H), 7.50 (dd, J = 8.8, 2.3 Hz, |
| | | | | 1H), 7.43 - 7.32 (m, 1H), 7.06 (dt, J = 8.2, 0.8 Hz, |
| | | | | 1H), 6.92 (d, J = 8.3 Hz, 1H), 1.81 - 1.73 (m, 3H), |
| | | | | 1.69 (s, 3H). |
| 39 | H on compound 38 | <10 | m/z | ¹H NMR (400 MHz, DMSO-d6) δ 9.63 (s, 1H), 8.42 |
| | | | 373.2 | (d, J = 1.8 Hz, 1H), 8.39-8.12 (m, 2H), 7.87 (d, J = |
| | | | | 8.5 Hz, 1H), 7.71 (dd, J = 8.5, 1.9 Hz, 1H), 7.56 (s, |
| | | | | 2H), 7.08 (d, J = 8.3 Hz, 1H), 6.95 (d, J = 8.3 Hz, |
| | | | | 1H), 1.79 (s, 3H), 1.71 (s, 3H). |
| 40 | 1 on compound 38 | <10 | m/z | ¹H NMR (400 MHz, DMSO-d6) δ 9.63 (s, 1H), 8.03 |
| | | | 412.3 | (s, 2H), 7.86 (dd, J = 1.9, 0.5 Hz, 1H), 7.68 - 7.62 |
| | | | | (m, 2H), 7.39-7.29 (m, 2H), 7.08 (d, J = 8.4 Hz, |
| | | | | 1H), 6.94 (d, J = 8.4 Hz, 1H), 1.82-1.74 (m, 3H), |
| | | | | 1.70 (s, 3H), 1.55 (m, 1H), 0.92 - 0.82 (m, 2H), |
| | | | | 0.78-0.67 (m, 2H). |
| 41 | I on compound 38 | <10 | m/z | ¹H NMR (400 MHz, DMSO-d6) δ 9.63 (s, 1H), 8.03 |
| | | | 412.3 | (s, 2H), 7.86 (dd, J = 1.9, 0.5 Hz, 1H), 7.68-7.62 |
| | | | | (m, 2H), 7.39-7.29 (m, 2H), 7.08 (d, J = 8.4 Hz, |
| | | | | 1H), 6.94 (d, J = 8.4 Hz, 1H), 1.82-1.74 (m, 3H), |
| | | | | 1.70 (s, 3H), 1.55 (m, 1H), 0.92-0.82 (m, 2H), |
| | | | | 0.78-0.67 (m, 2H). |
| 42 | I on compound 38 | 1330 | m/z | ¹H NMR (400 MHz, DMSO-d6) δ 9.63 (s, 1H), 8.03 |
| | | | 412.3 | (s, 2H), 7.86 (dd, J = 1.9, 0.5 Hz, 1H), 7.68-7.62 |
| | | | | (m, 2H), 7.39-7.29 (m, 2H), 7.08 (d, J = 8.4 Hz, |
| | | | | 1H), 6.94 (d, J = 8.4 Hz, 1H), 1.82-1.74 (m, 3H), |
| | | | | 1.70 (s, 3H), 1.55 (m, 1H), 0.92-0.82 (m, 2H), |
| | | | | 0.78-0.67 (m, 2H). |
| 43 | I on compound 38 | <10 | m/z | ¹H NMR (400 MHz, DMSO-d6) δ 9.76 - 9.54 (m, |
| | | | 450.2 | 1H), 8.91 (d, J = 1.5 Hz, 1H), 8.68 (dd, J = 2.6, 1.5 |
| | | | | Hz, 1H), 8.67-8.58 (m, 1H), 8.34-7.99 (m, 3H), |
| | | | | 7.80 (d, J = 8.5 Hz, 1H), 7.70 (d, J = 13.4 Hz, 1H), |
| | | | | 7.63-7.48 (m, 1H), 7.41 (s, 1H), 7.13-7.05 (m, |
| | | | | 1H), 6.95 (d, J = 8.3 Hz, 1H), 1.85-1.76 (m, 3H), |
| | | | | 1.73 (s, 3H). |
| 44 | I on compound 38 | <10 | m/z | ¹H NMR (400 MHz, DMSO-d6) δ 9.65 (s, 1H), 8.06 |
| | | | 471.3 | (s, 2H), 7.96 (d, J = 1.9 Hz, 1H), 7.73-7.61 (m, |
| | | | | 2H), 7.39 (td, J = 8.9, 2.4 Hz, 2H), 7.08 (dt, J = 8.3, |
| | | | | 0.8 Hz, 1H), 6.94 (d, J = 8.3 Hz, 1H), 3.59 (t, J = 4.7 |
| | | | | Hz, 4H), 3.52 (s, 2H), 2.52 (dd, J = 5.6, 3.6 Hz, 4H), |
| | | | | 1.84-1.75 (m, 3H), 1.71 (s, 3H). |
| 45 | I on compound 38 | <10 | m/z | ¹H NMR (400 MHz, DMSO-d6) δ 9.66 (s, 1H), 8.06 |
| | | | 402.4 | (s, 2H), 7.93 (d, J = 1.8 Hz, 1H), 7.70 (d, J = 8.5 Hz, |
| | | | | 1H), 7.66 (d, J = 3.2 Hz, 1H), 7.44-7.33 (m, 2H), |
| | | | | 7.08 (d, J = 8.3 Hz, 1H), 6.94 (d, J = 8.3 Hz, 1H), |
| | | | | 4.31 (s, 2H), 1.79 (s, 3H), 1.71 (s, 3H). |
| 46 | Reduction of | <10 | m/z | ¹H NMR (400 MHz, DMSO-d6) δ 8.22-7.69 (m, |
| | compound 45 | | 406.2 | 5H), 7.64 (dd, J = 8.4, 1.5 Hz, 1H), 7.40-7.23 (m, |
| | | | | 2H), 7.07 (d, J = 8.3 Hz, 1H), 6.94 (dd, J = 8.3, 1.4 |
| | | | | Hz, 1H), 3.41 (t, J = 6.4 Hz, 4H), 2.76 (t, J = 7.6 Hz, |
| | | | | 3H), 1.78 (d, J = 6.1 Hz, 5H), 1.71 (d, J = 1.5 Hz, 3H). |
| 47 | J on compound 38 | <10 | m/z | ¹H NMR (400 MHz, DMSO-d6) δ 9.62 (s, 1H), 8.19 |
| | | | 424.3 | (d, J = 2.0 Hz, 1H), 8.13-7.91 (m, 2H), 7.87-7.67 |
| | | | | (m, 5H), 7.47 (m, 2H), 7.43-7.31 (m, 2H), 7.09 (d, |
| | | | | J = 8.2 Hz, 1H), 6.95 (d, J = 8.2 Hz, 1H), 1.81 (s, |
| | | | | 3H), 1.73 (s, 3H). |
| 48 | J on compound 38 | 11 | m/z | ¹H NMR (400 MHz, DMSO-d6) δ 9.64 (s, 1H), 8.31 |
| | | | 449.3 | (s, 2H), 8.17 (m, 1H), 8.03 (m, 2H), 7.92-7.61 (m, |
| | | | | 4H), 7.47 (m, 2H), 7.09 (d, J = 8.2 Hz, 1H), 6.95 (d, |
| | | | | J = 8.2 Hz, 1H), 1.81 (s, 3H), 1.73 (s, 3H) |
| 49 | J on compound 38 | <10 | m/z | ¹H NMR (400 MHz, DMSO-d6) δ 9.68 (s, 1H), 9.03 |
| | | | 425.3 | (dd, J = 2.4, 0.8 Hz, 1H), 8.56 (dd, J = 4.7, 1.6 Hz, |
| | | | | 1H), 8.27 (dd, J = 2.0, 0.6 Hz, 1H), 8.22 (m, 1H), |
| | | | | 8.12-7.91 (m, 2H), 7.89-7.78 (m, 2H), 7.76 (d, J |
| | | | | = 3.0 Hz, 1H), 7.48 m, 1H), 7.45-7.35 (m, 1H), |
| | | | | 7.09 (dt, J = 8.3, 0.7 Hz, 1H), 6.96 (d, J = 8.3 Hz, |
| | | | | 1H), 1.81 (d, J = 0.7 Hz, 3H), 1.73 (s, 3H). |
| 50 | J on compound 38 | <10 | m/z | ¹H NMR (400 MHz, DMSO-d6) δ 9.63 (s, 1H), 8.18 - |
| | | | 428.3 | 7.93 (m, 3H), 7.83 (dd, J = 8.5, 0.5 Hz, 1H), 7.72 (d, |
| | | | | J = 4.9 Hz, 1H), 7.57 (dd, J = 8.6, 2.1 Hz, 1H), 7.47 |
| | | | | (d, J = 1.9 Hz, 1H), 7.39 (d, J = 3.0 Hz, 1H), 7.09 |
| | | | | (dt, J = 8.3, 0.7 Hz, 1H), 6.95 (d, J = 8.3 Hz, 1H), |
| | | | | 6.50 (d, J = 1.9 Hz, 1H), 3.92 (s, 3H), 1.81 (d, J = |
| | | | | 0.7 Hz, 3H), 1.73 (s, 3H). |
| 51 | J on compound 38 | <10 | m/z | ¹H NMR (400 MHz, DMSO-d6) δ 9.65 (s, 1H), 8.13- |
| | | | 430.3 | 7.87 (m, 3H), 7.76-7.71 (m, 1H), 7.69 (d, J = 8.7 |
| | | | | Hz, 1H), 7.61 (m, 1H), 7.35 (d, J = 3.2 Hz, 1H), 7.08 |
| | | | | (m, 1H), 6.94 (d, J = 8.3 Hz, 1H), 6.43 (m, 1H), 4.23 |
| | | | | (m, 2H), 3.83 (t, J = 5.5 Hz, 2H), 2.56 (m, 2H), 1.79 |
| | | | | (s, 3H), 1.71 (s, 3H). |
| 52 | J on compound 38 | <10 | m/z | ¹H NMR (400 MHz, DMSO-d6) 6 9.61 (s, 1H), 8.10 |
| | | | 552.3 | (dd, J = 2.0, 0.6 Hz, 1H), 7.93 (d, J = 16.6 Hz, 2H), |
| | | | | 7.76 (m, 1H), 7.74 (d, J = 0.5 Hz, 1H), 7.72 (d, J = |
| | | | | 2.0 Hz, 1H), 7.69 (m, 1H), 7.67 (d, J = 1.9 Hz, 1H), |
| | | | | 7.40 - 7.32 (m, 1H), 7.09 (dt, J = 8.3, 0.8 Hz, 1H), |
| | | | | 7.04 - 6.98 (m, 2H), 6.95 (d, J = 8.3 Hz, 1H), 4.42 |
| | | | | (s, 1H), 3.51 (d, J = 12.4 Hz, 2H), 3.17 (m, 4H), 2.55 |
| | | | | (m, 4H), 2.42 (t, J = 6.2 Hz, 2H), 1.85-1.76 (m, |
| | | | | 3H), 1.73 (s, 3H). |
| 53 | J on compound 38 | <10 | m/z | ¹H NMR (400 MHz, DMSO-d6) δ 9.60 (s, 1H), 8.11- |
| | | | 414.3 | 7.84 (m, 2H), 7.81 (m, 1H), 7.75-7.68 (m, 1H), |
| | | | | 7.66 (m, 2H), 7.33 (d, J = 3.3 Hz, 1H), 7.08 (d, J = |
| | | | | 8.3 Hz, 1H), 6.94 (d, J = 8.3 Hz, 1H), 6.43 (m, 1H), |
| | | | | 2.82-2.72 (m, 2H), 2.56-2.48 (m, 2H), 1.97 (p, J |
| | | | | = 7.6 Hz, 2H), 1.79 (s, 3H), 1.71 (s, 3H). |
| 54 | J on compound 38 | <10 | m/z | ¹H NMR (400 MHz, DMSO-d6) δ 12.95 (s, 1H), 9.68 |
| | | | 414.2 | (s, 1H), 8.35 - 8.03 (m, 3H), 7.91 (s, 2H), 7.74 |
| | | | | (d, J = 3.3 Hz, 1H), |
| | | | | 7.73 - 7.66 (m, 2H), 7.35 (d, J = 3.3 Hz, 1H), 7.06 |
| | | | | (d, J = 8.4 Hz, 1H), 6.93 (d, J = 8.3 Hz, 1H), 1.79 (s, |
| | | | | 3H), 1.71 (s, 3H). |
| 55 | A from | <10 | m/z | ¹H NMR (400 MHz, DMSO-d6) δ 9.65 (s, 1H), 8.13 |
| | intermediate S | | 426.1 | (d, J = 23.0 Hz, 2H), 7.97 (d, J = 2.3 Hz, 1H), 7.87 |
| | | | | (d, J = 8.8 Hz, 1H), 7.74 - 7.61 (m, 2H), 7.41 (d, J = |
| | | | | 3.3 Hz, 1H), 7.11 (dt, J = 8.3, 0.8 Hz, 1H), 6.97 (d, |
| | | | | J = 8.3 Hz, 1H), 1.85 - 1.78 (m, 3H), 1.73 (s, 3H). |
| 56 | A from | <10 | m/z | ¹H NMR (400 MHz, DMSO-d6) δ 9.62 (s, 1H), 7.94 |
| | intermediate S | | 426.1 | (m, 3H), 7.84 (d, J = 8.8 Hz, 1H), 7.73-7.55 (m, |
| | | | | 2H), 7.38 (d, J = 3.1 |
| | | | | Hz, 1H), 7.08 (dt, J = 8.2, 0.7 Hz, 1H), 6.94 (d, J = |
| | | | | 8.3 Hz, 1H), 1.83-1.74 (s, 3H), 1.71 (s, 3H). |
| 57 | A from | 1140 | m/z | ¹H NMR (400 MHz, DMSO-d6) 5 9.62 (s, 1H), 8.42- |
| | intermediate S | | 426.1 | 7.91 (m, 3H), 7.84 (d, J = 8.9 Hz, 1H), 7.65 (m, 2H), |
| | | | | 7.38 (s, 1H), 7.08 (dt, J = 8.3, 0.8 Hz, 1H), 6.94 (d, |
| | | | | J = 8.3 Hz, 1H), 1.84-1.74 (s, 3H), 1.71 (s, 3H). |
| 58 | I on compound 55 | 11 | m/z | ¹H NMR (400 MHz, DMSO-d6) δ 9.78 (s, 1H), 7.99 |
| | | | 402.4 | (d, J = 67.1 Hz, 2H), 7.86 (d, J = 8.5 Hz, 1H), 7.73 |
| | | | | (d, J = 1.9 Hz, 1H), 7.67 (s, 1H), 7.52 (dd, J = 8.6, |
| | | | | 1.9 Hz, 1H), 7.39 (s, 1H), 7.08 (d, J = 8.3 Hz, 1H), |
| | | | | 6.95 (d, J = 8.3 Hz, 1H), 4.28 (s, 2H), 1.79 (s, 3H), |
| | | | | 1.71 (s, 3H). |
| 59 | A from | <10 | m/z | ¹H NMR (400 MHz, DMSO-d6) δ 9.68 (s, 1H), 8.12 |
| | intermediate R | | 428.0 | (s, 2H), 7.96 (dd, J = 8.3, 1.3 Hz, 1H), 7.80 (dd, J = |
| | | | | 7.6, 1.3 Hz, 1H), 7.70 (d, J = 3.1 Hz, 1H), 7.49 (dd, |
| | | | | J = 8.3, 7.6 Hz, 1H), 7.44 (d, J = 3.2 Hz, 1H), 7.13 (dt, |
| | | | | J = 8.3, 0.8 Hz, 1H), 6.98 (d, J = 8.3 Hz, 1H), 1.86 |
| | | | | (d, J = 0.7 Hz, 3H), 1.78 (s, 3H). |
| 60 | H on compound 59 | <10 | m/z | ¹H NMR (400 MHz, DMSO-d6) δ 9.64 (s, 1H), 8.44- |
| | | | 373.2 | 8.03 (m, 3H), 7.96 (dd, J = 7.3, 1.3 Hz, 1H), 7.79- |
| | | | | 7.32 (m, 3H), 7.10 (d, J = 8.3 Hz, 1H), 6.95 (d, J = |
| | | | | 8.3 Hz, 1H), 1.82 (s, 3H), 1.74 (s, 3H). |
| 61 | J on compound 59 | <10 | m/z | ¹H NMR (400 MHz, DMSO-d6) δ 9.59 (s, 1H), 8.02 |
| | | | 428.3 | (dd, J = 8.3, 1.5 Hz, 3H), 7.71 (d, J = 3.1 Hz, 1H), |
| | | | | 7.61 (dd, J = 8.3, 7.2 Hz, 1H), 7.44 (dd, J = 7.2, 1.5 |
| | | | | Hz, 1H), 7.41-7.35 (m, 1H), 7.32 (d, J = 1.8 Hz, |
| | | | | 1H), 7.06-6.99 (m, 1H), 6.90 (d, J = 8.3 Hz, 1H), |
| | | | | 6.23 (d, J = 1.8 Hz, 1H), 3.40 (s, 3H), 1.75 (d, J = |
| | | | | 0.7 Hz, 3H), 1.68 (s, 3H). |
| 62 | J on compound 59 | <10 | m/z | ¹H NMR (400 MHz, DMSO-d6) δ 9.57 (s, 1H), 7.98 |
| | | | 414.3 | (s, 2H), 7.79 (dd, J = 8.3, 1.4 Hz, 1H), 7.71-7.59 |
| | | | | (m, 1H), 7.47 (t, J = 7.8 Hz, 1H), 7.38 (dd, J = 7.4, |
| | | | | 1.4 Hz, 1H), 7.35-7.27 (m, 1H), 7.09 (d, J = 8.3 Hz, |
| | | | | 1H), 6.94 (d, J = 8.3 Hz, 1H), 6.47 (t, J = 2.2 Hz, |
| | | | | 1H), 2.71 (m, 2H), 2.36 - 2.23 (m, 2H), 1.82 (s, 3H), |
| | | | | 1.75 (m, 5H). |
| 63 | K on acid | 31 | m/z | ¹H NMR (400 MHz, DMSO-d6) δ 8.22 (s, 1H), 8.11 |
| | intermediate 1 | | Arl+.A: | (br s, 2H), 7.97 (dd, J = 8.5, 2.1 Hz, 1H), 7.78-7.66 |
| | | | | (m, 2H), 7.56 (dd, J = 8.5, 2.0 Hz, 1H), 7.49-7.32 |
| | | | | (m, 1H), 7.12 (d, J = 8.3 Hz, 1H), 6.99 (d, J = 8.3 |
| | | | | Hz, 1H), 3.39 (brs, 4H), 2.31 (brs, 4H), 2.17 (s, |
| | | | | 3H), 1.83 (s, 3H), 1.76 (s, 3H). |
| 64 | K on acid | <10 | m/z | ¹H NMR (400 MHz, DMSO-d6) δ 9.69 (brs, 1H), |
| | intermediate 1 | | 391.4 | 8.41 (d, J = 2.0 Hz, 1H), 8.16 (brs, 2H), 8.04 (dd, |
| | | | | J = 8.7, 2.0 Hz, 1H), 8.01 (brs, 1H), 7.95 (d, J = 8.7 |
| | | | | Hz, 1H), 7.74 (brs, J = 3.1 Hz, 1H), 7.43 (brs, 1H), |
| | | | | 7.35 (brs, 1H), 7.12 (dt, J = 8.3, 0.8 Hz, 1H), 6.99 |
| | | | | (d, J = 8.3 Hz, 1H), 1.84 (s, 3H), 1.76 (s, 3H). |
| 65 | K on acid | 11 | m/z | ¹H NMR (400 MHz, DMSO-d6) δ 9.70 (s, 1H), 8.57 - |
| | intermediate 1 | | 449.3 | 8.43 (m, 1H), 8.37 (d, J = 1.9 Hz, 1H), 8.17 (brs, |
| | | | | 2H), 8.01 (dd, J = 8.7, 2.0 Hz, 1H), 7.95 (d, J = 8.8 |
| | | | | Hz, 1H), 7.73 (brs, 1H), 7.43 (brs, 1H), 7.12 (dt, J = |
| | | | | 8.3, 0.7 Hz, 1H), 6.99 (d, J = 8.3 Hz, 1H), 4.45 (t, J = |
| | | | | 5.2 Hz, 1H), 3.45 (q, J = 6.1 Hz, 2H), 3.39-3.26 (m, |
| | | | | 2H), 1.84 (s, 3H), 1.76 (s, 3H), 1.67 (d, J = 6.8 Hz, |
| | | | | 2H). |
| 66 | K on acid | 13 | m/z | ¹H NMR (400 MHz, DMSO-d6) δ 9.69 (s, 1H), 8.12 |
| | intermediate 1 | | 461.4 | (brs, 2H), 7.98 (d, J = 8.5 Hz, 1H), 7.79 (d, J = 1.8 |
| | | | | Hz, 1H), 7.74 (brd, J = 3.3 Hz, 1H), 7.59 (dd, J = |
| | | | | 8.5, 1.9 Hz, 1H), 7.43 (brs, 1H), 7.13 (dt, J = 8.3, |
| | | | | 0.8 Hz, 1H), 6.98 (d, J = 8.3 Hz, 1H), 3.55 (br m, |
| | | | | 8H), 1.83 (s, 3H), 1.76 (s, 3H). |
| 67 | L on ester | 10 | m/z | ¹H NMR (400 MHz, DMSO-d6) δ 9.66 (s, 1H), 8.03- |
| | intermediate 1 | | 406.5 | 7.90 (m, 2H), 7.88 (dd, J = 8.6, 0.5 Hz, 1H), 7.77 |
| | | | | (dd, J = 2.2, 0.5 Hz, 1H), 7.76-7.66 (m, 2H), 7.34 |
| | | | | (brs, 1H), 7.12 (d, J = 8.3 Hz, 1H), 6.98 (d, J = 8.3 |
| | | | | Hz, 1H), 5.09 (s, 1H), 1.83 (s, 3H), 1.75 (s, 3H), 1.48 |
| | | | | (s, 6H). |
| 68 | L on ester | 13 | m/z | ¹H NMR (400 MHz, DMSO-d6) δ 9.66 (s, 1H), 8.01 |
| | intermediate 1 | | 388.4 | (br s, 2H), 7.89 (dd, J = 7.8, 1.8 Hz, 1H), 7.85 - 7.78 |
| | | | | (m, 2H), 7.73 (d, J = 3.2 Hz, 1H), 7.38 (d, J = 3.2 |
| | | | | Hz, 1H), 7.12 (dt, J = 8.3, 0.8 Hz, 1H), 6.98 (d, J = |
| | | | | 8.3 Hz, 1H), 5.60 (s, 1H), 5.16 (t, J = 1.5 Hz, 1H), |
| | | | | 2.18 (s, 3H), 1.83 (s, 3H), 1.75 (s, 3H). |
| 69 | A, from | 1010 | m/z | ¹H NMR (400 MHz, DMSO-d6) δ 9.74 (s, 1H), 8.29 |
| | intermediate X | | 378.2 | (brs, 2H), 8.03 (dd, J = 7.3, 1.5 Hz, 1H), 7.97 (dd, |
| | | | | J = 8.3, 1.5 Hz, 1H), 7.53 (dd, J = 8.3, 7.3 Hz, 1H), |
| | | | | 7.46 (brs, 2H), 7.31 (dd, J = 8.3, 0.8 Hz, 1H), 6.95 |
| | | | | (dd, J = 8.3, 2.6 Hz, 1H), 6.84 (d, J = 2.6 Hz, 1H), |
| | | | | 1.90 (s, 3H). |
| 70 | A, from | 2300 | m/z | ¹H NMR (400 MHz, DMSO-d6) δ 9.79 (brs, 1H), |
| | intermediate X | | 378.2 | 8.35 (brs, 2H), 8.21 (dd, J = 8.3, 1.5 Hz, 1H), 8.11 |
| | | | | (d, J = 7.3 Hz, 1H), 7.71 (dd, J = 8.3, 7.4 Hz, 1H), |
| | | | | 7.66 (s, 1H), 7.49 (s, 1H), 7.34 (d, J = 8.5 Hz, 1H), |
| | | | | 6.97 (dd, J = 8.3, 2.6 Hz, 1H), 6.89 (d, J = 2.5 Hz, |
| | | | | 1H), 1.94 (s, 3H). |
| 71 | A from | 377 | m/z | ¹H NMR (400 MHz, DMSO-d6) δ 9.72 (s, 1H), 8.14 |
| | intermediate X | | 392.2 | (br s, 2H), 7.95 (dd, J = 8.3, 1.5 Hz, 1H), 7.92-7.84 |
| | | | | (m, 2H), 7.50 (dd, J = 8.3, 7.3 Hz, 1H), 7.41-7.33 |
| | | | | (m, 1H), 7.30 (dd, J = 8.4, 0.8 Hz, 1H), 6.95 (dd, J = |
| | | | | 8.3, 2.6 Hz, 1H), 6.83 (d, J = 2.6 Hz, 1H), 3.92 (s, |
| | | | | 3H), 1.89 (s, 3H). |
| 72 | K on acid | 62 | m/z | ¹H NMR (400 MHz, DMSO-d6) δ 9.76 (brs, 1H), |
| | intermediate 2 | | 377.3 | 8.46 (s, 1H), 8.17 (brs, 2H), 7.92 (dd, J = 7.3, 1.6 |
| | | | | Hz, 1H), 7.87 (dd, J = 8.2, 1.6 Hz, 1H), 7.79 (s, 1H), |
| | | | | 7.49 (brs and dd, J = 8.2, 7.3 Hz, 2H), 7.31 (dd, J = |
| | | | | 8.4, 0.8 Hz, 1H), 6.95 (dd, J = 8.3, 2.6 Hz, 1H), 6.82 |
| | | | | (d, J = 2.5 Hz, 1H), 1.89(S, 3H). |
| 73 | K on acid | 339 | m/z | ¹H NMR (400 MHz, DMSO-d6) δ 9.74 (s, 1H), 8.98 |
| | intermediate 2 | | 405.4 | (t, J = 5.5 Hz, 1H), 8.17 (br s, 2H), 7.93-7.77 (m, |
| | | | | 2H), 7.63-7.38 (m, 3H), 7.31 (dd, J = 8.3, 0.8 Hz, |
| | | | | 1H), 6.95 (dd, J = 8.3, 2.5 Hz, 1H), 6.82 (d, J = 2.6 |
| | | | | Hz, 1H), 3.47-3.36 (m, 2H), 1.88 (s, 3H), 1.21 |
| | | | | (t, J = 7.2 Hz, 3H). |
| 74 | K on acid | 2270 | m/z | ¹H NMR (400 MHz, DMSO-d6) δ 9.74 (brs, 1H), |
| | intermediate 2 | | 405.2 | 8.08 (brs, 2H), 7.81 (dd, J = 7.1,2.6 Hz, 1H), 7.60 |
| | | | | (d, J = 3.3 Hz, 1H), 7.51-7.42 (m, 2H), 7.35 (d, J = |
| | | | | 3.2 Hz, 1H), 7.30 (d, J = 8.5 Hz, 1H), 6.95 (dd, J = |
| | | | | 8.3, 2.6 Hz, 1H), 6.89-6.69 (m, 1H), 3.11 (s, 3H), |
| | | | | 2.73 (d, J = 2.3 Hz, 3H), 1.92 and 1.88 (2 s, 3H). |
| | | | | Splitting coalesced at 60° C. |
| 75 | A from | <10 | m/z | ¹H NMR (400 MHz, DMSO-d6) δ 9.64 (s, 1H), 8.12 |
| | intermediate Z | | 382.2 | (s, 3H), 7.80 (d, J = 3.3 Hz, 1H), 7.76-7.69 (m, |
| | | | | 2H), 7.45 (d, J = 3.3 Hz, 1H), 7.39 (dd, J = 8.3, 7.7 |
| | | | | Hz, 1H), 7.08 (dt, J = 8.3, 0.7 Hz, 1H), 6.95 (d, J = |
| | | | | 8.3 Hz, 1H), 1.79 (d, J = 0.7 Hz, 3H), 1.72 (s, 3H). |
| 76 | A from | <10 | m/z | ¹H NMR (400 MHz, DMSO-d6) δ 9.61 (s, 1H), 8.13 |
| | intermediate Z | | 382.2 | (m, 3H), 7.88 (dd, J = 8.3, 1.4 Hz, 1H), 7.66 (d, J = |
| | | | | 3.2 Hz, 1H), 7.58 (dd, J = 7.6, 1.5 Hz, 1H), 7.53- |
| | | | | 7.47 (m, 1H), 7.45-7.39 (m, 1H), 7.10 (d, J = 8.3 |
| | | | | Hz, 1H), 6.95 (d, J = 8.3 Hz, 1H), 1.81 (s, 3H), 1.73 |
| | | | | (s, 3H). |
| 77 | A from | <10 | m/z | ¹H NMR (400 MHz, DMSO-d6) δ 9.65 (s, 1H), 7.95 |
| | intermediate T | | 362.2 | (s, 2H), 7.74-7.60 (m, 2H), 7.44-7.33 (m, 3H), |
| | | | | 7.08 (dt, J = 8.3, 0.8 Hz, 1H), 6.94 (d, J = 8.3 Hz, |
| | | | | 1H), 5.91 (m, 1H), 4.26 (m, 2H), 3.85 (t, J = 5.3 Hz, |
| | | | | 2H), 2.66 (m, 2H), 1.79 (d, J = 0.7 Hz, 3H), 1.71 (s, |
| | | | | 3H). |
| 78 | A from | <10 | m/z | ¹H NMR (400 MHz, DMSO-d6) δ 9.60 (s, 1H), 8.12 |
| | intermediate T | | 362.2 | (s, 1H), 7.89 (s, 2H), 7.77-7.66 (m, 2H), 7.42 (dd, |
| | | | | J = 8.3, 7.0 Hz, 1H), 7.36-7.26 (m, 2H), 7.09 (dt, J = |
| | | | | 8.3, 0.7 Hz, 1H), 6.93 (d, J = 8.3 Hz, 1H), 2.42 (d, |
| | | | | J = 0.8 Hz, 3H), 1.81 (d, J = 0.7 Hz, 3H), 1.74 (s, 3H). |
| 79 | A from | 30 | m/z | ¹H NMR (400 MHz, DMSO-d6) δ 9.60 (s, 1H), 8.78 |
| | intermediate W | | 429.0 | (d, J = 2.4 Hz, 1H), 8.45 (d, J = 2.3 Hz, 1H), 8.30 (s, |
| | | | | 2H), 7.75-7.57 |
| | | | | (m, 1H), 7.44 (s, 1H), 7.05 (d, J = 8.3 Hz, 1H), 6.92 |
| | | | | (d, J = 8.3 Hz, 1H), 1.77 (s, 3H), 1.69 (s, 3H). |
| 80 | A from | 12 | m/z | ¹H NMR (400 MHz, DMSO-d6) δ 9.62 (s, 1H), 8.83 |
| | intermediate W | | 429.0 | (s, 1H), 8.50 (d, J = 2.2 Hz, 1H), 8.35 (s, 2H), |
| | | | | 7.78-7.34 (m, 2H), 7.08 (d, J = 8.4 Hz, 1H), |
| | | | | 6.94 (d, J = 8.3 Hz, 1H), 1.79 (s, 3H), 1.72 (s, 3H). |
| 81 | A from | 5000 | m/z | ¹H NMR (400 MHz, DMSO-d6) δ 9.62 (s, 1H), 8.83 |
| | intermediate W | | 429.0 | (s, 1H), 8.51 (d, J = 2.2 Hz, 1H), 8.36 (s, 2H), 7.57 |
| | | | | (d, J = 63.4 Hz, 2H), 7.08 (d, J = 8.3 Hz, 1H), 6.94 |
| | | | | (d, J = 8.3 Hz, 1H), 1.79 (s, 3H), 1.72 (s, 3H). |
| 82 | Reduction of | 21 | m/z | ¹H NMR (400 MHz, DMSO-d6) δ 9.65 (s, 1H), 9.09 |
| | compound 79 | | 349.2 | (s, 1H), 8.96-8.84 (m, 2H), 8.82 (dd, J = 8.2, 1.5 |
| | | | | Hz, 1H), 7.78 (dd, J = 8.2, 5.7 Hz, 2H), |
| | | | | 7.48 (s, 1H), 7.07 (d, J = 8.3 Hz, 1H), 6.96 |
| | | | | (d, J = 8.3 Hz, 1H), 1.79 (s, 3H), 1.72 (s, 3H). |
| 83 | A from | 12 | m/z | |
| | intermediate V, | | 363.3 | |
| | then reduction of | | | |
| | Br | | | |
| 84 | A | 11 | m/z | ¹H NMR (400 MHz, DMSO-d6) δ 9.57 (s, 1H), 7.38 |
| | | | 352.4 | (s, 1H), 7.14 (s, 2H), 7.08 (s, 1H), 7.05-6.99 (m, |
| | | | | 1H), 6.89 (d, J = 8.3 Hz, 1H), 2.84 (t, J = 6.0 Hz, |
| | | | | 2H), 2.66 (d, J = 6.1 Hz, 3H), 1.76 (d, J = 6.0 Hz, |
| | | | | 3H), 1.73 (s, 3H), 1.65 (s, 3H). |
| 85 | A from arylamine | 48900 | m/z | |
| | AA14 | | 374.3 | |
| 86 | A | 53 | m/z | ¹H NMR (500 MHz, DMSO-d6) δ 13.33 (s, 1H), 8.03 |
| | | | 358.2 | (brs, 2H), 7.96 (dd, J = 8.4, 1.4 Hz, 1H), 7.81 (brd, |
| | | | | J = 3.3 Hz, 1H), 7.76-7.71 (m, 2H), 7.68 (dd, J = |
| | | | | 8.3, 1.4 Hz, 1H), 7.57 (ddd, J = 8.4, 6.9, 1.5 Hz, 1H), |
| | | | | 7.49 (d, J = 8.5 Hz, 1H), 7.43 (ddd, J = 8.3, 6.9, 1.5 |
| | | | | Hz, 1H), 7.39 (brs, 1H), 2.12 (s, 3H). |
| 87 | A | 10 | m/z | ¹H NMR (400 MHz, DMSO-d6) δ 9.87 (s, 1H), 7.90 |
| | | | 334.1 | (dd, J = 8.3, 1.5 Hz, 2H), 7.78-7.67 (m, 2H), 7.53 |
| | | | | (ddd, J = 8.3, 6.9, 1.5 Hz, 1H), 7.42 (ddd, J = 8.3, |
| | | | | 7.0, 1.5 Hz, 1H), 7.33 (s, 1H), 7.21 (t, J = 7.9 Hz, |
| | | | | 1H), 7.03 (dd, J = 8.2, 1.2 Hz, 1H), 6.85 (dd, J = 7.8, |
| | | | | 1.1 Hz, 1H), 1.76 (s, 3H). |
| 88 | E then G, from | 32 | m/z | ¹H NMR (400 MHz, DMSO-d6) δ ppm 1.67 (s, 3 H), |
| | arylamine AA1 and | | 332.1 | 1.78 (s, 3 H), 6.94 (d, J = 8.3 Hz, 1 H), 7.08 (d, J = |
| | intermediate P | | | 8.3 Hz, 1 H), 7.12 - 7.27 (m, 1 H), 7.59 (ddd, J = 8.0, |
| | | | | 6.9, 1.0 Hz, 1 H), 7.73 (br. s, 1 H), 7.81 (ddd, J = |
| | | | | 8.5, 7.0, 1.3 Hz, 1 H), 8.09 (s, 1 H), 8.16 (d, J = 8.1 |
| | | | | Hz, 1 H), 8.92 (s, 1 H), 9.56 (s, 1 H), 9.65 (d, J = 8.6 |
| | | | | Hz, 1 H). |
| 89 | E then G, from | 31 | m/z | ¹H NMR (400 MHz, DMSO-d6) δ ppm 1.67 (s, 3 H), |
| | arylamine AA1 and | | 336.3 | 1.77 (s, 3 H), 4.15 (s, 3 H), 6.93 (d, J = 8.3Hz, 1 H), |
| | intermediate Q | | | 7.07 (d, J = 8.3 Hz, 1 H), 7.21 (br. s, 1H), 7.55 (br. s, |
| | | | | 1 H), 8.13 (d, J = 1.0 Hz, 1 H), 8.34 (s, 1 H), 8.77 (d, |
| | | | | J = 0.7 Hz, 1 H), 9.56 (s, 1 H). |

In Table 5, the Method column indicates a preparatory method described above used in the preparation of the compounds.

### Example 3. Genetic Validation

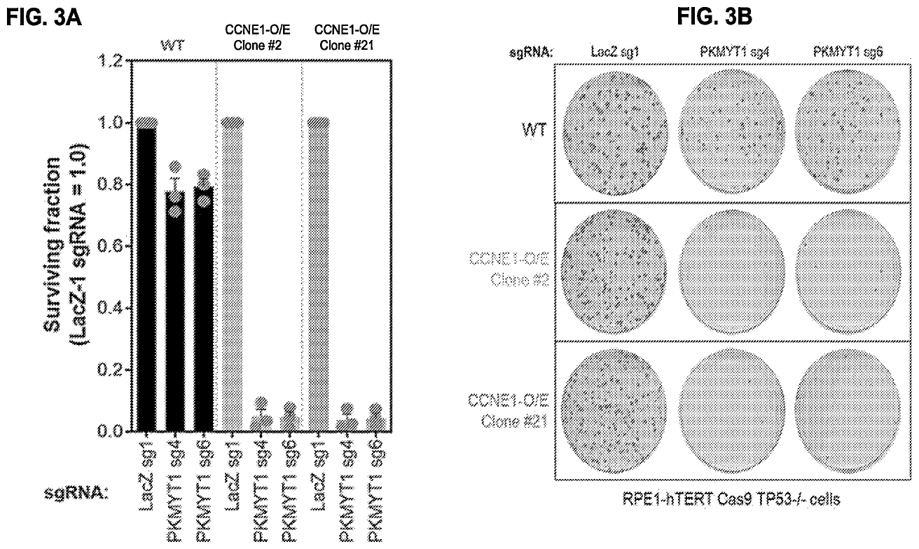

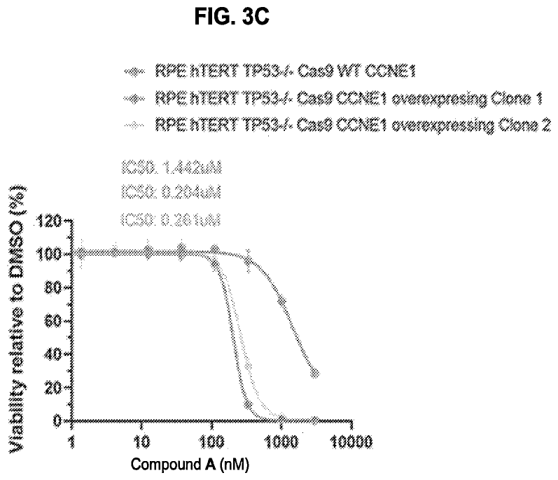

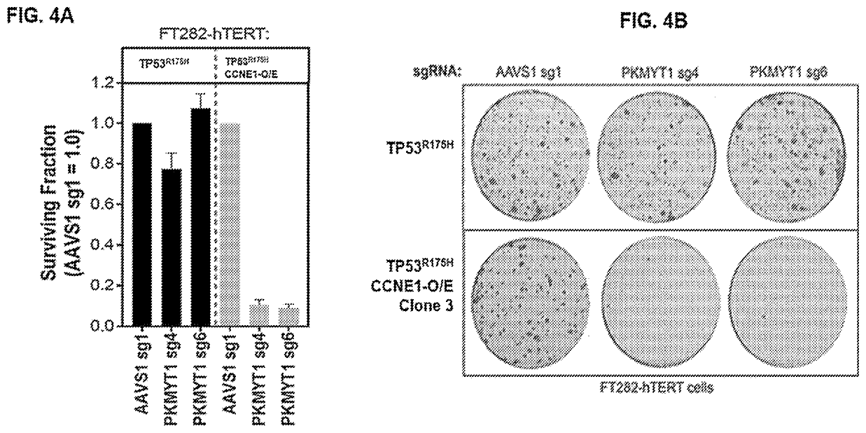

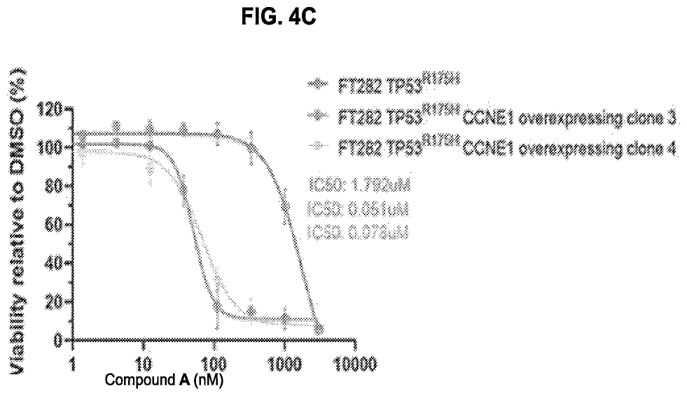

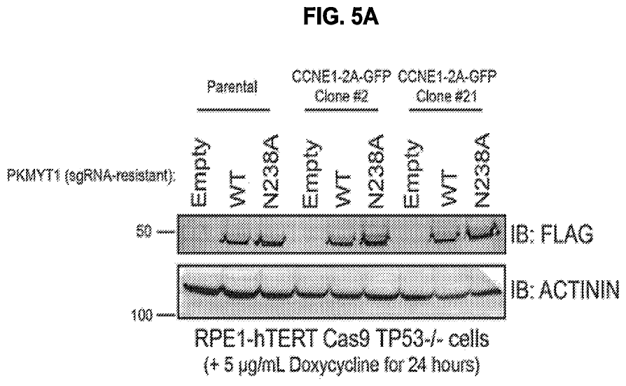

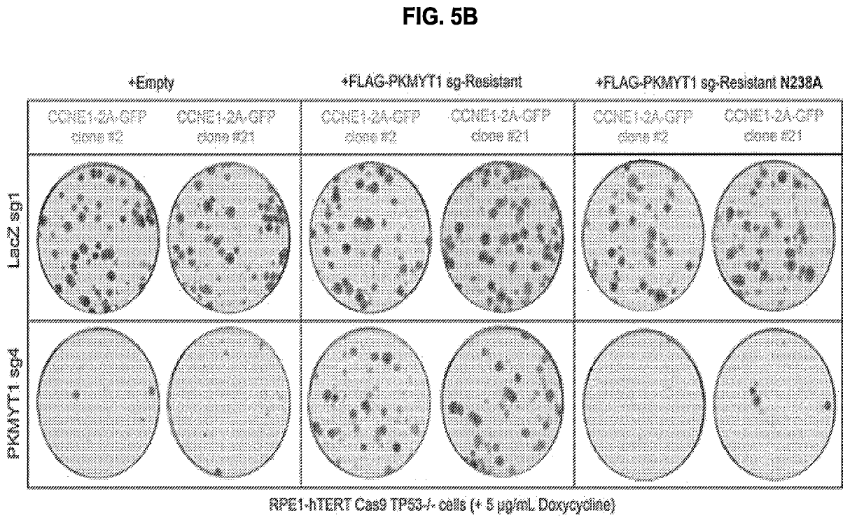

Two sgRNAs for PKMYT1 and one sgRNA for LacZ (control) were transduced into the RPE1-hTERT Cas9 TP53−/− parental (WT) and CCNE1-overexpressing clones. Infected cells were plated at low density to measure their ability to form colonies of <50 cells. After 10 days of growth, the colonies were stained, imaged, and quantified. Using clonogenic survival assays, we observed a profound cellular fitness defect in CCNE1-overexpressing cells compared to parental cells transduced with PKMYT1 sgRNAs (FIGS. 3A and 3B). This experiment was repeated using FT282-hTERT TP53−/− parental (WT) and CCNE1-overexpressing clones and similar results were observed (FIGS. 4A and 4B).

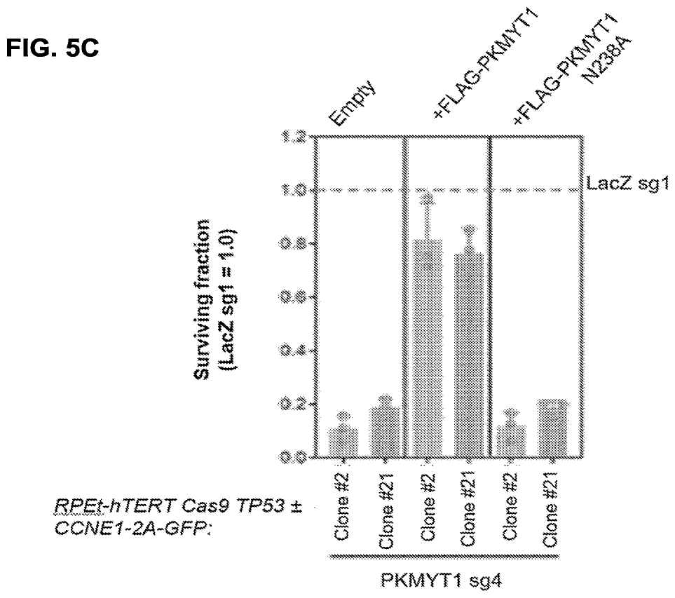

To determine if the kinase activity of PKMYT1 was responsible for maintaining the viability of CCNE1-overexpressing RPE1-hTERT Cas9 TP53−/− cells, the PKMYT1 open reading frame (ORF) was cloned into an inducible mammalian expression vector. sgRNA-resistant silent mutations in the PKMYT1 ORF sequence were then created by PCR mutagenesis. A single point mutation was generated that resulted in an asparagine (N) to alanine (A) amino acid change at residue 238. The N₂₃₈A amino acid change in the kinase domain resulted in a catalytically inactive PKMYT1 mutant. Stable cell lines in the RPE1-hTERT Cas9 TP53−/− parental and CCNE1-overexpressing clones were generated that either expressed the wild type PKMYT1 ORF or the kinase-dead N238A mutant (FIG. 5A). These stable cell lines were transduced with either a LacZ non-targeting sgRNA or PKMYT1 sgRNA #4. The cells were then plated at low density to measure their ability to form colonies of >50 cells. After 10 days of growth, the colonies were stained, imaged, and quantified. Expression of an sgRNA-resistant PKMYT1 ORF but not the catalytic-dead version rescued the fitness defect induced by transduction of sgRNA #4 into both CCNE1-overexpressing clones (FIGS. 5B and 5C). This result demonstrated that targeting the kinase activity of PKMYT1 selectively kills CCNE1-overexpressing cells.

### Example 4. Pharmacological Validation

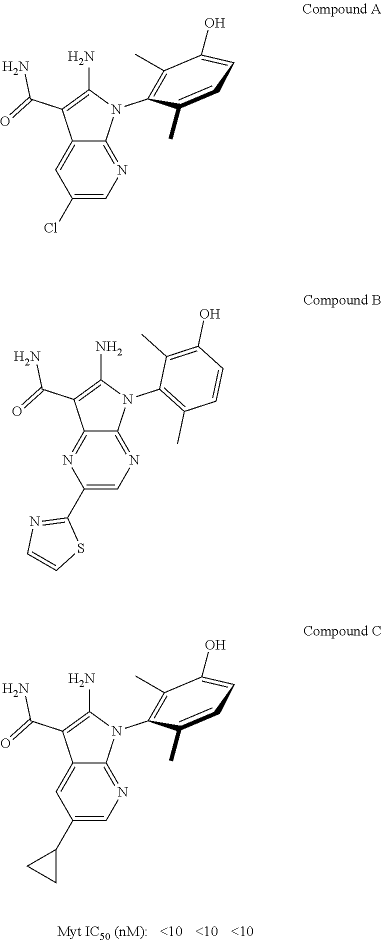

RPE1-hTERT Cas9 TP53−/− parental (WT) and CCNE1-overexpressing clones were treated with compound A in a dose titration and cell viability was determined. The CCNE1-overexpressing cells were found to be more sensitive to compound A than the corresponding WT cells (FIG. 3C). A similar effect was seen in FT282-hTERT TP53^{R175H} WT and CCNE1-overexpressing clones (FIG. 4C). For dose-response proliferation assays using RPE1-hTERT and FT282-hTERT cell lines, cells were seeded in 96-well plates and dosed with serially diluted Myt1 inhibitor. Cells were imaged once per day using the IncuCyte S3 microscope and percent well confluency was calculated overtime. Once cells reached four population doublings the experiment was ended and IC₅₀ curves were plotted for the final time point. Percentage confluency was calculated relative to the cell confluency in the untreated wells.

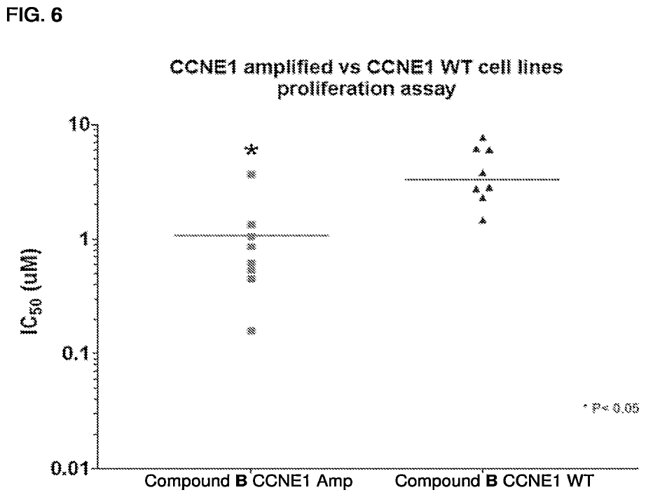

A panel of 16 cancer cell lines with either normal (n=8) or elevated levels of CCNE1 (n=8) was evaluated for their sensitivity to compound B in a cell proliferation assay (FIG. 6). Dose-response curves in these cancer cell line proliferation assays were generated as follows. Cells were seeded in 96-well plates and dosed with serially diluted compound B. After 7 days, Cell Titer Glo (CTG) was used to assess the proliferation status of these cells and the IC₅₀ values were plotted.

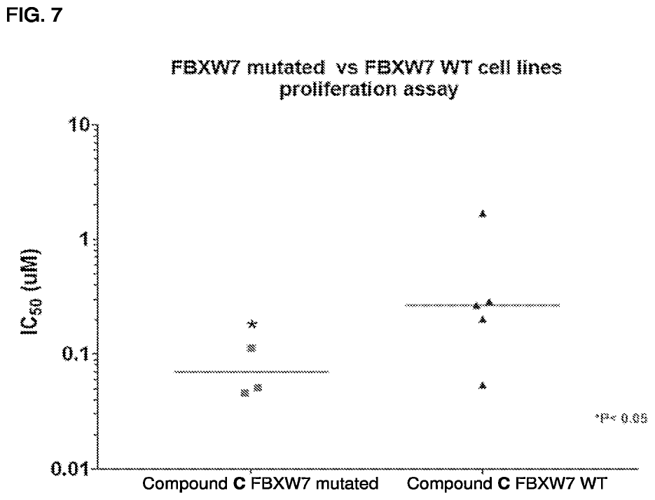

A similar experiment was conducted in a panel of 8 cancer cell lines with either wild-type FBXW7 (n=5) or FBXW7-mutations (n=3) in which these cells were evaluated for their sensitivity to compound C in a cell proliferation assay (FIG. 7). Dose-response curves in these cancer cell line proliferation assays were generated as follows. Cells were seeded in 96-well plates and dosed with serially diluted Myt1 inhibitor. Cells were imaged once per day using the IncuCyte S3 microscope and percent well confluency was calculated over time. Once cells reached four population doublings the experiment was ended and IC₅₀ curves were plotted for the final time point. Percentage confluency was calculated relative to the cell confluency in the untreated wells.

### Other Embodiments

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are in the claims.

1. A compound of formula (IA): 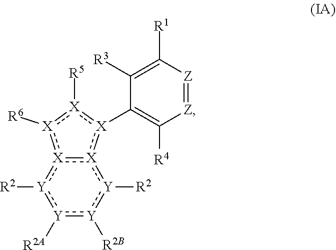
or a pharmaceutically acceptable salt thereof,
wherein
each [symbol] is a single or double bond;
one, two, or three X groups are N, and the remaining X groups are C;
each Y is independently N or C;
each Z is independently N or CH;
R¹ is OH, and R³ is hydrogen, optionally substituted C₁₋₆ alkyl, halogen, or optionally substituted C₃₋₈ cycloalkyl; or R¹ and R³ combine to form —CR⁹═N—NH—;
each R² is independently absent, hydrogen, optionally substituted C₁₋₆ alkyl, optionally substituted C₂-6 alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, cyano, —N(R⁷)₂, —OR⁷, —C(O)N(R⁸)₂, —SO₂N(R⁸)₂, —SO₂R^{7A}, or -Q-R^{7B}; and R^{2A} and R^{2B} together with the atoms to which they are attached, combine to form ring A; or R² and R^{2A}, together with the atoms to which they are attached, combine to form ring A, and R^{2B} is absent or hydrogen;
R⁴ is hydrogen, optionally substituted C₁₋₆ alkyl, halogen, or optionally substituted C₃₋₈ cycloalkyl;
R⁵ is hydrogen, halogen, or —N(R⁷)₂;
R⁶ is —C(O)NH(R⁸), —C(O)R^{7A}, or —SO₂R^{7A};
each R⁷ is independently hydrogen, optionally substituted C₁₋₆ alkyl, optionally substituted C₆₋₁₀ aryl C₁₋₆ alkyl, optionally substituted C₃₋₈ cycloalkyl, optionally substituted C₆₋₁₀ aryl, optionally substituted C₂₋₉ heterocyclyl, optionally substituted C₁₋₉ heteroaryl, optionally substituted C₁₋₉ heteroaryl C₁₋₆ alkyl, or —SO₂R^{7A}; or two R⁷ groups, together with the atom to which both are attached, combine to form an optionally substituted C₂₋₉ heterocyclyl;
each R^{7A} is independently optionally substituted C₁₋₆ alkyl, optionally substituted C₃₋₈ cycloalkyl, or optionally substituted C₆₋₁₀ aryl;
each R^{7B} is independently hydroxyl, optionally substituted C₁₋₆ alkyl, optionally substituted C₆₋₁₀ aryl, optionally substituted C₂₋₉ heterocyclyl, optionally substituted C₁₋₉ heteroaryl, —N(R⁷)₂, —C(O)N(R⁸)₂, —SO₂N(R⁸)₂, —SO₂R^{7A}, or optionally substituted alkoxy;
each R⁸ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or two R⁸, together with the atom to which they are attached, combine to form an optionally substituted C₂₋₉ heterocyclyl;
R⁹ is hydrogen or halogen;
ring A is a 5- or 6-membered carbocyclic ring or a 5- or 6-membered heterocyclic ring, wherein A is optionally substituted with 1, 2, 3, or 4 non-hydrogen R² groups; and
Q is optionally substituted C₁₋₆ alkylene, optionally substituted C₂₋₆ alkenylene, optionally substituted C₂₋₆ alkynylene, optionally substituted C₃₋₈ cycloalkylene, optionally substituted C₃₋₈ cycloalkenylene optionally substituted C₆₋₁₀ arylene, optionally substituted C₂₋₉ heterocyclylene, or optionally substituted C₁₋₉ heteroarylene;
wherein each R² is absent, if attached to Y that is N.

2. A compound of formula (I): 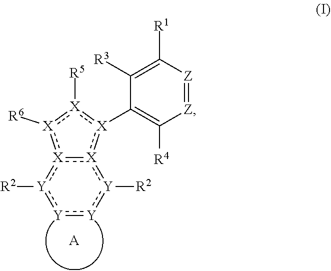
or a pharmaceutically acceptable salt thereof,
wherein
each [symbol] is a single or double bond;
A is a 5- or 6-membered carbocyclic ring or a 5- or 6-membered heterocyclic ring, wherein A is optionally substituted with 1, 2, 3, or 4 non-hydrogen R² groups;
one, two, or three X groups are N, and the remaining X groups are C;
each Y is independently N or C;
each Z is independently N or CH;
R¹ is OH, and R³ is hydrogen, optionally substituted C₁₋₆ alkyl, halogen, or optionally substituted C₃₋₈ cycloalkyl; or R¹ and R³ combine to form —CR⁹═N—NH—;
each R² is independently absent, hydrogen, optionally substituted C₁₋₆ alkyl, optionally substituted C₂₋₆ alkenyl, optionally substituted C₂₋₆ alkynyl, optionally substituted C₃₋₈ cycloalkyl, optionally substituted C₃₋₈ cycloalkenyl, optionally substituted C₂₋₉ heterocyclyl, optionally substituted C₂₋₉ heterocyclyl C₁₋₆ alkyl, optionally substituted C₆₋₁₀ aryl, optionally substituted C₁₋₉ heteroaryl, optionally substituted C₁₋₉ heteroaryl C₁₋₆ alkyl, halogen, cyano, —N(R⁷)₂, —OR⁷, —C(O)N(R⁸)₂, —SO₂N(R⁸)₂, —SO₂R^{7A}, or -Q-R^{7B};
R⁴ is hydrogen, optionally substituted C₁₋₆ alkyl, halogen, or optionally substituted C₃₋₈ cycloalkyl;
R⁵ is hydrogen, halogen, or —N(R⁷)₂;
R⁶ is —C(O)NH(R⁸), —C(O)R^{7A}, or —SO₂R^{7A};
each R⁷ is independently hydrogen, optionally substituted C₁₋₆ alkyl, optionally substituted C₆₋₁₀ aryl C₁₋₆ alkyl, optionally substituted C₃₋₈ cycloalkyl, optionally substituted C₆₋₁₀ aryl, optionally substituted C₂₋₉ heterocyclyl, optionally substituted C₁₋₉ heteroaryl, optionally substituted C₁₋₉ heteroaryl C₁₋₆ alkyl, or —SO₂R^{7A}; or two R⁷ groups, together with the atom to which both are attached, combine to form an optionally substituted C₂₋₉ heterocyclyl;
each R^{7A} is independently optionally substituted C₁₋₆ alkyl, optionally substituted C₃₋₈ cycloalkyl, or optionally substituted C₆₋₁₀ aryl;
each R^{7B} is independently hydroxyl, optionally substituted C₁₋₆ alkyl, optionally substituted C₆₋₁₀ aryl, optionally substituted C₂₋₉ heterocyclyl, optionally substituted C₁₋₉ heteroaryl, —N(R⁷)₂, —C(O)N(R⁸)₂, —SO₂N(R⁸)₂, —SO₂R^{7A}, or optionally substituted alkoxy;
each R⁸ is independently hydrogen, optionally substituted C₁₋₆ alkyl, optionally substituted C₂₋₆ alkoxyalkyl, optionally substituted C₆₋₁₀ aryl C₁₋₆ alkyl, optionally substituted C₆₋₁₀ aryl, optionally substituted C₃₋₈ cycloalkyl, or optionally substituted C₁₋₉ heteroaryl; or two R⁸, together with the atom to which they are attached, combine to form an optionally substituted C₂₋₉ heterocyclyl;
R⁹ is hydrogen or halogen; and
Q is optionally substituted C₁₋₆ alkylene, optionally substituted C₂₋₆ alkenylene, optionally substituted C₂₋₆ alkynylene, optionally substituted C₃₋₈ cycloalkylene, optionally substituted C₃₋₈ cycloalkenylene optionally substituted C₆₋₁₀ arylene, optionally substituted C₂₋₉ heterocyclylene, or optionally substituted C₁₋₉ heteroarylene;
wherein each R² is absent, if attached to Y that is N.

3. The compound of claim 2, or a pharmaceutically acceptable salt thereof, wherein one X group is N, and the remaining X groups are C.

4. (canceled)

5. The compound of claim 1, or a pharmaceutically acceptable salt thereof, wherein all Y groups are C, or one or two Y groups are N and the remaining Y groups are C.

6. (canceled)

7. The compound of claim 1, or a pharmaceutically acceptable salt thereof, wherein the compound is of formula (II):

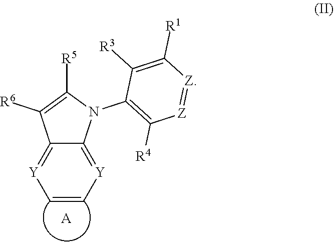

8. The compound of claim 7, or a pharmaceutically acceptable salt thereof, wherein the compound is enriched for the atropisomer of formula (II-i):

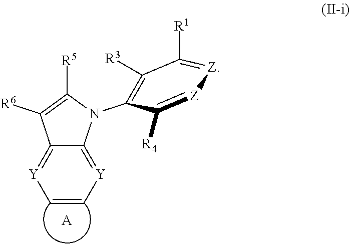

9-34. (canceled)

35. The compound of claim 1, or a pharmaceutically acceptable salt thereof, wherein the compound is of formula (III):

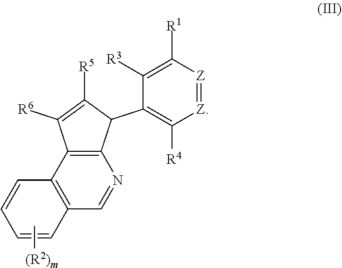

36. The compound of claim 7, or a pharmaceutically acceptable salt thereof, wherein ring A is a 5- or 6-membered heteroaryl ring or 5- or 6-membered carbocyclic ring that is optionally substituted with 1, 2, 3, or 4 non-hydrogen R² groups.

37. (canceled)

38. The compound of claim 36, or a pharmaceutically acceptable salt thereof, wherein Z proximal to R¹ or R⁴ is N.

39-41. (canceled)

42. The compound of any one of claim 36, or a pharmaceutically acceptable salt thereof, wherein R³ is optionally substituted C₁₋₆ alkyl or halogen.

43-44. (canceled)

45. The compound of claim 36, or a pharmaceutically acceptable salt thereof, wherein R¹ and R³ combine to form —CR⁹═N—NH—.

46-47. (canceled)

48. The compound of claim 36, or a pharmaceutically acceptable salt thereof, wherein R⁴ is optionally substituted C₁₋₆ alkyl or halogen.

49. (canceled)

50. The compound of claim 36, or a pharmaceutically acceptable salt thereof, wherein zero, one, two, three, or four R² groups are independently halogen, optionally substituted C₁₋₆ alkyl, methyl, 3-hydroxypropyl, 2-hydroxyprop-2-yl, methoxycarbonyl, hydroxycarbonyl, aminocarbonyl, N,N-dimethylaminocarbonyl, or N-ethylaminocarbonyl, 2-propenyl, optionally substituted heteroaryl, 1-methylpyrazolyl, pyrazolyl, pyridyl, C₃₋₈ cycloalkenyl, cyclopentenyl, bromine, chlorine, fluorine, cyano, -Q-R^{7B}, cyclopropylethynyl, pyrazinylethynyl, 3-(N-morpholinyl)propynyl, 3-hydroxypropynyl, C₆₋₁₀ aryl, phenyl, cyanophenyl, [N-(2-hydroxyethyl)-N′-piperazinyl]phenyl, hydrogen, cyano, N-(2-hydroxyethyl)-N′-piperazinyl, or —C(O)N(R⁸)₂, C₂₋₉ heterocyclyl, or dihydropyranyl, wherein Q is one of optionally substituted C₂₋₆ alkynylene, optionally substituted C₆₋₁₀ arylene, or phenylene, and R^{7B} is hydrogen, cyano, or N-(2-hydroxyethyl)-N′-piperazinyl.

51-88. (canceled)

89. A pharmaceutical composition comprising the compound of claim 36, or a pharmaceutically acceptable salt thereof, and a pharmaceutically acceptable excipient.

90-92. (canceled)

93. A method of inducing cell death in a cancer cell overexpressing CCNE1 or an FBCW7-mutated cancer cell, the method comprising contacting the cell with an effective amount of the compound of claim 1, or a pharmaceutically acceptable salt thereof.

94. The method of claim 93, wherein the cancer cell is a uterine cancer, ovarian cancer, breast cancer, stomach cancer, esophageal cancer, lung cancer, or endometrial cancer cell.

95-98. (canceled)

99. The method of claim 94, wherein the cell is in a subject.
